(19) United States
(12) Patent Application Publication
(10) Pub. No.: US 2002/0169175 A1
(43) Pub. Date: Nov. 14, 2002

(54) Pharmaceutically acceptable salts of heterocyclic compounds

(75) Inventors: Om Reddy Gaddam, Hyderabad (IN); Chandra Sekhar Batchu, Hyderabad (IN); Rajender Kumar Potlapally, Hyderabad (IN); Ramabhadra Sarma Mamillapalli, Hyderabad (IN); Bheema Rao Paraselli, Hyderabad (IN); Naga Venkata Srinivasa Rao Mamidi, Hyderabad (IN)

Correspondence Address: LADAS & PARRY, 26 WEST 61ST STREET, NEW YORK, NY, 10023, US

(73) Assignee: DR. REDDY'S LABORATORIES LTD.

(21) Appl. No.: 10/067,094

(22) Filed: Feb. 4, 2002

Related U.S. Application Data
(60) Provisional application No. 60/266,595, filed on Feb. 5, 2001.

(51) Int. Cl. A61K 31/7008; A61K 31/655; A61K 31/5377; A61K 31/519; A61K 31/517; A61K 31/513; C07H 5/06; C07D 487/02; C07D 239/72

(52) U.S. Cl. 514/262.1; 514/266.3; 514/269; 514/62; 514/252.12; 514/231.2; 536/55.2; 544/262; 544/295; 544/287; 544/314; 514/151

(57) ABSTRACT

The present invention relates to pharmaceutically acceptable salts of the general formula (I), their derivatives, their analogs, their tautomeric forms, their stereoisomers, their polymorphs, their pharmaceutically acceptable solvates and pharmaceutically acceptable compositions containing them.

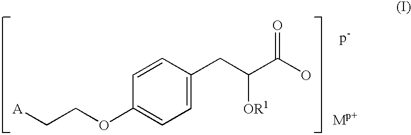

### FIELD OF THE INVENTION

The present invention relates to pharmaceutically acceptable salts of the general formula (I), their derivatives, their analogs, their tautomeric forms, their stereoisomers, their polymorphs, their pharmaceutically acceptable solvates and pharmaceutically acceptable compositions containing them.

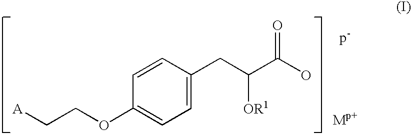

The present invention also relates to a process for the preparation of the above said pharmaceutically acceptable salts, their derivatives, their analogs, their tautomeric forms, their stereoisomers, their polymorphs, their pharmaceutically acceptable solvates, and pharmaceutical compositions containing them.

The compounds of the present invention lower plasma glucose, triglycerides, lower total cholesterol (TC) and increase high density lipoprotein (HDL) and decrease low density lipoprotein (LDL), which have a beneficial effect on coronary heart disease and atherosclerosis.

The compounds of general formula (I) are useful in reducing body weight and for the treatment and/or prophylaxis of diseases such as atherosclerosis, stroke, peripheral vascular diseases and related disorders. These compounds are useful for the treatment of hyperglycemia, hyperlipidemia, hypercholesterolemia, lowering of atherogenic lipoproteins, VLDL (very low density lipoprotein) and LDL. The compounds of the present invention can be used for the treatment of certain renal diseases including glomerulonephritis, glomerulosclerosis, nephrotic syndrome, hypertensive nephrosclerosis and nephropathy. The compounds of general formula (I) are also useful for the treatment and/or prophylaxis of type 2 diabetes, leptin resistance, atherosclerosis, impaired glucose tolerance, disorders related to syndrome X such as hypertension, obesity, insulin resistance, coronary heart disease and other cardiovascular disorders. These compounds may also be useful as aldose reductase inhibitors, for improving cognitive functions in dementia, treating diabetic complications, disorders related to endothelial cell activation, psoriasis, polycystic ovarian syndrome (PCOS), inflammatory bowel diseases, osteoporosis, myotonic dystrophy, pancreatitis, arteriosclerosis, retinopathy, xanthoma, eating disorders, inflammation and for the treatment of cancer. The compounds of the present invention are also useful in the treatment and/or prophylaxis of the above said diseases in combination/concomittant with one or more HMG CoA reductase inhibitors, hypolipidemic/hypolipoproteinemic agents such as fibric acid derivatives, nicotinic acid, cholestyramine, colestipol and probucol.

### BACKGROUND OF INVENTION

Atherosclerosis and other peripheral vascular diseases effect the quality of life of millions of people. Therefore, considerable attention has been directed towards understanding the etiology of hypercholesterolemia and hyperlipidemia and development of effective therapeutic strategies.

Hypercholesterolemia has been defined as plasma cholesterol level that exceeds arbitrarily defined value called “normal” level. Recently, it has been accepted that “ideal” plasma levels of cholesterol are much below the “normal” level of cholesterol in the general population and the risk of coronary artery disease (CAD) increases as cholesterol level rises above the “optimum” (or “ideal”) value. There is clearly a definite cause and effect-relationship between hypercholesterolemia and CAD, particularly for individuals with multiple risk factors. Most of the cholesterol is present in the esterified forms with various lipoproteins such as Low density lipoprotein (LDL), Intermediate density lipoprotein (IDL), High density lipoprotein (HDL) and partially as Very low density lipoprotein (VLDL). Studies clearly indicate that there is an inverse correlationship between CAD and atherosclerosis with serum HDL-cholesterol concentrations, (Stampfer et al., N. Engl. J. Med., 325 (1991), 373-381) and the risk of CAD increases with increasing levels of LDL and VLDL.

In CAD, generally “fatty streaks” in carotid, coronary and cerebral arteries, are found which are primarily free and esterified cholesterol. Miller et al., (Br. Med. J., 282 (1981), 1741-1744) have shown that increase in HDL-particles may decrease the number of sites of stenosis in coronary arteries of human, and high level of HDL-cholesterol may protect against the progression of atherosclerosis. Picardo et al., Arteriosclerosis 6 (1986) 434-441 have shown by in vitro experiment that HDL is capable of removing cholesterol from cells. They suggest that HDL may deplete tissues of excess free cholesterol and transfer it to liver (Macikinnon et al., J. Biol. chem. 261 (1986), 2548-2552). Therefore, agents that increase HDL cholesterol would have therapeutic significance for the treatment of hypercholesterolemia and coronary heart diseases (CHD).

Obesity is a disease highly prevalent in affluent societies and in the developing world and is a major cause of morbidity and mortality. It is a state of excess body fat accumulation. The causes of obesity are unclear. It is believed to be of genetic origin or promoted by an interaction between the genotype and environment. Irrespective of the cause, the result is fat deposition due to imbalance between the energy intake versus energy expenditure. Dieting, exercise and appetite suppression have been a part of obesity treatment. There is a need for efficient therapy to fight this disease since it may lead to coronary heart disease, diabetes, stroke, hyperlipidemia, gout, osteoarthritis, reduced fertility and many other psychological and social problems.

Diabetes and insulin resistance is yet another disease which severely effects the quality of large population in the world. Insulin resistance is the diminished ability of insulin to exert its biological action across a broad range of concentrations. In insulin resistance, the body secretes abnormally high amounts of insulin to compensate for this defect; failing which, the plasma glucose concentration inevitably rises and develops into diabetes. Among the developed countries, diabetes mellitus is a common problem and is associated with a variety of abnormalities including obesity, hypertension, hyperlipidemia (J. Clin. Invest., 75 (1985) 809-817; N. Engl. J. Med 317 (1987) 350-357; J. Clin. Endocrinol. Metab., 66 (1988) 580-583; J. Clin. Invest., 68 (1975) 957-969) and other renal complications (patent publication No. WO 95/21608). It is now increasingly being recognized that insulin resistance and relative hyperinsulinemia have a contributory role in obesity, hypertension, atherosclerosis and type 2 diabetes mellitus. The association of insulin resistance with obesity, hypertension and angina has been described as a syndrome having insulin resistance as the central pathogenic link-Syndrome-X.

Hyperlipidemia is the primary cause for cardiovascular (CVD) and other peripheral vascular diseases. High risk of CVD is related to the higher LDL (Low Density Lipoprotein) and VLDL (Very Low Density Lipoprotein) seen in hyperlipidemia. Patients having glucose intolerance/insulin resistance in addition to hyperlipidemia have higher risk of CVD. Numerous studies in the past have shown that lowering of plasma triglycerides and total cholesterol, in particular LDL and VLDL and increasing HDL cholesterol help in preventing cardiovascular diseases.

Peroxisome proliferator activated receptors (PPAR) are members of the nuclear receptor super family. The gamma (γ) isoform of PPAR (PPARγ) has been implicated in regulating differentiation of adipocytes (Endocrinology, 135 (1994) 798-800) and energy homeostasis (Cell, 83 (1995) 803-812), whereas the alpha (α) isoform of PPAR (PPARα) mediates fatty acid oxidation (Trend. Endocrin. Metab., 4 (1993) 291-296) thereby resulting in reduction of circulating free fatty acid in plasma (Current Biol. 5 (1995) 618-621). PPARα agonists have been found useful for the treatment of obesity (WO 97/36579). It has been recently disclosed that compounds which are agonists for both PPARα. and PPARγ are suggested to be useful for the treatment of syndrome X (WO 97/25042). Similar effect between the insulin sensitizer (PPARγ agonist) and HMG CoA reductase inhibitor has been observed which may be useful for the treatment of atherosclerosis and xanthoma (EP 0 753 298).

It is known that PPARγ plays an important role in adipocyte differentiation (Cell, 87 (1996) 377-389). Ligand activation of PPAR is sufficient to cause complete terminal differentiation (Cell, 79 (1994) 1147-1156) including cell cycle withdrawal. PPARγ is consistently expressed in certain cells and activation of this nuclear receptor with PPARγ agonists would stimulate the terminal differentiation of adipocyte precursors and cause morphological and molecular changes characteristics of a more differentiated, less malignant state (Molecular Cell, (1998), 465-470; Carcinogenesis, (1998), 1949-53; Proc. Natl. Acad. Sci., 94 (1997) 237-241) and inhibition of expression of prostate cancer tissue (Cancer Research 58 (1998) 3344-3352). This would be useful in the treatment of certain types of cancer, which express PPARγ and could lead to a quite nontoxic chemotherapy.

Leptin resistance is a condition wherein the target cells are unable to respond to leptin signal. This may give rise to obesity due to excess food intake and reduced energy expenditure and cause impaired glucose tolerance, type 2 diabetes, cardiovascular diseases and such other interrelated complications. Kallen et al (Proc. Natl. Acad. Sci. (1996) 93, 5793-5796) have reported that insulin sensitizers which perhaps due to the PPAR agonist expression lower plasma leptin concentrations. However, it has been recently disclosed that compounds having insulin sensitizing property also possess leptin sensitization activity. They lower the circulating plasma leptin concentrations by improving the target cell response to leptin (WO 98/02159).

In our WO publication 99/08501 we have disclosed and described the novel compounds of the formula (II),

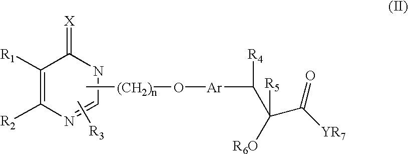

where X represents O or S; the groups R¹, R² and group R³ when attached to the carbon atom, may be same or different and represent hydrogen, halogen, hydroxy, nitro, cyano, formyl or optionally substituted groups selected from alkyl, cycloalkyl, alkoxy, cycloalkoxy, aryl, aryloxy, aralkyl, aralkoxy, heterocyclyl, heteroaryl, heteroaralkyl, heteroaryloxy, heteroaralkoxy, acyl, acyloxy, hydroxyalkyl, amino, acylamino, alkylamino, arylamino, aralkylamino, aminoalkyl, alkoxycarbonyl, aryloxycarbonyl, aralkoxycarbonyl, alkoxyalkyl, aryloxyalkyl, aralkoxyalkyl, alkylthio, thioalkyl, alkoxycarbonylamino, aryloxycarbonylamino, aralkoxycarbonylamino, carboxylic acid or its derivatives, or sulfonic acid or its derivatives; R¹, R² along with the adjacent atoms to which they are attached may also form a substituted or unsubstituted 5-6 membered cyclic structure containing carbon atoms with one or more double bonds, which may optionally contain one or more heteroatoms selected from oxygen, nitrogen and sulfur; R³ when attached to nitrogen atom represents hydrogen, hydroxy, formyl or optionally substituted groups selected from alkyl, cycloalkyl, alkoxy, cycloalkoxy, aryl, aralkyl, heterocyclyl, heteroaryl, heteroaralkyl, acyl, acyloxy, hydroxyalkyl, amino, acylamino, alkylamino, arylamino, aralkylamino, aminoalkyl, aryloxy, aralkoxy, heteroaryloxy, heteroaralkoxy, alkoxycarbonyl, aryloxycarbonyl, aralkoxycarbonyl, alkoxyalkyl, aryloxyalkyl, aralkoxyalkyl, alkylthio, thioalkyl groups, carboxylic acid derivatives, or sulfonic acid derivatives; the linking group represented by —(CH₂)ₙ—O— may be attached either through nitrogen atom or through carbon atom where n is an integer ranging from 1-4; Ar represents an optionally substituted divalent single or fused aromatic or heterocyclic group; R⁴ represents hydrogen atom, hydroxy, alkoxy, halogen, lower alkyl, optionally substituted aralkyl group or forms a bond together with the adjacent group R⁵; R⁵ represents hydrogen, hydroxy, alkoxy, halogen, lower alkyl group, acyl, optionally substituted aralkyl or R⁵ forms a bond together with R⁴; R⁶ may be hydrogen, optionally substituted groups selected from alkyl, cycloalkyl, aryl, aralkyl, alkoxyalkyl, alkoxycarbonyl, aryloxycarbonyl, alkylaminocarbonyl, arylaminocarbonyl, acyl, heterocyclyl, heteroaryl, heteroaralkyl groups, with a provision that R⁶ does not represent hydrogen when R⁷ represents hydrogen or lower alkyl group; R⁷ may be hydrogen or optionally substituted groups selected from alkyl, cycloalkyl, aryl, aralkyl, heterocyclyl, heteroaryl, or heteroaralkyl groups; Y represents oxygen or NR⁸, where R⁸ represents hydrogen, alkyl, aryl, hydroxyalkyl, aralkyl, heterocyclyl, heteroaryl, heteroaralkyl groups; R⁷ and R⁸ together may form a substituted or unsubstituted 5 or 6 membered cyclic structure containing carbon atoms, which may optionally contain one or more heteroatoms selected from oxygen, sulfur or nitrogen. We have also described the processes for preparing the compounds of formula (II).

The pharmaceutically acceptable salts of the general formula (I) have significant formulation and bulk handling advantages in view of the their stability.

### OBJECT OF THE INVENTION

The present invention provides pharmaceutically acceptable salts of β-aryl-α-oxysubstituted alkylcarboxylic acids of the formula (I), their derivatives, their analogs, their tautomeric forms, their stereoisomers, their polymorphs, their pharmaceutically acceptable solvates and pharmaceutical compositions containing them or their mixtures having good stability and solubility, which can be used for the treatment and/or prophylaxis of diseases related to increased levels of lipids, especially to treat hyperlipidemia, and for the treatment of type II diabetes, impaired glucose intolerance, leptin resistance, atherosclerosis, disorders related to Syndrome X such as hypertension, obesity, insulin resistance, coronary artery disease and other cardiovascular disorders, renal diseases including glomerulonephritis, glomerulosclerosis, nephrotic syndrome, hypertensive nephrosclerosis, nephropathy; retinopathy, disorders related to endothelial cell activation, psoriasis, polycystic ovarian syndrome (PCOS), dementia, diabetic complications, eating disorders, osteoporosis, inflammatory bowel diseases, myotonic dystrophy, pancreatitis, retinopathy, arteriosclerosis, xanthoma or cancer with better efficacy, potency and lower toxicity.

The present invention provides pharmaceutically acceptable salts of β-aryl-α-oxysubstituted alkylcarboxylic acids of the formula (I) and their derivatives, their analogs, their tautomeric forms, their stereoisomers, their polymorphs, their pharmaceutically acceptable solvates and pharmaceutical compositions containing them or their mixtures which may have agonist activity against PPARα and/or PPARγ, and optionally inhibit HMG CoA reductase, in addition to agonist activity against PPARα and/or PPARγ.

The present invention provides pharmaceutically acceptable salts of β-aryl-α-oxysubstituted alkylcarboxylic acids of the formula (I) and their derivatives, their analogs, their tautomeric forms, their stereoisomers, their polymorphs and pharmaceutical compositions containing them or their mixtures having enhanced activities, without toxic effect or with reduced toxic effect.

The present invention provides a process for the preparation of pharmaceutically salts of β-aryl-α-oxysubstituted alkylcarboxylic acids and their derivatives of the formula (I) as defined above, their analogs, their tautomeric forms, their stereoisomers and their polymorphs.

The present invention provides pharmaceutical compositions containing compounds of the general formula (I), their analogs, their derivatives, their tautomers, their stereoisomers, their polymorphs or their mixtures in combination with suitable carriers, solvents, diluents and other media normally employed in preparing such compositions.

### DETAILED DESCRIPTION OF THE INVENTION

The present invention relates to pharmaceutically acceptable salts having the general formula (I)

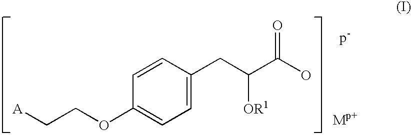

their derivatives, their analogs, their tautomeric forms, their stereoisomers, their polymorphs, and their pharmaceutically acceptable solvates wherein R¹ represents hydrogen, alkyl or aryl group; M represents a counter ion or a moiety which forms a pharmaceutically acceptable salt; p is an integer ranging from 1 to 2; A represents a cyclic structure given below:

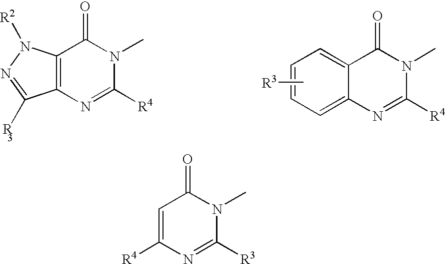

wherein R² and R³ may be same or different and independently represent hydrogen, halogen, hydroxy, nitro, cyano, alkyl or alkoxy group; R⁴ represents hydrogen, halogen, hydroxy, nitro, cyano, azido, formyl or unsubstituted or substituted groups selected from alkyl, cycloalkyl, alkoxy, aryl, heterocyclyl, heteroaryl, amino, monoalkylamino, dialkylamino or alkoxyalkyl groups.

Suitable groups represented by R¹ may be selected from hydrogen, linear or branched (C₁-C₆)alkyl group, such as methyl, ethyl, n-propyl, isopropyl, n-butyl, isobutyl, t-butyl, n-pentyl, isopentyl, hexyl and the like; or aryl group such as phenyl or naphthyl.

Suitable groups represented by R² and R³ may be selected from hydrogen, halogen atom such as fluorine, chlorine, bromine or iodine; hydroxy, nitro, cyano, linear or branched (C₁-C₆)alkyl group, such as methyl, ethyl, n-propyl, isopropyl, n-butyl, isobutyl, t-butyl, n-pentyl, isopentyl, hexyl, heptyl and the like; or linear or branched (C₁-C₆)alkoxy group, such as methoxy, ethoxy, n-propoxy, isopropoxy and the like.

Suitable groups represented by R⁴ may be selected from hydrogen, halogen, hydroxy, nitro, cyano, azido, formyl or unsubstituted or substituted, linear or branched (C₁-C₆)alkyl group, such as methyl, ethyl, n-propyl, isopropyl, n-butyl, isobutyl, t-butyl, n-pentyl, isopentyl, hexyl and the like; unsubstituted or substituted, linear or branched (C₁-C₆)alkoxy group, such as methoxy, ethoxy, n-propoxy, isopropoxy and the like; cyclo(C₃-C₆)alkyl group such as cyclopropyl, cyclobutyl, cyclopentyl, cyclohexyl and the like, the cycloalkyl group may be substituted; aryl group such as phenyl or naphthyl, the aryl group may be substituted; heteroaryl group such as pyridyl, thienyl, furyl, pyrrolyl, oxazolyl, thiazolyl, imidazolyl, oxadiazolyl, tetrazolyl, benzopyranyl, benzofuranyl and the like, the heteroaryl group may be substituted; heterocyclyl groups such as aziridinyl, pyrrolidinyl, morpholinyl, piperidinyl, piperazinyl and the like, the heterocyclyl group may be substituted; amino; monoalkylamino group such as NHCH₃, NHC₂H₅, NHC₃H₇, NHC₆H₁₃, and the like, which may be substituted; dialkylamino group such as N(CH₃)₂, NCH₃(C₂H₅), N(C₂H₅)₂ and the like, which may be substituted; alkoxyalkyl group such as methoxymethyl, ethoxymethyl, methoxyethyl, ethoxyethyl and the like, which may be substituted. The substituents may be selected from halogen atom such as fluorine, chlorine, bromine or iodine; alkyl group such as methyl, ethyl, isopropyl, n-propyl, n-butyl and the like.

Suitable groups represented by M may be selected from sodium, Mg, calcium, potassium, Li, glucamine, N-methyl glucamine, N-octyl glucamine, dicyclohexylamine, t-butyl amine, methyl benzylamine, tris(hydroxymethyl)amino methane (tromethamine), phenyl glycinol, lysine, arginine, metformin, aminoguanidine, aminoguanidine hydrogen carbonate, imidazole, piperazine, dimethyl piperazine, pyrrolidine, benzylamine, phenyl glycine methyl ester, phenylalanine benzyl ester or morpholine.

Particularly useful compounds according to the present invention include:

(±)3-[4-[2-(2-Ethyl-4-oxo-3,4-dihydroquinazolin-3-yl)ethoxy]phenyl]-2-ethoxypropanoic acid phenyl glycinol salt;

(+)3-[4-[2-(2-Ethyl-4-oxo-3,4-dihydroquinazolin-3-yl)ethoxy]phenyl]-2-ethoxypropanoic acid phenyl glycinol salt;

(−)3-[4-[2-(2-Ethyl-4-oxo-3,4-dihydroquinazolin-3-yl)ethoxy]phenyl]-2-ethoxypropanoic acid phenyl glycinol salt;

(±)3-[4-[2-(2-Ethyl-4-oxo-3,4-dihydroquinazolin-3-yl)ethoxy]phenyl]-2-ethoxypropanoic acid methyl benzylamine salt;

(+)3-[4-[2-(2-Ethyl-4-oxo-3,4-dihydroquinazolin-3-yl)ethoxy]phenyl]-2-ethoxypropanoic acid methyl benzylamine salt;

(−)3-[4-[2-(2-Ethyl-4-oxo-3,4-dihydroquinazolin-3-yl)ethoxy]phenyl]-2-ethoxypropanoic acid methyl benzylamine salt;

(±)3-[4-[2-(2-Ethyl-4-oxo-3,4-dihydroquinazolin-3-yl) ethoxy]phenyl]-2-ethoxypropanoic acid dicyclohexylamine salt;

(+)3-[4-[2-(2-Ethyl-4-oxo-3,4-dihydroquinazolin-3-yl)ethoxy]phenyl]-2-ethoxypropanoic acid dicyclohexylamine salt;

(−)3-[4-[2-(2-Ethyl-4-oxo-3,4-dihydroquinazolin-3-yl)ethoxy]phenyl]-2-ethoxypropanoic acid dicyclohexylamine salt;

(±)3-[4-[2-(2-Ethyl-4-oxo-3,4-dihydroquinazolin-3-yl)ethoxy]phenyl]-2-ethoxypropanoic acid lysine salt;

(+)3-[4-[2-(2-Ethyl-4-oxo-3,4-dihydroquinazolin-3-yl)ethoxy]phenyl]-2-ethoxypropanoic acid lysine salt;

(−)3-[4-[2-(2-Ethyl-4-oxo-3,4-dihydroquinazolin-3-yl)ethoxy]phenyl]-2-ethoxypropanoic acid lysine salt;

(±)3-[4-[2-(2-Ethyl-4-oxo-3,4-dihydroquinazolin-3-yl)ethoxy]phenyl]-2-ethoxypropanoic acid tris (hydroxymethyl)amino methane salt;

(+)3-[4-[2-(2-Ethyl-4-oxo-3,4-dihydroquinazolin-3-yl)ethoxy]phenyl]-2-ethoxypropanoic acid tris (hydroxymethyl)amino methane salt;

(−)3-[4-[2-(2-Ethyl-4-oxo-3,4-dihydroquinazolin-3-yl)ethoxy]phenyl]-2-ethoxypropanoic acid tris (hydroxymethyl)amino methane salt;

(±)3-[4-[2-(2-Ethyl-4-oxo-3,4-dihydroquinazolin-3-yl)ethoxy]phenyl]-2-ethoxypropanoic acid N-octyl glucamine salt;

(+)3-[4-[2-(2-Ethyl-4-oxo-3,4-dihydroquinazolin-3-yl)ethoxy]phenyl]-2-ethoxypropanoic acid N-octyl glucamine salt;

(−)3-[4-[2-(2-Ethyl-4-oxo-3,4-dihydroquinazolin-3-yl)ethoxy]phenyl]-2-ethoxypropanoic acid N-octyl glucamine salt;

(±)3-[4-[2-(2-Ethyl-4-oxo-3,4-dihydroquinazolin-3-yl)ethoxy]phenyl]-2-ethoxypropanoic acid N-methyl glucamine salt;

(+)3-[4-[2-(2-Ethyl-4-oxo-3,4-dihydroquinazolin-3-yl)ethoxy]phenyl]-2-ethoxypropanoic acid N-methyl glucamine salt;

(−)3-[4-[2-(2-Ethyl-4-oxo-3,4-dihydroquinazolin-3-yl)ethoxy]phenyl]-2-ethoxypropanoic acid N-methyl glucamine salt;

(±)3-[4-[2-(2-Ethyl-4-oxo-3,4-dihydroquinazolin-3-yl)ethoxy]phenyl]-2-ethoxypropanoic acid amino guanidine hydrogen carbonate salt;

(+)3-[4-[2-(2-Ethyl-4-oxo-3,4-dihydroquinazolin-3-yl)ethoxy]phenyl]-2-ethoxypropanoic acid amino guanidine hydrogen carbonate salt;

(−)3-[4-[2-(2-Ethyl-4-oxo-3,4-dihydroquinazolin-3-yl)ethoxy]phenyl]-2-ethoxypropanoic acid amino guanidine hydrogen carbonate salt;

(±)3-[4-[2-(2-Ethyl-4-oxo-3,4-dihydroquinazolin-3-yl)ethoxy]phenyl]-2-ethoxypropanoic acid lithium salt;

(+)3-[4-[2-(2-Ethyl-4-oxo-3,4-dihydroquinazolin-3-yl)ethoxy]phenyl]-2-ethoxypropanoic acid lithium salt;

(−)3-[4-[2-(2-Ethyl-4-oxo-3,4-dihydroquinazolin-3-yl)ethoxy]phenyl]-2-ethoxypropanoic acid lithium salt;

(±)3-[4-[2-(2-Ethyl-4-oxo-3,4-dihydroquinazolin-3-yl)ethoxy]phenyl]-2-ethoxypropanoic acid arginine salt;

(+)3-[4-[2-(2-Ethyl-4-oxo-3,4-dihydroquinazolin-3-yl)ethoxy]phenyl]-2-ethoxypropanoic acid arginine salt;

(−)3-[4-[2-(2-Ethyl-4-oxo-3,4-dihydroquinazolin-3-yl)ethoxy]phenyl]-2-ethoxypropanoic acid arginine salt;

(±)3-[4-[2-(2-Ethyl-4-oxo-3,4-dihydroquinazolin-3-yl)ethoxy]phenyl]-2-ethoxypropanoic acid metformin salt;

(+)3-[4-[2-(2-Ethyl-4-oxo-3,4-dihydroquinazolin-3-yl)ethoxy]phenyl]-2-ethoxypropanoic acid metformin salt;

(−)3-[4-[2-(2-Ethyl-4-oxo-3,4-dihydroquinazolin-3-yl)ethoxy]phenyl]-2-ethoxypropanoic acid metformin salt;

(±)3-[4-[2-(2-Ethyl-4-oxo-3,4-dihydroquinazolin-3-yl)ethoxy]phenyl]-2-ethoxypropanoic acid imidazole salt;

(+)3-[4-[2-(2-Ethyl-4-oxo-3,4-dihydroquinazolin-3-yl)ethoxy]phenyl]-2-ethoxypropanoic acid imidazole salt;

(−)3-[4-[2-(2-Ethyl-4-oxo-3,4-dihydroquinazolin-3-yl)ethoxy]phenyl]-2-ethoxypropanoic acid imidazole salt;

(±)3-[4-[2-(2-Ethyl-4-oxo-3,4-dihydroquinazolin-3-yl)ethoxy]phenyl]-2-ethoxypropanoic acid magnesium salt;

(+)3-[4-[2-(2-Ethyl-4-oxo-3,4-dihydroquinazolin-3-yl)ethoxy]phenyl]-2-ethoxypropanoic acid magnesium salt;

(−)3-[4-[2-(2-Ethyl-4-oxo-3,4-dihydroquinazolin-3-yl)ethoxy]phenyl]-2-ethoxypropanoic acid magnesium salt;

(±)3-[4-[2-(2-Morpholinyl-4-oxo-3,4-dihydroquinazolin-3-yl)ethoxy]phenyl]-2-ethoxypropanoic acid magnesium salt;

(+)3-[4-[2-(2-Morpholinyl-4-oxo-3,4-dihydroquinazolin-3-yl)ethoxy]phenyl]-2-ethoxypropanoic acid magnesium salt;

(−)3-[4-[2-(2-Morpholinyl-4-oxo-3,4-dihydroquinazolin-3-yl)ethoxy]phenyl]-2-ethoxypropanoic acid magnesium salt;

(±)3-[4-[2-(2-Piperidinyl-4-oxo-3,4-dihydroquinazolin-3-yl)ethoxy]phenyl]-2-ethoxypropanoic acid magnesium salt;

(+)3-[4-[2-(2-Piperidinyl-4-oxo-3,4-dihydroquinazolin-3-yl)ethoxy]phenyl]-2-ethoxypropanoic acid magnesium salt;

(−)3-[4-[2-(2-Piperidinyl-4-oxo-3,4-dihydroquinazolin-3-yl)ethoxy]phenyl]-2-ethoxypropanoic acid magnesium salt;

(±)3-[4-[2-(2-Azido-4-oxo-3,4-dihydroquinazolin-3-yl)ethoxy]phenyl]-2-ethoxypropanoic acid magnesium salt;

(+)3-[4-[2-(2-Azido-4-oxo-3,4-dihydroquinazolin-3-yl)ethoxy]phenyl]-2-ethoxypropanoic acid magnesium salt;

(−)3-[4-[2-(2-Azido-4-oxo-3,4-dihydroquinazolin-3-yl)ethoxy]phenyl]-2-ethoxypropanoic acid magnesium salt;

(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid magnesium salt;

(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid magnesium salt;

(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid magnesium salt;

(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid magnesium salt;

(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid magnesium salt;

(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid magnesium salt;

(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid potassium salt;

(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid potassium salt;

(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid potassium salt;

(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid potassium salt;

(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid potassium salt;

(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid potassium salt

(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid calcium salt;

(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid calcium salt;

(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid calcium salt;

(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid calcium salt;

(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid calcium salt;

(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid calcium salt;

(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid lithium salt;

(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid lithium salt;

(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid lithium salt;

(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid lithium salt;

(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid lithium salt;

(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid lithium salt;

(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid sodium salt;

(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid sodium salt;

(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid sodium salt;

(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid sodium salt;

(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid sodium salt;

(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid sodium salt;

(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid arginine salt;

(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid arginine salt;

(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid arginine salt;

(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid arginine salt;

(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid arginine salt;

(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid arginine salt;

(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid methyl benzylamine salt;

(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid methyl benzylamine salt;

(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid methyl benzylamine salt;

(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid methyl benzylamine salt;

(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid methyl benzylamine salt;

(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid methyl benzylamine salt;

(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid S-(+)-phenylglycinol salt;

(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid S-(+)-phenylglycinol salt;

(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid S-(+)-phenylglycinol salt;

(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid S-(+)-phenylglycinol salt;

(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid S-(+)-phenylglycinol salt;

(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid S-(+)-phenylglycinol salt;

(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid aminoguanidine salt;

(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid aminoguanidine salt;

(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid aminoguanidine salt;

(±)3-[4-[2-(1,5-Dimethyl-7-oxo-3-propyl-6,7-dihydro-1H-pyrazolo[4,3-d]pyrimidin-6-yl ethoxy]phenyl]-2-ethoxypropionic acid aminoguanidine salt;

(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid aminoguanidine salt;

(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid aminoguanidine salt;

(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid tromethamine salt;

(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid tromethamine salt;

(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid tromethamine salt;

(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid tromethamine salt;

(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid tromethamine salt;

(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid tromethamine salt;

(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid dicyclohexylamine salt;

(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid dicyclohexylamine salt;

(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid dicyclohexylamine salt;

(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid dicyclohexylamine salt;

(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid dicyclohexylamine salt;

(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid dicyclohexylamine salt;

(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid N-octylglucamine salt;

(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid N-octylglucamine salt;

(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid N-octylglucamine salt;

(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid N-octylglucamine salt;

(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid N-octylglucamine salt;

(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid N-octylglucamine salt;

(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid N-methylglucamine salt;

(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid N-methylglucamine salt;

(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid N-methylglucamine salt;

(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid N-methylglucamine salt;

(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid N-methylglucamine salt;

(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid N-methylglucamine salt;

(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid metformin salt;

(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid metformin salt;

(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid metformin salt;

(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid metformin salt;

(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid metformin salt;

(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid metformin salt;

(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid lysine salt;

(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid lysine salt;

(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid lysine salt;

(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid lysine salt;

(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid lysine salt;

(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid lysine salt;

(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid t-butylamine salt;

(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid t-butylamine salt;

(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid t-butylamine salt;

(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid t-butylamine salt;

(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid t-butylamine salt;

(−)-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid t-butylamine salt;

(±)-Ethoxy-3-[4-[2-[2-ethyl-6-oxo-4-phenyl-1,6-dihydropyrimidin-1-yl]ethoxy]phenyl]propanoic acid potassium salt;

(+)-Ethoxy-3-[4-[2-[2-ethyl-6-oxo-4-phenyl-1,6-dihydropyrimidin-1-yl]ethoxy]phenyl]propanoic acid potassium salt;

(−)-Ethoxy-3-[4-[2-[2-ethyl-6-oxo-4-phenyl-1,6-dihydropyrimidin-1-yl]ethoxy]phenyl]propanoic acid potassium salt;

(±)-Ethoxy-3-[4-[2-[2-ethyl-6-oxo-4-phenyl-1,6-dihydropyrimidin-1-yl]ethoxy]phenyl]propanoic acid magnesium salt;

(+)-Ethoxy-3-[4-[2-[2-ethyl-6-oxo-4-phenyl-1,6-dihydropyrimidin-1-yl]ethoxy]phenyl]propanoic acid magnesium salt;

(−)-Ethoxy-3-[4-[2-[2-ethyl-6-oxo-4-phenyl-1,6-dihydropyrimidin-1-yl]ethoxy]phenyl]propanoic acid magnesium salt;

(±)-Ethoxy-3-[4-[2-[2-ethyl-6-oxo-4-phenyl-1,6-dihydropyrimidin-1-yl]ethoxy]phenyl]propanoic acid phenyl glycinol salt;

(+)-Ethoxy-3-[4-[2-[2-ethyl-6-oxo-4-phenyl-1,6-dihydropyrimidin-1-yl]ethoxy]phenyl]propanoic acid phenyl glycinol salt;

(−)-Ethoxy-3-[4-[2-[2-ethyl-6-oxo-4-phenyl-1,6-dihydropyrimidin-1-yl]ethoxy]phenyl]propanoic acid phenyl glycinol salt;

(±)-Ethoxy-3-[4-[2-[2-ethyl-6-oxo-4-phenyl-1,6-dihydropyrimidin-1-yl]ethoxy]phenyl]propanoic acid sodium salt;

(+)-Ethoxy-3-[4-[2-[2-ethyl-6-oxo-4-phenyl-1,6-dihydropyrimidin-1-yl]ethoxy]phenyl]propanoic acid sodium salt;

(−)-Ethoxy-3-[4-[2-[2-ethyl-6-oxo-4-phenyl-1,6-dihydropyrimidin-1-yl]ethoxy]phenyl]propanoic acid sodium salt;

(±)-Ethoxy-3-[4-[2-[2-ethyl-6-oxo-4-phenyl-1,6-dihydropyrimidin-1-yl]ethoxy]phenyl]propanoic acid L-lysine salt;

(+)-Ethoxy-3-[4-[2-[2-ethyl-6-oxo-4-phenyl-1,6-dihydropyrimidin-1-yl]ethoxy]phenyl]propanoic acid L-lysine salt;

(−)-Ethoxy-3-[4-[2-[2-ethyl-6-oxo-4-phenyl-1,6-dihydropyrimidin-1-yl]ethoxy]phenyl]propanoic acid L-lysine salt;

(±)-Ethoxy-3-[4-[2-[2-ethyl-6-oxo-4-phenyl-1,6-dihydropyrimidin-1-yl]ethoxy]phenyl]propanoic acid t-butylamine salt;

(+)-Ethoxy-3-[4-[2-[2-ethyl-6-oxo-4-phenyl-1,6-dihydropyrimidin-1-yl]ethoxy]phenyl]propanoic acid t-butylamine salt;

(−)-Ethoxy-3-[4-[2-[2-ethyl-6-oxo-4-phenyl-1,6-dihydropyrimidin-1-yl]ethoxy]phenyl]propanoic acid t-butylamine salt;

(±)-Ethoxy-3-[4-[2-[2-ethyl-6-oxo-4-phenyl-1,6-dihydropyrimidin-1-yl]ethoxy]phenyl]propanoic acid N-methyl glucamine salt;

(+)-Ethoxy-3-[4-[2-[2-ethyl-6-oxo-4-phenyl-1,6-dihydropyrimidin-1-yl]ethoxy]phenyl]propanoic acid N-methyl glucamine salt;

(−)-Ethoxy-3-[4-[2-[2-ethyl-6-oxo-4-phenyl-1,6-dihydropyrimidin-1-yl]ethoxy]phenyl]propanoic acid N-methyl glucamine salt;

(±)-Ethoxy-3-[4-[2-[2-ethyl-6-oxo-4-phenyl-1,6-dihydropyrimidin-1-yl]ethoxy]phenyl]propanoic acid N-octyl glucamine salt;

(+)-Ethoxy-3-[4-[2-[2-ethyl-6-oxo-4-phenyl-1,6-dihydropyrimidin-1-yl]ethoxy]phenyl]propanoic acid N-octyl glucamine salt;

(−)-Ethoxy-3-[4-[2-[2-ethyl-6-oxo-4-phenyl-1,6-dihydropyrimidin-1-yl]ethoxy]phenyl]propanoic acid N-octyl glucamine salt;

(±)-Ethoxy-3-[4-[2-[2-ethyl-6-oxo-4-phenyl-1,6-dihydropyrimidin-1-yl]ethoxy]phenyl]propanoic acid tris (hydroxymethyl) amino methane salt;

(+)-Ethoxy-3-[4-[2-[2-ethyl-6-oxo-4-phenyl-1,6-dihydropyrimidin-1-yl]ethoxy]phenyl]propanoic acid tris (hydroxymethyl) amino methane salt;

(−)2-Ethoxy-3-[4-[2-[2-ethyl-6-oxo-4-phenyl-1,6-dihydropyrimidin-1-yl]ethoxy]phenyl]propanoic acid tris (hydroxymethyl)amino methane salt;

(±)-Ethoxy-3-[4-[2-[2-ethyl-6-oxo-4-phenyl-1,6-dihydropyrimidin-1-yl]ethoxy]phenyl]propanoic acid lithium salt;

(+)-Ethoxy-3-[4-[2-[2-ethyl-6-oxo-4-phenyl-1,6-dihydropyrimidin-1-yl]ethoxy]phenyl]propanoic acid lithium salt;

(−)-Ethoxy-3-[4-[2-[2-ethyl-6-oxo-4-phenyl-1,6-dihydropyrimidin-1-yl]ethoxy]phenyl]propanoic acid lithium salt;

(±)-Ethoxy-3-[4-[2-[2-ethyl-6-oxo-4-phenyl-1,6-dihydropyrimidin-1-yl]ethoxy]phenyl]propanoic acid calcium salt;

(+)-Ethoxy-3-[4-[2-[2-ethyl-6-oxo-4-phenyl-1,6-dihydropyrimidin-1-yl]ethoxy]phenyl]propanoic acid calcium salt;

(−)-Ethoxy-3-[4-[2-[2-ethyl-6-oxo-4-phenyl-1,6-dihydropyrimidin-1-yl]ethoxy]phenyl]propanoic acid calcium salt;

(±)-Ethoxy-3-[4-[2-[2-ethyl-6-oxo-4-phenyl-1,6-dihydropyrimidin-1-yl]ethoxy]phenyl]propanoic acid L-arginine salt;

(+)-Ethoxy-3-[4-[2-[2-ethyl-6-oxo-4-phenyl-1,6-dihydropyrimidin-1-yl]ethoxy]phenyl]propanoic acid L-arginine salt;

(−)-Ethoxy-3-[4-[2-[2-ethyl-6-oxo-4-phenyl-1,6-dihydropyrimidin-1-yl]ethoxy]phenyl]propanoic acid L-arginine salt;

(±)2-Ethoxy-3-[4-[2-[2-ethyl-6-oxo-4-phenyl-1,6-dihydropyrimidin-1-yl]ethoxy]phenyl]propanoic acid metformin salt;

(+)2-Ethoxy-3-[4-[2-[2-ethyl-6-oxo-4-phenyl-1,6-dihydropyrimidin-1-yl]ethoxy]phenyl]propanoic acid metformin salt;

(−)2-Ethoxy-3-[4-[2-[2-ethyl-6-oxo-4-phenyl-1,6-dihydropyrimidin-1-yl]ethoxy]phenyl]propanoic acid metformin salt;

(±)2-Ethoxy-3-[4-[2-[2-ethyl-6-oxo-4-phenyl-1,6-dihydropyrimidin-1-yl]ethoxy]phenyl]propanoic acid dicyclohexylamine salt;

(+)2-Ethoxy-3-[4-[2-[2-ethyl-6-oxo-4-phenyl-1,6-dihydropyrimidin-1-yl]ethoxy]phenyl]propanoic acid dicyclohexylamine salt;

(−)2-Ethoxy-3-[4-[2-[2-ethyl-6-oxo-4-phenyl-1,6-dihydropyrimidin-1-yl]ethoxy]phenyl]propanoic acid dicyclohexylamine salt;

(±)2-Ethoxy-3-[4-[2-[2-ethyl-6-oxo-4-phenyl-1,6-dihydropyrimidin-1-yl]ethoxy]phenyl]propanoic acid aminoguanidine salt;

(+)2-Ethoxy-3-[4-[2-[2-ethyl-6-oxo-4-phenyl-1,6-dihydropyrimidin-1-yl]ethoxy]phenyl]propanoic acid aminoguanidine salt;

(−)2-Ethoxy-3-[4-[2-[2-ethyl-6-oxo-4-phenyl-1,6-dihydropyrimidin-1-yl]ethoxy]phenyl]propanoic acid aminoguanidine salt;

(±)2-Ethoxy-3-[4-[2-[2-ethyl-6-oxo-4-phenyl-1,6-dihydropyrimidin-1-yl]ethoxy]phenyl]propanoic acid methyl benzylamine salt;

(+)2-Ethoxy-3-[4-[2-[2-ethyl-6-oxo-4-phenyl-1,6-dihydropyrimidin-1-yl]ethoxy]phenyl]propanoic acid methyl benzylamine salt;

(−)2-Ethoxy-3-[4-[2-[2-ethyl-6-oxo-4-phenyl-1,6-dihydropyrimidin-1-yl]ethoxy]phenyl]propanoic acid methyl benzylamine salt;

(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid aminoguanidine hydrogen carbonate salt;

(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid aminoguanidine hydrogen carbonate salt;

(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid aminoguanidine hydrogen carbonate salt;

(±)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid aminoguanidine hydrogen carbonate salt;

(+)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid aminoguanidine hydrogen carbonate salt; and

(−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid aminoguanidine hydrogen carbonate salt.

According to another feature of the present invention, there is provided a process for the preparation of pharmaceutically acceptable salts of the formula (I) which comprises, reacting compound of the formula (III)

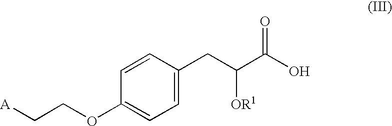

where all symbols are as defined earlier with a stoichiometric amount of an appropriate base in the presence of a solvent at a temperature in the range of −10° C. to the boiling point of the solvent employed for a period in the range of 10 minutes to 30 hours.

The compound of the formula (III) used may be either optically pure form or a racemic form. The base employed in the reaction may be selected from sodium hydroxide, sodium methoxide, potassium hydroxide, calcium hydroxide, lithium hydroxide, magnesium hydroxide, glucamine, N-methylglucamine, N-octylglucamine, dicyclohexylamine, t-butylamine, methyl benzylamine, tris(hydroxymethyl)aminomethane, phenyl glycinol, lysine, arginine, metformin, aminoguanidine, aminoguanidine hydrogen carbonate, imidazole, piperazine, dimethyl piperazine, pyrrolidine, benzylamine, phenyl glycine methyl ester, phenylalanine benzyl ester or morpholine. The solvent employed may be selected from alcohols such as ethanol, methanol, isopropanol, butanol and the like; ketones such as acetone, diethyl ketone, methyl ethyl ketone or their mixtures; ethers such as diethyl ether, ether, tetrahydrofuran, dioxane, dibutyl ether and the like or DMF, DMSO, xylene, toluene, ethyl acetate and the like or mixture thereof.

The pharmaceutically acceptable salts of the general formula (I) have significant formulation and bulk handling advantages in view of the their physicochemical properties and their stability.

Various polymorphs of a compound of general formula (I) forming part of this invention may be prepared by crystallization of compound of formula (I) under different conditions. For example, using different solvents commonly used or their mixtures for recrystallization; crystallizations at different temperatures; various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffraction or such other techniques.

The stereoisomers of the compounds forming part of this invention may be prepared by using compound of formula (I) in its single enantiomeric form in the process by resolving the mixture of stereoisomers by conventional methods. Some of the preferred methods include use of microbial resolution, resolving the diastereomeric salts formed with optically pure bases such as brucine, cinchona alkaloids and their derivatives, optically pure 2-alkyl phenethyl amine, phenyl glycinol and the like. The diastereomeric salts may be obtained in pure form by fractional crystallization. Commonly used methods are compiled by Jaques et al in “Enantiomers, Racemates and Resolution” (Wiley Interscience, 1981).

Pharmaceutically acceptable solvates of the compounds of formula (I) forming part of this invention may be prepared by conventional methods such as dissolving the compounds of formula (I) in solvents such as water, methanol, ethanol and the like, preferably water and recrystallizing by using different crystallization techniques.

The present invention provides a pharmaceutical composition, containing the compounds of the general formula (I) as defined above, their derivatives, their analogs, their tautomeric forms, their stereoisomers, their polymorphs, and/or their pharmaceutically acceptable solvates in combination with the usual pharmaceutically employed carriers, diluents and the like, useful for the treatment and/or prophylaxis of diseases such as hypertension, coronary heart disease, atherosclerosis, stroke, peripheral vascular diseases and related disorders. These compounds are useful for the treatment of familial hypercholesterolemia, hypertriglyceridemia, lowering of atherogenic lipoproteins, VLDL and LDL. The compounds of the present invention can be used for the treatment of certain renal diseases including glomerulonephritis, glomerulosclerosis, nephrotic syndrome, hypertensive nephrosclerosis, and nephropathy. The compounds of general formula (I) are also useful for the treatment/prophylaxis of insulin resistance (type II diabetes), leptin resistance, impaired glucose tolerance, dyslipidemia, disorders related to syndrome X such as hypertension, obesity, insulin resistance, coronary heart disease, and other cardiovascular disorders. These compounds may also be useful as aldose reductase inhibitors, for improving cognitive functions in dementia, as inflammatory agents, treating diabetic complications, disorders related to endothelial cell activation, psoriasis, polycystic ovarian syndrome (PCOS), inflammatory bowel diseases, osteoporosis, myotonic dystrophy, pancreatitis, retinopathy, arteriosclerosis, xanthoma and for the treatment of cancer. The compounds of the present invention are useful in the treatment and/or prophylaxis of the above said diseases in combination/concomittant with one or more HMG CoA reductase inhibitors, hypolipidemic/hypolipoproteinemic agents such as fibric acid derivatives, nicotinic acid, cholestyramine, colestipol, probucol or their combination. The compounds of the present invention in combination with HMG CoA reductase inhibitors, hypolipidemic/hypolipoproteinemic agents can be administered together or within such a period to act synergistically. The HMG CoA reductase inhibitors may be selected from those used for the treatment or prevention of hyperlipidemia such as lovastatin, provastatin, simvastatin, fluvastatin, atorvastatin, cerivastatin and their analogs thereof. Suitable fibric acid derivative may be gemfibrozil, clofibrate, fenofibrate, ciprofibrate, benzafibrate and their analogs thereof.

The present invention also provides a pharmaceutical composition, containing the compounds of the general formula (I) as defined above, their derivatives, their analogs, their tautomeric forms, their stereoisomers, their polymorphs, their pharmaceutically acceptable solvates and one or more HMG CoA reductase inhibitors, hypolipidemic/hypolipoproteinemic agents such as fibric acid derivatives, nicotinic acid, cholestyramine, colestipol, probucol in combination with the usual pharmaceutically employed carriers, diluents and the like.

The pharmaceutical composition may be in the forms normally employed, such as tablets, capsules, powders, syrups, solutions, suspensions and the like, may contain flavorants, sweeteners etc. in suitable solid or liquid carriers or diluents, or in suitable sterile media to form injectable solutions or suspensions. Such compositions typically contain from 1 to 20%, preferably 1 to 10% by weight of active compound, the remainder of the composition being pharmaceutically acceptable carriers, diluents or solvents.

Suitable pharmaceutically acceptable carriers include solid fillers or diluents and sterile aqueous or organic solutions. The active ingredient will be present in such pharmaceutical compositions in the amounts sufficient to provide the desired dosage in the range as described above. Thus, for oral administration, the active ingredient can be combined with a suitable solid or liquid carrier or diluent to form capsules, tablets, powders, syrups, solutions, suspensions and the like. The pharmaceutical compositions, may, if desired, contain additional components such as flavourants, sweeteners, excipients and the like. For parenteral administration, the active ingredient can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable acid addition salts or salts with base of the compounds. Aqueous solutions with the active ingredient dissolved in polyhydroxylated castor oil may also be used for injectable solutions. The injectable solutions prepared in this manner can then be administered intravenously, intraperitoneally, subcutaneously, or intramuscularly, with intramuscular administration being preferred in humans.

For nasal administration, the preparation may contain the active ingredient of the present invention dissolved or suspended in a liquid carrier, in particular an aqueous carrier, for aerosol application. The carrier may contain additives such as solubilizing agents, such as propylene glycol, surfactants, absorption enhancers such as lecithin (phosphatidylcholine) or cyclodextrin or preservatives such as parabenes.

Tablets, dragees or capsules having talc and/or a carbohydrate carried binder and the like are particularly suitable for any oral application. Preferably, carriers for tablets, dragees or capsules include lactose, corn starch and/or potato starch. A syrup or elixir can be used in cases where a sweetened vehicle can be employed.

A typical tablet production method is exemplified below:

Tablet Production Example:

| | | |
|---|---|---|
| a) | 1) Active ingredient | 30 g |
| | 2) Lactose | 95 g |
| | 3) Corn starch | 30 g |
| | 4) Carboxymethyl cellulose | 44 g |
| | 5) Magnesium stearate | 1 g |
| | | 200 g for 1000 tablets |

The ingredients 1 to 3 are uniformly blended with water and granulated after drying under reduced pressure. The ingredient 4 and 5 are mixed well with the granules and compressed by a tabletting machine to prepare 1000 tablets each containing 30 mg of active ingredient.

| | | | |
|---|---|---|---|
| b) | 1) Active ingredient | 30 | g |
| | 2) Calcium phosphate | 90 | g |
| | 3) Lactose | 40 | g |
| | 4) Corn starch | 35 | g |
| | 5) Polyvinyl pyrrolidone | 3.5 | g |
| | 6) Magnesium stearate | 1.5 | g |

200 g for 1000 tablets

The ingredients 1-4 are uniformly moistened with an aqueous solution of 5 and granulated after drying under reduced pressure. Ingredient 6 is added and granules are compressed by a tabletting machine to prepare 1000 tablets containing 30 mg of ingredient 1.

The compound of the formula (I) as defined above are clinically administered to mammals, including man, via either oral, nasal, pulmonary, transdermal or parenteral, rectal, depot, subcutaneous, intravenous, intraurethral, intramuscular, intranasal, ophthalmic solution or an ointment. Administration by the oral route is preferred, being more convenient and avoiding the possible pain and irritation of injection. However, in circumstances where the patient cannot swallow the medication, or absorption following oral administration is impaired, as by disease or other abnormality, it is essential that the drug be administered parenterally. By either route, the dosage is in the range of about 0.01 to about 100 mg/kg body weight of the subject per day or preferably about 0.01 to about 30 mg/kg body weight per day administered singly or as a divided dose. However, the optimum dosage for the individual subject being treated will be determined by the person responsible for treatment, generally smaller doses being administered initially and thereafter increments made to determine the most suitable dosage.

The invention is explained in detail in the examples given below which are provided by way of illustration only and therefore should not be construed to limit the scope of the invention.

(−)-3-[4-[2-(2-Ethyl-4-oxo-3,4-dihydroquinazolin-3-yl)ethoxy]phenyl]-2-ethoxypropanoic acid was prepared according to the procedure given in WO 99/08501 as given below:

A solution of [(2S)-N(1S)]-2-ethoxy-3-[4-[2-(2-Ethyl-4-oxo-3,4-dihydroquinazolin-3-yl)ethoxy]phenyl]-2-ethoxypropanoic acid-N-(2-hydroxy-1-phenylethyl)propanamide (267 mg, 0.504 mmol) in a mixture of 1M sulphuric acid and dioxane/water was heated at 100° C. for 16 h. The reaction mixture was cooled to ca 25° C. and dioxane was removed under reduced pressure. The remaining solution was cooled in an ice bath and the white solid precipitated was filtered and dried to afford (−)-3-[4-[2-(2-ethyl-4-oxo-3,4-dihydroquinazolin-3-yl)ethoxy]phenyl]-2-ethoxypropanoic acid (170 mg, 82%).

However, any other procedure for preparing (−)-3-[4-[2-(2-Ethyl-4-oxo-3,4-dihydroquinazolin-3-yl)ethoxy]phenyl]-2-ethoxypropanoic acid can be used.

(±)3-[4-[2-(2-Ethyl-4-oxo-3,4-dihydroquinazolin-3-yl)ethoxy]phenyl]-2-ethoxypropanoic acid and (+)-3-[4-[2-(2-Ethyl-4-oxo-3,4-dihydroquinazolin-3-yl)ethoxy]phenyl]-2-ethoxypropanoic acid can be prepared by a similar procedure described above or any procedure for making these compounds can be used.

### EXAMPLE-1

### (−)-3-[4-[2-(2-Ethyl-4-oxo-3,4-dihydroquinazolin-3-yl)ethoxy]phenyl]-2-ethoxypropanoic acid phenyl glycinol salt

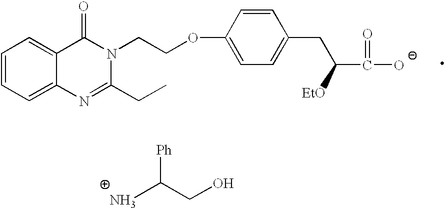

(−)-3-[4-[2-(2-Ethyl-4-oxo-3,4-dihydroquinazolin-3-yl)ethoxy]phenyl]-2-ethoxypropanoic acid (2 g) and isopropanol (50 ml) was added to 250 ml four necked round bottom flask fitted with a mechanical stirrer and reflux condenser. The reaction mixture was slowly heated to 45-55° C. for complete dissolution of the reaction mass. Phenyl glycinol (0.667 g) dissolved in isopropanol (10 ml) was added to the reaction mixture at 45-55° C. in about 10 min. under stirring. The progress of the reaction was maintained by gentle reflux of reaction mixture at 75-85° C. for 10 h. The reaction mixture was cooled to room temperature and stirred for 5 h at room temperature. The precipitated product was filtered, dried at 60° C. for 2-3 h to afford pure phenyl glycinol salt of (−)-3-[4-[2-(2-ethyl-4-oxo-3,4-dihydroquinazolin-3-yl)ethoxy]phenyl]-2-ethoxypropanoic acid as free flowing white crystalline solid, (weighs about 2 g, yield 75%, mp: 105-107° C., purity 99% by HPLC).

IR (KBr) cm⁻¹: 3400-3300 (O—H stretch), 2922 (—C—H aliphatic stretch), 1670 (—COO⁻ stretch), 1590(—CONH stretch), 1420 (—COO stretch).

¹H NMR (200 MHz, DMSO-d₆)δ: 8.13 (d, J=7.89 Hz, 1H), 7.82 (t, J=7.01 Hz, 1H), 7.64 (d, J=8.21 Hz, 1H), 7.50 (t, J=7.26 Hz, 2H), 7.4 (s, 5H), 7.13 (d, J=8.50 Hz, 2H), 6.84 (d, J=8.50 Hz, 2H), 4.47 (t, J=5.19 Hz, 2H), 4.4 (d, 2H), 4.26 (t, J=5.19 Hz, 2H), 3.99-3.84 (m, 1H), 3.60-3.40 (m, 1H), 3.40-3.20 (m, 2H), 3.06 (q, J=6.96 Hz, 2H), 2.88 (q, J=6.64 Hz, 2H), 1.32 (t, J=7.17 Hz, 3H), 1.02 (t, J=6.96 Hz, 3H).

Mass m/z: 411 (M⁺+1), 137 (C₈H₁₁NO). Anal. Calcd: C₃₁H₃₇N₃O₆; % C: 68.00; % H 6.76; % N 7.67, Found % C 67.80; % H 6.56; % N 7.57.

### EXAMPLE 2

### (−)-3-[4-[2-(2-Ethyl-4-oxo-3,4-dihydroquinazolin-3-yl)ethoxy]phenyl]-2-ethoxypropanoic acid R-(+) methyl benzylamine salt

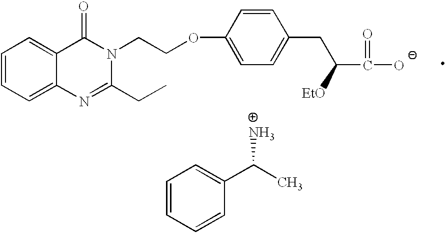

(−)-3-[4-[2-(2-Ethyl-4-oxo-3,4-dihydroquinazolin-3-yl)ethoxy]phenyl]-2-ethoxypropanoic acid (2.0 g) and isopropanol (50 ml) was added to 250 ml four necked round bottom flask, fitted with a mechanical stirrer and reflux condenser. The reaction mixture was slowly heated to 45-55° C. for complete dissolution of the reaction mass. R-(+) methyl benzylamine (0.589 g) in isopropanol (10 ml) was added to the reaction mixture at 45-55° C. in about 10 min. under stirring. The progress of the reaction was maintained by gentle reflux of reaction mixture at 75-85° C. for 10 h. The reaction mixture was cooled to room temperature and stirred for 12 h at room temperature. The reaction mixture was cooled to −5° C. and maintained at that temperature for 2 h under stirring. The precipitated product was filtered, dried at 60° C. for 2 h to afford pure (R)-(+) -methyl benzylamine salt of (−)-3-[4-[2-(2-ethyl-4-oxo-3,4-dihydroquinazolin-3-yl)ethoxy]phenyl]-2-ethoxypropanoic acid as free flowing off white amorphous solid, (weighs about 2 g, yield 80%, mp: 137-139° C., purity 99% by HPLC).

IR (KBr) cm⁻¹: 3400-3300 (N—H stretch), 3120 (—C—H aromatic), 2930 (—C—H aliphatic), 1660 (—COO stretch), 1583 (—CONH stretch), 1400 (—COO stretch).

¹H NMR (200 MHz, DMSO-d₆): 8.13 (d, J=7.89 Hz, 1H), 7.82 (t, J=7.01 Hz, 1H), 7.64 (d, J=8.21 Hz, 1H), 7.6-7.2 (m, 5H), 7.50 (t, J=7.26 Hz, 2H), 7.13 (d, J=8.50 Hz, 2H), 6.84 (d, J=8.50 Hz, 2H), 4.47 (t, J=5.19 Hz, 2H), 4.26 (t, J=5.19 Hz, 2H), 3.99-3.84 (m, 1H), 3.60-3.40 (m, 1H), 3.40-3.20 (m, 1H), 3.06 (q, J=6.96 Hz, 2H), 2.88 (q, J=6.64 Hz, 2H), 1.32 (t, J=7.17 Hz, 3H), 1.02 (t, J=6.96 Hz, 3H), 1.0 (d, 3H).

Mass m/z: 411 (M⁺+1), 121 (C₈H₁₁N). Anal. Calcd. C₃₁H₃₇N₃O₅, % C 70.05, % H 6.96; % N 7.90%; Found % C 69.82;% H 6.80; % N 7.70.

### EXAMPLE-3

### (−)-3-[4-[2-(2-Ethyl-4-oxo-3,4-dihydroquinazolin-3-yl)ethoxy]phenyl]-2-ethoxypropanoic acid dicyclohexylamine salt

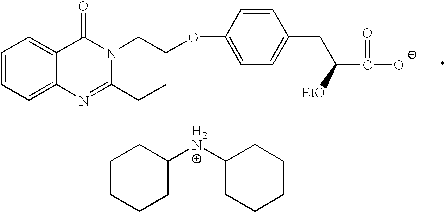

(−)-3-[4-[2-(2-Ethyl-4-oxo-3,4-dihydroquinazolin-3-yl)ethoxy]phenyl]-2-ethoxypropanoic acid (2 g) and isopropanol (50 ml) was added to 250 ml four necked round bottom flask, fitted with a mechanical stirrer and reflux condenser. The reaction mixture was slowly heated to 45-55° C. for complete dissolution of the mass. Dicyclohexylamine (0.88 g) dissolved in isopropanol (10 ml) was added to the reaction mixture at 45-55° C. in about 10 min. under stirring. The progress of the reaction was maintained by gentle reflux of reaction mixture at 75-85° C. for 10 h. The reaction mixture was cooled to 0-5° C. and maintained for 2 h under stirring. The precipitated product was filtered, dried at 60° C. for 2-3 h to afford pure dicyclohexylamine salt of (−)-3-[4-[2-(2-ethyl-4-oxo-3,4-dihydroquinazolin-3-yl)ethoxy]phenyl]-2-ethoxypropanoic acid as free flowing off-white crystalline solid, (weighs about 2.2 g, yield 76% mp: 152-153° C.; purity 99% by HPLC).

IR (KBr) cm⁻¹: 3400-3300 (—N—H stretch), 3100 (C—H, aromatic), 2930 (—C—H, aliphatic), 1670 (—COO stretch), 1597 (—CONH stretch), 1400 (—COO stretch).

¹H NMR (200 MHz, DMSO-d₆) 8.13 (d, J=7.89 Hz, 1H), 7.82 (t, J=7.01 Hz, 1H), 7.64 (d, J=8.21 Hz, 1H), 7.50 (t, J=7.26 Hz, 2H), 7.13 (d, J=8.50 Hz, 2H), 6.84 (d, J=8.50 Hz, 2H), 4.47 (t, J=5.19 Hz, 2H), 4.26 (t, J=5.19 Hz 2H), 3.99-3.84 (m, 1H), 3.60-3.40 (m, 1H), 3.40-3.20 (m, 1H), 3.06 (q, J=6.96 Hz, 2H), 2.88 (q, J=6.64 Hz, 2H), 2.6-0.8 (m, 22H), 1.32 (t, J=7.17 Hz, 3H), 1.02 (t, J=6.96 Hz, 3H).

Mass m/z: 411 (M⁺+1), 181 (C₁₂H₂₃N). Anal. Calcd: C₃₅H₄₉N₃O₅; % C: 71.06; % H 8.29; % N 7.10; Found % C 70.89;% H 8.10; % N 6.95.

### EXAMPLE 4

### (−)-3-[4-[2-(2-Ethyl-4-oxo-3,4-dihydroquinazolin-3-yl)ethoxy]phenyl]-2ethoxypropanoic acid lysine salt

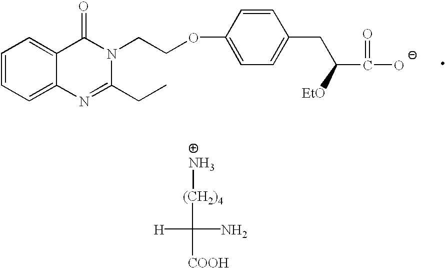

(−)-3-[4-[2-(2-Ethyl-4-oxo-3,4-dihydroquinazolin-3-yl)ethoxy]phenyl]-2-ethoxypropanoic acid (2 g) and isopropanol (50 ml) was added to 250 ml four necked round bottom flask, fitted with a mechanical stirrer and reflux condenser. The reaction mixture was slowly heated to 45-55° C. for complete dissolution of the mass. Lysine (0.8 g) dissolved in isopropanol (10 ml) was added to the reaction mixture at 45-55° C. in about 10 min. under stirring. The progress of the reaction was maintained by the gentle reflux of reaction mixture at 75-85° C. for 10 h. The reaction mixture was cooled to room temperature and stirred for 12 h at room temperature. The precipitated product was filtered, dried at 60° C. for 2-3 h to afford pure lysine salt of (−)-3-[4-[2-(2-ethyl-4-oxo-3,4-dihydroquinazolin-3-yl)ethoxy]phenyl]-2-ethoxypropanoic acid as free flowing off white amorphous solid (weights about 2 g, yield: 75%, mp: 152-155° C. purity 99% by HPLC).

IR (KBr) cm⁻¹: 3400-3300 (N—H stretch), 3120 (—C—H aromatic), 2930 (—C—H aliphatic), 1670 (—COO stretch), 1584 (—CONH stretch), 1407 (—COO stretch).

¹H NMR (200 MHz, DMSO-d₆)δ: 8.13 (d, J=7.89 Hz, 1H), 7.82 (t, J=7.01 Hz, 1H), 7.64 (d, J=8.21 Hz, 1H), 7.50 (t, J=7.26 Hz, 2H), 7.13 (d, J=8.50 Hz, 2H), 6.84 (d, J=8.50 Hz, 2H), 4.47 (t, J=5.19 Hz, 2H), 4.26 (t, J=5.19 Hz, 2H), 3.99-3.84 (m, 1H), 3.60-3.40 (m, 1H), 3.40-3.20 (m, 1H), 3.19 (t, 2H), 3.06 (q, J=6.96 Hz, 2H), 2.88 (q, J=6.64 Hz, 2H), 2.63 (t, 2H), 1.52 (m, 2H), 1.32 (t, J=7.17 Hz, 3H), 1.32 (m, 2H), 1.02 (t, J=6.96 Hz, 3H).

Mass m/z: 411 (M⁺+1), 146 (C₆H₁₄N₂O₂). Anal. Calcd: C₂₉H₄₀N₄O₇; % C 62.5; % H 7.19; % N 10.07; Found % C62.35; % H 7.10; % N 9.89.

### EXAMPLE 5

### (−)-3-[4-[2-(2-Ethyl-4-oxo-3,4-dihydroquinazolin-3-yl)ethoxy]phenyl]-2-ethoxypropanoic acid tris (hydroxymethyl)amino methane salt

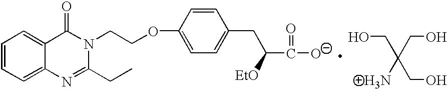

(−)-3-[4-[2-(2-Ethyl-4-oxo-3,4-dihydroquinazolin-3-yl)ethoxy]phenyl]-2-ethoxypropanoic acid (2 g) and isopropanol (50 ml) was added to 250 ml four necked round bottom flask, fitted with a mechanical stirrer and reflux condenser. The reaction mixture was slowly heated to 45-55° C. for complete dissolution of the mass. Tris (hydroxymethyl) amino methane (0.59 g) dissolved in isopropanol (10 ml) was added to the reaction mixture at 45-55° C. in about 10 min. under stirring. The progress of the reaction was maintained by gentle reflux of reaction mixture at 75-85° C. for 10 h. The reaction mixture was cooled to room temperature and stirred for 12 h. Distill off the isopropanol on rotavapor bath at 45-55° C. under vacuum. The product could not be isolated as a fine solid, because of hygroscopic nature and obtained as a sticky gummy mass, (weighs about 2 g, yield 77%, purity 99% by HPLC).

IR (KBr) cm⁻¹: 3400-3300 (N—H stretch), 3100 (C—H aromatic), 2937 (—C—H aliphatic), 1670 (—COO stretch), 1584 (—CONH stretch), 1395 (—COO stretch).

¹H NMR (200 MHz, DMSO-d₆) δ: 8.13 (d, J=7.89 Hz, 1H), 7.82 (t, J=7.01 Hz, 1H), 7.64 (d, J=8.21 Hz, 1H), 7.50 (t, J=7.26 Hz, 2H), 7.13 (d, J=8.50 Hz, 2H), 6.84 (d, J=8.50 Hz, 2H), 4.47 (t, J=5.19 Hz, 2H), 4.26 (t, J=5.19 Hz, 2H), 3.99-3.84 (m, 1H), 3.8 (s, 6H), 3.60-3.40 (m, 1H), 3.40-3.20 (m, 1H), 3.06 (q, J=6.96 Hz, 2H), 2.88 (q, J=6.64 Hz, 2H), 1.32 (t, J=7.17 Hz, 3H), 1.02 (t, J=6.96 Hz, 3H).

Mass m/z: 411 (M++1), 121 (C₄H₁₁NO₃).

### EXAMPLE 6

### (−)-3-[4-[2-(2-Ethyl-4-oxo-3,4-dihydroquinazolin-3-yl)ethoxy]phenyl]-2-ethoxypropanoic acid N-octyl glucamine salt

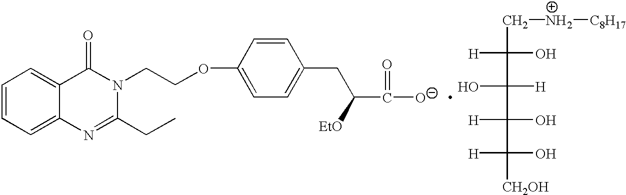

(−)-3-[4-[2-(2-Ethyl-4-oxo-3,4-dihydroquinazolin-3-yl)ethoxy]phenyl]-2-ethoxypropanoic acid (2 g) and isopropanol (50 ml) was added to 250 ml four necked round bottom flask, fitted with a mechanical stirrer and reflux condenser. The reaction mixture was slowly heated to 45-55° C. for complete dissolution of the mass. N-octyl glucamine salt (1.42 g) dissolved in isopropanol (10 ml) was added to the reaction mixture at 45-55° C. in about 10 min. under stirring. The progress of the reaction was maintained by the gentle reflux of reaction mixture at 75-85° C. for 6 h. The reaction mixture was cooled to room temperature and stirred for 12 h. The precipitated product was filtered and dried at 60° C. for 2-3 h to afford pure N-octyl glucamine salt (−)-3-[4-[2-(2-ethyl-4-oxo-3,4-dihydroquinazolin-3-yl)ethoxy]phenyl]-2-ethoxy propanoic acid as free flowing off white crystalline solid, (weighs about 2.7 g, yield 92%, mp: 114-116° C., purity 99% by HPLC).

IR (KBr) cm⁻¹: 3400-3250 (—N—H stretch), 2926 (—C—H stretch), 2800-2200 (—NH₃ stretch), 1776 (—COO stretch), 1593 (—CONH stretch), 1410 (—COO stretch).

¹H NMR (200 MHz, DMSO-d₆) δ: 8.13 (d, J=7.89 Hz, 1H), 7.82 (t, J=7.01 Hz, 1H), 7.64 (d, J=8.21 Hz, 1H), 7.50 (t, J=7.26 Hz, 2H), 7.13 (d, J=8.50 Hz, 2H), 6.84 (d, J=8.50 Hz, 2H), 4.47 (t, J=5.19 Hz, 2H), 4.26 (t, J=5.19 Hz, 2H), 4.2-3.06 (m, 8H), 3.99-3.84 (m, 1H), 3.60-3.40 (m, 1H), 3.40-3.20 (m, 1H), 3.06 (q, J=6.96 Hz, 2H), 2.88 (q, J=6.64 Hz, 2H), 1.5 (d, 3H), 1.40-1.00 (m, 16H), 1.32 (t, J=7.17 Hz, 3H), 1.02 (t, J=6.96 Hz, 3H).

Mass m/z: 411 (M⁺+1), 293 (C₁₄H₃₁NO₅). Anal. Calcd. C₃₇,H₅₇N₃O₁₀, % C 63.15; % H8.10; % N 5.97; Found %C 63.01; % H 7.91; % N 5.73.

### EXAMPLE 7

### (−)-3-[4-[2-(2-Ethyl-4-oxo-3,4-dihydroquinazolin-3-yl)ethoxy]phenyl]-2-ethoxypropanoic acid N-methyl glucamine salt

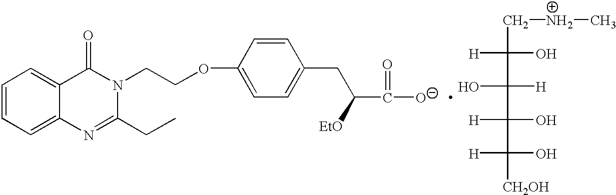

(−)-3-[4-[2-(2-Ethyl-4-oxo-3,4-dihydroquinazolin-3-yl)ethoxy]phenyl]-2-ethoxypropanoic acid (2 g) and isopropanol (50 ml) was added to 250 ml four necked round bottom flask, fitted with a mechanical stirrer and reflux condenser. The reaction mixture was slowly heated to 45-55° C. for complete dissolution of the mass. N-methyl glucamine (0.95 g) in isopropanol (20 ml) was added to the reaction mixture at 45-55° C. in about 10 min. under stirring. The progress of the reaction was maintained by gentle reflux of reaction mixture at 75-85° C. for 5 h. The reaction mixture was cooled to RT and stirred for 12 h at room temperature. The precipitated product was filtered, and dried at 60° C. for 2-3 h to afford pure N-methyl glucamine salt of (−)-3-[4-[2-(2-ethyl-4-oxo-3,4-dihydroquinazolin-3-yl)ethoxy]phenyl]-2-ethoxypropanoic acid as free flowing off white crystalline solid, (weighs about 2.75 g, yield: 93%, mp: 114-116° C., purity 99% by HPLC).

IR (KBr) cm⁻¹: 3350-3300 (—NH—OH stretching), 2960 (C—H stretch), 1670 (—COO stretch), 1887 (—CONH stretch).

¹H NMR (200 MHz, DMSO-d₆) δ: 8.13 (d, J=7.89 Hz, 1H), 7.82 (t, J=7.01 Hz, 1H), 7.64 (d, J=8.21 Hz, 1H), 7.50 (t, J=7.26 Hz, 2H), 7.13 (d, J=8.50 Hz, 2H), 6.84 (d, J=8.50 Hz, 2H), 4.47 (t, J=5.19 Hz, 2H), 4.26 (t, J=5.19 Hz, 2H), 4.0-3.26 (m, 8H), 3.99-3.84 (m, 1H), 3.60-3.40 (m, 1H), 3.40-3.20 (m, 1H), 3.06 (q, J=6.96 Hz, 2H), 2.88 (q, J=6.64 Hz, 2H), 2.4 (s, 3H), 1.32 (t, J=7.17 Hz, 3H), 1.02 (t, J=6.96 Hz, 3H).

Mass m/z: 411 (M⁺+1), 195 (C₇H₁₇NO═). Anal. Cacld. C₃₀H₄₃N₃O₁₀, % C 59.5; % H 7.10; % N 6.94; Found % C 59.25; % H6.9; % N 6.74.

### EXAMPLE 8

### (−)-3-[4-[2-(2-Ethyl-4-oxo-3,4-dihydroquinazolin-3-yl)ethoxy]phenyl]-2-ethoxypropanoic acid aminoguanidine hydrogen carbonate salt

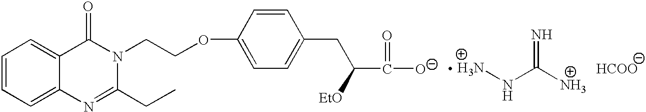

(−)-3-[4-[2-(2-Ethyl-4-oxo-3,4-dihydroquinazolin-3-yl)ethoxy]phenyl]-2-ethoxypropanoic acid (2 g) and isopropanol (50 ml) was added to 250 ml four necked round bottom flask, fitted with a mechanical stirrer and reflux condenser. The reaction mixture was slowly heated to 45-55° C. for complete dissolution of the mass. Aminoguanidine hydrogen carbonate (0.662 g) in isopropanol (20 ml) was added to the reaction mixture at 45-55° C. in about 10 min. under stirring. The progress of the reaction was maintained by gentle reflux of reaction mixture at 75-85° C. for 10 h. The reaction mixture was cooled to room temperature and stirred for 12 h at room temperature. Distill off the isopropanol on rotavapor water bath at 45-55° C. under stirring. The product could not be isolated as fine solid, because of hygroscopic nature and obtained as sticky gummy mass (weighs about 2 g, yield 75%, purity 99% by HPLC).

IR (KBr) cm⁻¹: 3400-3300 (N—H stretch), 2970 (—C—H, aliphatic stretch), 1675 (—COO stretch), 1595 (—CONH stretch), 1392 (—COO stretch).

¹H NMR (200 MHz, DMSO-d₆) δ: 8.13 (d, J=7.89 Hz, 1H), 7.82 (t, J=7.01 Hz, 1H), 7.64 (d, J=8.21 Hz, 1H), 7.50 (t, J=7.26 Hz, 2H), 7.13 (d, J=8.50 Hz, 2H), 6.84 (d, J=8.50 Hz, 2H), 4.47 (t, J=5.19 Hz, 2H), 4.26 (t, J=5.19 Hz, 2H), 3.99-3.84 (m, 1H), 3.60-3.40 (m, 1H), 3.40-3.20 (m, 1H), 3.06 (q, J=6.96 Hz, 2H), 2.88 (q, J=6.64 Hz, 2H), 2.4 (s, 6H), 1.32 (t, J=7.17 Hz, 3H), 1.02 (t, J=6.96 Hz, 3H).

Mass n/z: 411 (M⁺+1), 136 (C₂H₈N₄O₂).

### EXAMPLE 9

### (−)-3-[4-[2-(2-Ethyl-4-oxo-3,4-dihydroquinazolin-3-yl)ethoxy]phenyl]-2-ethoxypropanoic acid lithium salt

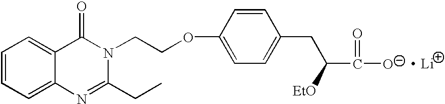

(−)-3-[4-[2-(2-Ethyl-4-oxo-3,4-dihydroquinazolin-3-yl)ethoxy]phenyl]-2-ethoxypropanoic acid (5 g) and isopropanol (100 ml) was added to 250 ml four necked round bottom flask, fitted with a mechanical stirrer and reflux condenser. The reaction mixture was slowly heated to 45-55° C. for complete dissolution of the mass. Lithium hydroxide hydrate (0.51 g) dissolved in water (20 ml) was added to the reaction mixture and heated to 75-85° C. for 12 h and monitor the progress of the reaction. The reaction mixture was cooled to room temperature and stirred for 12 h at room temperature. Distill off the isopropanol on rotavapor water bath at 45-55° C. under vacuum. Isopropanol (50 ml) was added and the precipitated product was filtered, dried at 60° C. for 2-3 to afford pure lithium salt of (−)-3-[4-[2-(2-ethyl-4-oxo-3,4-dihydroquinazolin-3-yl)ethoxy]phenyl]-2-ethoxypropanoic acid as off white crystalline solid, (weighs about 4.5 g, yield: 90%, mp: 245-47° C., purity 99% by HPLC).

IR (KBr) cm⁻¹: 3120 (—C—H aromatic), 2930 (—C—H aliphatic), 1660 (—COO stretch), 1587 (—CONH stretch), 1400 (—COO stretch).

¹H NMR (200 MHz, DMSO-d₆) δ: 8.13 (d, J=7.89 Hz, 1H), 7.82 (t, J=7.01 Hz, 1H), 7.64 (d, J=8.21 Hz, 1H), 7.50 (t, J=7.26 Hz, 2H), 7.13 (d, J=8.50 Hz, 2H), 6.84 (d, J=8.50 Hz, 2H), 4.47 (t, J=5.19 Hz, 2H), 4.26 (t, J=5.19 Hz, 2H), 3.99-3.84 (m, 1H), 3.60-3.40 (m, 1H), 3.40-3.20 (m, 1H), 3.06 (q, J=6.96 Hz, 2H), 2.88 (q, J=6.64 Hz, 2H), 1.32 (t, J=7.17 Hz, 3H), 1.02 (t, J=6.96 Hz, 3H).

Mass m/z: 411 (M⁺+1). Anal. Calcd: C₂₃H₂₅N₂O₅Li; % C 66.34; % H 6.00; % N 6.73; Found % C 66.12; % H 5.99; % N 6.53.

### EXAMPLE 10

### (−)-3-[4-[2-(2-Ethyl-4-oxo-3,4-dihydroquinazolin-3-yl)ethoxy]phenyl]-2-ethoxypropanoic acid magnesium salt

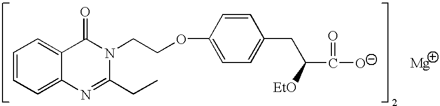

(−)-3-[4-[2-(2-Ethyl-4-oxo-3,4-dihydroquinazolin-3-yl)ethoxy]phenyl]-2-ethoxypropanoic acid (6 g) and isopropyl alcohol (60 ml) was added to 250 ml four necked round bottom flask, fitted with a mechanical stirrer and reflux condenser. The reaction mixture was slowly heated to 45-55° C. for complete dissolution of the mass. Magnesium hydroxide (0.423 g) was added to the reaction mixture and heated to 75-85° C. for 12 h and monitored the progress of the reaction. The reaction mixture was cooled to RT and stirred for 12 h at room temperature. Distill off the isopropanol on rotavapor water bath at 45-55° C. under vacuum and added isopropanol (5 ml) to the glassy residue and stirred for 10 min. The precipitated product was filtered, dried at 60° C. for 2-3 h to afford pure magnesium salt of (−)-3-[4-[2-(2-ethyl-4-oxo-3,4-dihydroquinazolin-3-yl)ethoxy]phenyl]-2-ethoxypropanoic acid as free flowing off white amorphous solid, (weighs about 5 g, yield: 80%, mp: >250° C., purity 99% by HPLC).

IR (KBr) cm⁻¹: 3120 (—C—H aromatic), 2930 (—C—H aliphatic), 1660 (—COO stretch), 1587 (—CONH stretch), 1400 (—COO stretch).

¹H NMR (200 MHz, DMSO-d₆) δ: 8.13 (d, J=7.89 Hz, 1H), 7.82 (t, J=7.01 Hz, 1H), 7.64 (d, J=8.21 Hz, 1H), 7.50 (t, J=7.26 Hz, 2H), 7.13 (d, J=8.50 Hz, 2H), 6.84 (d, J=8.50 Hz, 2H), 4.47 (t, J=5.19 Hz, 2H), 4.26 (t, J=5.19 Hz, 2H), 3.99-3.84 (m, 1H), 3.60-3.40 (m, 1H), 3.40-3.20 (m, 1H), 3.06 (q, J=6.96 Hz, 2H), 2.88 (q, J=6.64 Hz, 2H), 1.32 (t, J=7.17 Hz, 3H), 1.02 (t, J=6.96 Hz, 3H).

Mass m/z: 411 (M⁺+1). Anal. Calcd: C₄₆H₅₀N₄O₁₀Mg; % C 65.63; % H 5.94; % N 6.65; Found % C 65.50; % H 5.75; % N 6.50.

### EXAMPLE 11

### Form I

### (−)-3-[4-[2-(2-Ethyl-4-oxo-3,4-dihydroquinazolin-3-yl)ethoxy]phenyl]-2-ethoxypropanoic acid arginine salt

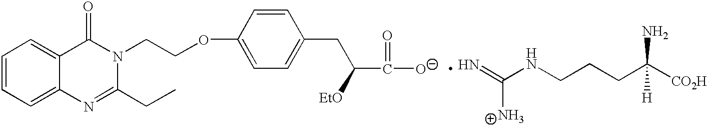

(−)-3-[4-[2-(2-Ethyl-4-oxo-3,4-dihydroquinazolin-3-yl)ethoxy]phenyl]-2-ethoxy propanoic acid (4.1 g), isopropanol (50 ml) was added to 250 ml four necked round bottom flask, fitted with a mechanical stirrer and reflux condenser. The reaction mixture was slowly heated to 45-55° C. in about 10 min. under stirring. L-Arginine (1.74 g) was dissolved in DM (demineralized) water (5 ml) and added at 45-55° C. under stirring. Maintained the gentle reflux of reaction mixture at 75-85° C. for 10 h and monitored the progress of the reaction. The reaction mixture was cooled to room temperature and stirred for 12 h at room temperature. Distill off the isopropanol on rotavapor bath at 45-55° C. under vacuum and added isopropanol (5 ml) to the glassy residue and stirred for 10 min. The precipitated product was filtered, dried at 60° C. for 2-3 h to afford form I of pure arginine salt of (−)-3-[4-[2-(2-ethyl-4-oxo-3,4-dihydroquinazolin-3-yl)ethoxy]phenyl]-2-ethoxy propanoic acid as off white amorphous solid (weights about 4.67 g, yield: 80%, mp: 215° C., purity 99% by HPLC).

IR (KBr) cm⁻¹: 3400-3300 (N—H stretch), 3120 (—C—H, aromatic), 2930 (—C—H aliphatic), 1670 (—COO stretch(, 1584 (—CONH stretch), 1407 (—COO stretch).

¹H NMR (200 MHz, DMSO-d₆) δ: 8.13 (d, J=7.89 Hz, 1H), 7.82 (t, J=7.01 Hz, 1H), 7.64 (d, J=8.21 Hz, 1H), 7.50 (t, J=7.26 Hz, 2H), 7.13 (d, J=8.50 Hz, 2H), 6.84 (d, J=8.50 Hz, 2H), 4.47 (t, J=5.19 Hz, 2H), 4.26 (t, J=5.19 Hz, 2H), 3.99-3.84 (m, 1H), 3.8-3.2 (m, 7H), 3.60-3.40 (m, 1H), 3.40-3.20 (m, 1H), 3.19 (t, 2H), 3.06 (q, J=6.96 Hz, 2H), 2.88 (q, J=6.64 Hz, 2H), 2.63 (t, 2H), 1.52 (m, 2H), 1.32 (t, J=7.17 Hz, 3H), 1.32 (m, 2H), 1.02 (t, J=6.96 Hz, 3H).

Mass m/z: 411 (M⁺+1). Anal. Calcd: C₂₉H₄₀N₆O₇; % C 59.57; % H 6.84; % N 14.38; Found % C 59.40; % H 6.00; % N 13.28.

### Form II

(−)-2-Ethoxy-3-[4-[2-(2-ethyl-4-oxo-3,4-dihydro-3-quinazolinyl)ethoxy]phenyl]propionic acid (140 g), isopropanol (2.8 L) was added to 5 L four necked round bottom flask, fitted with a mechanical stirrer and reflux condenser. The reaction mixture was slowly heated to 65-75° C. in about 10 min. under stirring. L-Arginine (59.2 g) dissolved in DM (demineralized) water (296 ml) was added at 65-75° C. under stirring. After addition of the arginine solution, the reaction mixture becomes clear and precipitation appeared immediately in the reaction mixture. Maintained the gentle reflux of reaction mixture at 75-85° C. for 4-8 h and monitored the progress of the reaction. The reaction mixture was cooled to room temperature and stirred for 2 h at room temperature. The precipitated product was filtered, dried at 60-65° C. for 5-6 h, till MC (moisture content) reached <1%, to afford form II of pure arginine salt of (−)-3-[4-[2-(2-ethyl-4-oxo-3,4-dihydroquinazolin-3-yl)ethoxy]phenyl]-2-ethoxypropanoic acid as white crystalline solid, (weights about 180 g, yield: 90%, mp 216-220° C., purity 99% by HPLC).

IR (KBr) cm⁻¹: 3400-3300 (N—H stretch), 3120 (—C—H aromatic), 2930 (—C—H aliphatic), 1712 (—COOH), 1670 (—COO stretch, —CONH stretch), 1584 (—CONH stretch), 1407 (—COO stretch).

¹H NMR (200 MHz, DMSO-d₆) δ: 8.13 (d, J=7.89 Hz, 1H), 7.82 (t, J=7.01 Hz, 1H), 7.64 (d, J=8.21 Hz, 1H), 7.50 (t, J=7.26 Hz, 2H), 7.13 (d, J=8.50 Hz, 2H), 6.84 (d, J=8.50 Hz, 2H), 4.47 (t, J=5.19 Hz, 2H), 4.26 (t, J=5.19 Hz, 2H), 3.99-3.84 (m, 1H), 3.8-3.2 (m, 7H), 3.60-3.40 (m, 1H), 3.40-3.20 (m, 1H), 3.19 (t, 2H), 3.06 (q, J=6.96 Hz, 2H), 2.88 (q, J=6.64 Hz, 2H), 2.63 (t, 2H), 1.52 (m, 2H), 1.32 (t, J=7.17 Hz, 3H), 1.32 (m, 2H), 1.02 (t, J=6.96 Hz, 3H).

Mass m/z: 411 (M⁺+1). Anal. Calcd: C₂₉H₄₀N₆O₇; % C 59.57; % H 6.84; % N 14.38; Found % C 59.40; % H 6.00; % N 13.28.

### EXAMPLE 12

### (−)-3-[4-[2-(2-Azido-4-oxo-3,4-dihydroquinazolin-3-yl)ethoxy]phenyl]-2-ethoxypropanoic acid magnesium salt

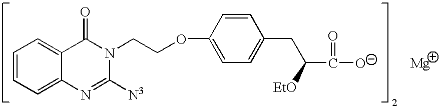

(−)-3-[4-[2-(2-Azo-4-oxo-3,4-dihydroquinazolin-3-yl)ethoxy]phenyl]-2-ethoxypropanoic acid (61 mg), dry methanol (5 ml) and magnesium hydroxide (3.98 mg) was added to 50 ml one necked round bottom flask, fitted with a mechanical stiffer and reflux condenser. The reaction mixture was refluxed at 80° C. for 12 h and monitored the progress of the reaction. The reaction mixture was cooled to RT and distill off the methanol. The residue was washed with dry ether and dried under vacuum to afford pure magnesium salt of (−)-3-[4-[2-(2-azo-4-oxo-3,4-dihydroquinazolin-3-yl)ethoxy]phenyl]-2-ethoxypropanoic acid a solid, (weighs about 61 mg, yield: 48.8%, mp: 230-235° C., purity 90.75% by HPLC).

IR (KBr) cm⁻¹: 3425, 2926, 1696, 1624, 1565.

¹H NMR (200 MHz, CDCl₃) δ: 8.39 (d, J=7.89 Hz, 1H), 8.29 (d, J=7.89 Hz, 1H), 8.05-7.95 (m, 1H), 7.8-7.7 (m, 1H), 7.10 (d, J=8.21 Hz, 2H), 6.74 (d, J=7.89 Hz, 2H), 4.4-4.3 (m, 4H), 3.8-3.7 (m, 1H), 3.6-3.5 (m, 1H), 3.4-3.2 (m, 1H), 3.0-2.8 (m, 1H), 2.8-2.7 (m, 1H), 1.06 (t, J=6.64 Hz, 3H).

### EXAMPLE 13

### (−)-3-[4-[2-(2-Morpholinyl-4-oxo-3,4-dihydroquinazolin-3-yl)ethoxy]phenyl]-2-ethoxypropanoic acid magnesium salt

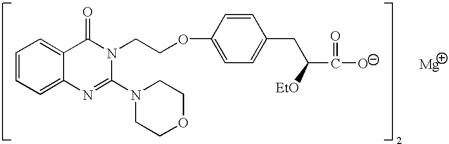

(−)-3-[4-[2-(2-Morpholinyl-4-oxo-3,4-dihydroquinazolin-3-yl)ethoxy]phenyl]-2-ethoxypropanoic acid (160 mg), dry methanol (5 ml) and magnesium hydroxide (9.45 mg) was added to 50 ml one necked round bottom flask, fitted with a mechanical stirrer and reflux condenser. The reaction mixture was refluxed at 80° C. for 12 h and monitored the progress of the reaction. The reaction mixture was cooled to RT and distill off the methanol. The residue was washed with dry ether and dried under vacuum to afford pure magnesium salt of (−)-3-[4-[2-(2-morpholinyl-4-oxo-3,4-dihydroquinazolin-3-yl)ethoxy]phenyl]-2-ethoxypropanoic acid a solid, (weighs about 100 mg, yield: 30.58%, mp: 250-255° C., purity 90.43% by HPLC).

IR (KBr) cm⁻¹: 3441, 2924, 2854, 1674, 1610, 1587.

¹H NMR (200 MHz, CDCl₃) δ: 8.16 (d, J=7.89 Hz, 1H), 7.71 (d, J=7.06 Hz, 1H), 7.60-7.50 (m, 1H), 7.50-7.35 (m, 1H), 7.16 (d, J=7.98 Hz, 2H), 6.75 (d, J=8.21 Hz, 2H), 4.56 (t, J=8.14 Hz, 3H), 4.40-4.30 (m, 3H), 3.90-3.65 (m, 4H), 3.65-3.50 (m, 2H), 3.40-3.10 (m, 5H), 3.05-2.90 (m, 1H), 2.90-2.70 (m, 1H), 1.15-1.00 (m, 3H).

### EXAMPLE 14

### (−)-3-[4-[2-(2-Piperidinyl-4-oxo-3,4-dihydroquinazolin-3-yl)ethoxy]phenyl]-2-ethoxypropanoic acid magnesium salt

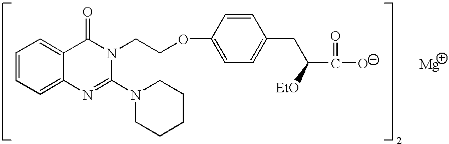

(−)-3-[4-[2-(2-Piperidinyl-4-oxo-3,4-dihydroquinazolin-3-yl)ethoxy]phenyl]-2-ethoxypropanoic acid (147 mg), dry methanol (5 ml) and magnesium hydroxide (8.72 mg) was added to 50 ml one necked round bottom flask, fitted with a mechanical stirrer and reflux condenser. The reaction mixture was refluxed at 80° C. for 12 h and monitored the progress of the reaction. The reaction mixture was cooled to RT and distill off the methanol. The residue was washed with dry ether and dried under vacuum to afford pure magnesium salt of (−)-3-[4-[2-(2-morpholinyl-4-oxo-3,4-dihydroquinazolin-3-yl]ethoxy)phenyl]-2-ethoxypropanoic acid as a solid, (weighs about 100 mg, yield: 33.24%, mp: 220-225° C., purity 95.30% by HPLC).

IR (KBr) cm⁻¹: 2933, 1674, 1610, 1584, 1566.

¹H NMR (200 MHz, CDCl₃) δ: 8.12 (d, J=7.80 Hz, 1H), 7.69 (t, J=4.00 Hz, 1H), 7.53 (t, J=8.40 Hz, 1H), 7.35 (t, J=7.50 Hz, 1H), 7.13 (d, J=8.20 Hz, 2H), 6.74 (d, J=8.00 Hz, 2H), 4.54 (t, J=4.90 Hz, 2H), 4.31 (t, J=5.20 Hz, 2H), 3.90-3.75 (m, 1H), 3.65-3.50 (m, 1H), 3.33-3.26 (m, 1H), 3.20-3.03 (m, 4H) 2.95-2.70 (m, 2H), 1.75-1.50 (m, 6H), 1.10-1.00 (m, 3H).

### EXAMPLE 15

### (−)-3-[4-[2-(2-Ethyl-4-oxo-3,4-dihydroquinazolin-3-yl)ethoxy]phenyl]-2-ethoxy propanoic acid metformin salt

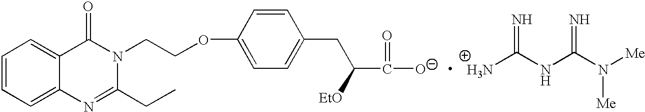

(−)-2-Ethoxy-3-[4-[2-(2-ethyl-4-oxo-3,4-dihydro-3-quinazolinyl)ethoxy]phenyl]propionic acid (2.0 g) and isopropanol (50 ml) was added to 250 ml four necked round bottom flask, fitted with a mechanical stirrer and reflux condenser. The reaction mixture was slowly heated to 45-55° for complete dissolution of the mass. Metformin (0.663 g) dissolved in isopropanol (10 ml) was added to the reaction mixture of 75-85° C. for 5 h and monitor the progress of the reaction. The reaction mixture was cooled to room temperature and stirred for 12 h at room temperature. Distill off the isopropanol on rotavapor water bath at 45-55° C. under vacuum. The precipitated product was filtered, dried at 60 ° C. for 2-3 h to afford the pure metformin salt of (−)-3-[4-[2-(2-ethyl-4-oxo-3,4-dihydroquinazolin-3-yl)ethoxy]phenyl-2-ethoxy propanoic acid as off white crystalline solid, (weighs about 2.0 g, Yield: 77%, m.p. 164-66° C., purity 99% by HPLC).

IR as KBr shows the following absorption bands (cm⁻¹) 3400-3300 (N—H stretch), 3120 (—C—H aromatic), 2930 (—C—H aliphatic), 1660 (—COO stretch), 1587 (—CONH stretch), 1400 (—COO stretch).

¹H NMR spectrum in DMSO-d₆ (TMS as internal standard) shows the following signals δ8.13 (d, J=7.89 Hz, 1H), 7.82 (t, J=7.01 Hz, 1H), 7.64(d, J=8.21 Hz, 1H), 7.50 (t, J=7.26 Hz, 2H), 7.13(d, J=8.50 Hz, 2H), 6.84 (d, J=8.50 Hz, 2H), 4.47 (t, J=5.19 Hz, 2H), 4.26 (t, J=5.19 Hz, 2H), 3.99-3.84 (m, 1H), 3.60-3.40 (m, 1H), 3.40-3.20 (m, 1H), 3.06 (q, J=6.96 Hz, 2H), 2.88 (q, J=6.64 Hz, 2H), 2.8 (s, 6H), 1.32 (t, J=7.17 Hz, 3H), 1.02 (t, J=6.96 Hz, 3H).

The mass spectrum shows m/z, 411 (M⁺+1), 130 (C₄H₁₁N₅), Anal. Calcd: C₂₃H₂₈N₂O₅; % C 60.11; % H 6.86; % N 18.18; Found % C 59.89; % H 6.66; % N 18.00.

### EXAMPLE-16

### 3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid magnesium salt

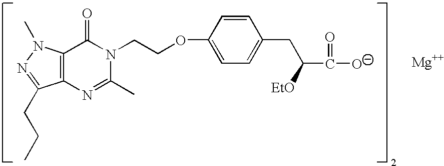

3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid (5.0 g), methanol (50 ml) were added to 250 ml four necked round bottom flask, fitted with a mechanical stirrer and reflux condenser. The reaction mixture was slowly heated to 55-60° C. for complete dissolution of the mass. Magnesium hydroxide (0.328 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum dried to afford the pure magnesium salt of 3-[4-[2-(1,5-dimethyl-7-oxo-3-propyl-6,7-dihydro-1H-pyrazolo[4,3-d]pyrimidin-6-yl)ethoxy]phenyl]-2-ethoxypropionic acid as off white crystalline solid, (weighs about 4.09 g, yield: 80%, mp 113° C., purity 99% by HPLC).

IR (KBr) cm⁻¹: 3120 (C—H aromatic), 2930 (—C—H aliphatic), 1693 (—COO/—CONH stretch), 1574 (—CONH amide-II band).

¹H NMR (200 MHz, DMSO-d₆) δ: 7.13 (d, J=8.3 Hz, 2H), 6.78 (d, J=8.3 Hz, 2H), 4.48 (t, J=4.6 Hz, 2H), 4.35-4.15 (m, 5H), 4.01-3.97 (m, 1H), 3.56-3.38 (m, 2H), 3.12-2.90 (m, 2H), 2.83 (t, J=7. Hz, 2H), 2.76 (s, 3H), 1.89-1.71 (m, 2H), 1.16 (t, J=7.0 Hz, 3H), 0.98 (t, J=7.3 Hz, 3H).

Mass m/z: 443 (M⁺+1), Anal. Calcd: C₄₆H₅₈N₈O₁₀Mg; % C 60.92; % H: 6.40; % N 12.31; Found % C 60.80; % H 6.30; % N 12.20.

### EXAMPLE-17

### 3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid potassium salt

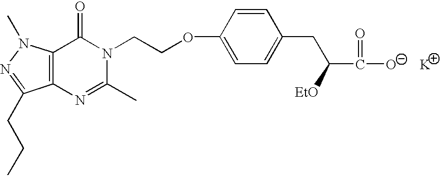

3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid (5.0 g), methanol (50 ml) were added to 250 ml four necked round bottom flask, fitted with a mechanical stirrer and reflux condenser. The reaction mixture was slowly heated to 55-60° C. for complete dissolution of the mass. Potassium hydroxide (0.63 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum-dried to afford the pure potassium salt of 3-[4-[2-(1,5-dimethyl-7-oxo-3-propyl-6,7-dihydro-1H-pyrazolo[4,3-d]pyrimidin-6-yl)ethoxy]phenyl]-2-ethoxypropionic acid as off-white crystalline solid (weighs about 4.6 g, yield: 85%, mp: 168-170° C., purity 99% by HPLC).

IR (KBr) cm⁻¹: 3120 (C—H aromatic), 2960 (—C—H aliphatic), 1690 (—COO/—CONH stretch), 1573 (—CONH amide-II band).

¹H NMR (200 MHz, DMSO-d₆) δ: 7.13 (d, J=8.3 Hz, 2H), 6.78 (d, J=8.3 Hz, 2H), 4.48 (t, J=4.6 Hz, 2H), 4.35-4.15 (m, 5H), 4.01-3.97 (m, 1H), 3.56-3.38 (m, 2H), 3.12-2.90 (m, 2H), 2.83 (t, J=7.8 Hz, 2H), 2.76 (s, 3H), 1.89-1.71 (m, 2H), 1.16 (t, J=7.0 Hz, 3H), 0.98 (t, J=7.3 Hz, 3H).

Mass m/z: 443 (M⁺+1), Anal. Calcd: C₂₃H₂₉N₄O₅K; % C 57.49; % H: 6.04; % N 11.66; Found % C 57.30; % H 5.9; % N 11.50.

### EXAMPLE-18

### 3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid calcium salt

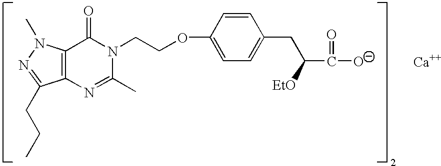

3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid (5.0 g), methanol (50 ml) were added to 250 ml four necked round bottom flask, fitted with a mechanical stirrer and reflux condenser. The reaction mixture was slowly heated to 55-60° C. for complete dissolution of the mass. Calcium hydroxide (0.418 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum-dried to afford the pure calcium salt of 3-[4-[2-(1,5-dimethyl-7-oxo-3-propyl-6,7-dihydro-1H-pyrazolo[4,3-d]pyrimidin-6-yl)ethoxy]phenyl]-2-ethoxypropionic acid as off white crystalline solid (weighs about 4.17 g, yield: 80%, mp: 251° C. (dec), purity 99% by HPLC).

IR (KBr) cm⁻¹: 3150 (C—H aromatic), 2960 (—C—H aliphatic), 1690 (—COO/—CONH stretch), 1577 (—CONH amide II band).

¹H NMR (200 MHz, DMSO-d₆) δ: 7.13 (d, J=8.3 Hz, 2H), 6.78 (d, J=8.3 Hz, 2H), 4.48 (t, J=4.6 Hz, 2H), 4.35-4.15 (m, 5H), 4.10-3.97 (m, 1H), 3.56-3.38 (m, 2H), 3.12-2.90 (m, 2H), 2.83 (t, J=7.8 Hz, 2H), 2.76 (s, 3H), 1.89-1.71 (m, 2H), 1.16 (t, J=7.0 Hz, 3H), 0.98 (t, J=7.3 Hz, 3H).

Mass m/z: 443 (M⁺+1), Anal. Calcd: C₄₆H₅₈N₈O₁₀Ca; % C 59.86; % H: 6.29; % N 12.14; Found % C 59.70; % H 6.15; % N 12.0.

### EXAMPLE-19

### 3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid lithium salt

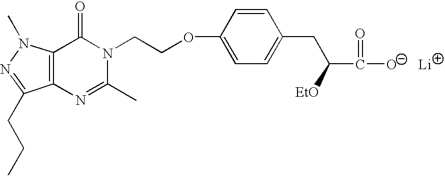

3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid (5.0 g), methanol (50 ml) were added to 250 ml four necked round bottom flask, fitted with a mechanical stirrer and reflux condenser. The reaction mixture was slowly heated to 55-60° C. for complete dissolution of the mass. Lithium hydroxide (0.47 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum-dried to afford the pure lithium salt of 3-[4-[2-(1,5-dimethyl-7-oxo-3-propyl-6,7-dihydro-1H-pyrazolo[4,3-d]pyrimidin-6-yl)ethoxy]phenyl]-2-ethoxypropionic acid as off-white crystalline solid (weighs about 4.3 g, yield: 85%, mp: 254-266° C., purity 99% by HPLC).

IR (KBr) cm⁻¹: 3150 (C—H aromatic), 2950 (—C—H aliphatic), 1690 (—COO/—CONH stretch), 1574 (—CONH amide-II band).

¹H NMR (200 MHz, DMSO-d₆) δ: 7.13 (d, J=8.3 Hz, 2H), 6.78 (d, J=8.3 Hz, 2H), 4.48 (t, J=4.6 Hz, 2H), 4.35-4.15 (m, 5H), 4.10-3.97 (m, 1H), 3.56-3.38 (m, 2H), 3.12-2.90 (m, 2H), 2.83 (t, J=7.8 Hz, 2H), 2.76 (s, 3H), 1.89-1.71 (m, 2H), 1.16 (t, J=7.0 Hz, 3H), 0.98 (t, J=7.3 Hz, 3H).

Mass m/z: 443 (M⁺+1), Anal. Calcd: C₂₃H₂₉N₄O₅Li; % C 61.60; % H: 6.47; % N 12.49; Found % C 61.45; % H 6.31; % N 12.35.

### EXAMPLE-20

### 3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid sodium salt

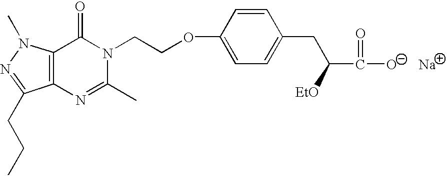

3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid (5.0 g), methanol (50 ml) were added to 250 ml four necked round bottom flask, fitted with a mechanical stirrer and reflux condenser. The reaction mixture was slowly heated to 55-60° C. for complete dissolution of the mass. Sodium hydroxide (0.45 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum-dried to afford the pure sodium salt of 3-[4-[2-(1,5-dimethyl-7-oxo-3-propyl-6,7-dihydro-1H-pyrazolo[4,3-d]pyrimidin-6-yl)ethoxy]phenyl]-2-ethoxypropionic acid as off white hygroscopic solid (weighs about 4.19 g, yield: 80%, purity 99% by HPLC).

IR (KBr) cm⁻¹: 3100 (C—H aromatic), 2963 (—C—H aliphatic), 1688 (—COO/—CONH stretch), 1574 (—CONH amide-II band).

¹H NMR (200 MHz, DMSO-d₆) δ: 7.13 (d, J=8.3 Hz, 2H), 6.78 (d, J=8.3 Hz, 2H), 4.48 (t, J=4.6 Hz, 2H), 4.35-4.15 (m, 5H), 4.10-3.97 (m, 1H), 3.56-3.38 (m, 2H), 3.12-2.90 (m, 2H), 2.83 (t, J=7.8 Hz, 2H), 2.76 (s, 3H), 1.89-1.71 (m, 2H), 1.16 (t, J=7.0 Hz, 3H), 0.98 (t, J=7.3 Hz, 3H).

Mass m/z: 443 (M⁺+1).

### EXAMPLE 21

### 3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid arginine salt

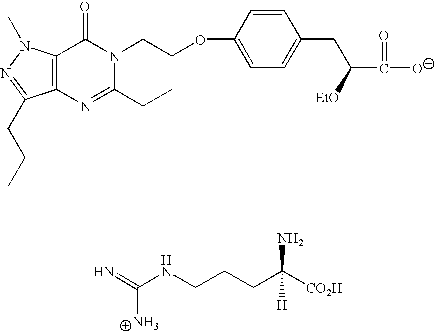

3-[4-[2-(1,5-Dimethyl-7-oxo-3-propyl -6,7-dihydro- 1H-pyrazolo[4,3-d]pyrimidin-6-yl)ethoxy]phenyl]-2-ethoxypropionic acid (5.0 g), isopropanol (50 ml) were added to 250 ml four necked round bottom flask, fitted with a mechanical stirrer and reflux condenser. The reaction mixture was slowly heated to 55-60° C. for complete dissolution of the mass. L-Arginine (1.96 g) was added to the reaction mixture at 60° in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum-dried to afford the pure arginine salt of 3-[4-[2-(1,5-dimethyl-7-oxo-3-propyl-6,7-dihydro-1H-pyrazolo[4,3-d]pyrimidin-6-yl)ethoxy]phenyl]-2-ethoxypropionic acid as off white crystalline solid (weighs about 5.92 g, yield: 85%, mp: 192° C., purity 99% by HPLC).

IR (KBr) cm⁻¹: 3300-3200 (—NH stretch), 3150 (C—H aromatic), 2950 (—C—H aliphatic), 1690 (—COO/—CONH stretch), 1583 (—CONH amide-II band).

¹H NMR (200 MHz, DMSO-d₆) δ: 7.13 (d, J=8.3 Hz, 2H), 6.78 (d, J=8.3 Hz, 2H), 4.48 (t, J=4.6 Hz, 2H), 4.35-4.15 (m, 5H), 4.10-3.97 (m, 1H), 3.80 (m, 2H), 3.56-3.38 (m, 2H), 3.20 (m, 2H), 3.12-2.90 (m, 2H), 2.83 (t, J=7.8 Hz, 2H), 2.76 (s, 3H), 2.0-1.6 (m, 3H), 1.89-1.71 (m, 2H), 1.16 (t, J=7.0 Hz, 3H), 0.98 (t, J=7.3 Hz, 3H).

Mass m/z: 443 (M⁺+1), Anal. Calcd: C₂₉H₄₄N₈O₇; % C 56.49; % H: 7.14; % N 18.18; Found % C 56.34; % H 6.98; % N 17.99.

### EXAMPLE-22

### 3-[4-[2-(1,5-Dimethyl-7-oxo-3-propyl-6,7-dihydro- 1H-pyrazolo[4,3-d]pyrimidin-6-yl)ethoxy]phenyl]-2-ethoxypropionic acid R-(+) methyl benzylamine salt

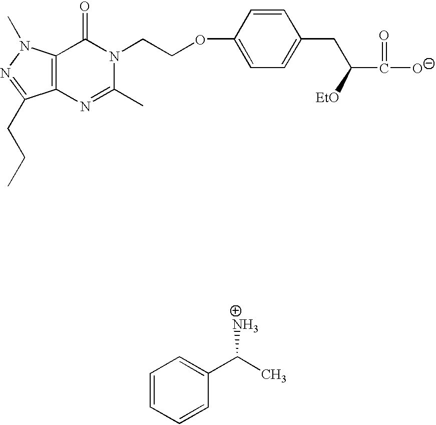

3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid (5.0 g), isopropanol (50 ml) were added to 250 ml four necked round bottom flask, fitted with a mechanical stirrer and reflux condenser. The reaction mixture was slowly heated to 55-60° C. for complete dissolution of the mass. R-(+) methyl benzylamine (1.36 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum-dried to afford the pure, hygroscopic 3-[4-[2-(1,5-dimethyl-7-oxo-3-propyl-6,7-dihydro-1H-pyrazolo[4,3-d]pyrimidin-6-yl)ethoxy]phenyl]-2-ethoxypropionic acid R-(+) methyl benzylamine salt as off white crystalline solid (weighs about 5.41 g, yield: 85%, mp: 114-115° C., purity 99% by HPLC).

IR (KBr) cm⁻¹: 3150 (C—H aromatic), 2940 (—C—H aliphatic), 1687 (—COO/—CONH stretch), 1573 (—CONH amide-II band).

¹H NMR (200 MHz, DMSO-d₆) δ: 7.40-7.20 (m, 5H), 7.13 (d, J=8.3 Hz, 2H), 6.78 (d, J=8.3 Hz, 2H), 4.48 (t, J=4.6 Hz, 2H), 4.35-4.15 (m, 5H), 4.10 (m, 1H), 4.10-3.97 (m, 1H), 3.56-3.38 (m, 2H), 3.12-2.90 (m, 2H), 2.83 (t, J=7.8 Hz, 2H), 2.76 (s, 3H), 1.89-1.71 (m, 2H), 1.40 (d, 3H), 1.16 (t, J=7.0 Hz, 3H), 0.98 (t, J=7.3 Hz, 3H).

Mass m/z: 443 (M⁺+1), Anal. Calcd: C₃₁H₄₁N₅O₅; % C 66.07; % H: 7.28; % N 12.43; Found % C 65.90; % H 7.15; % N 12.33.

### EXAMPLE-23

### 3-[4-[2-(1,5-Dimethyl-7-oxo-3-propyl-6,7-dihydro- 1H-pyrazolo[4,3-d]pyrimidin-6-yl)ethoxy]phenyl]-2-ethoxypropionic acid S-(+)-phenylglycinol salt

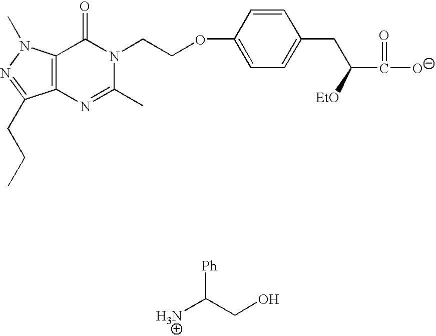

3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid (5.0 g), isopropanol (50 ml) were added to 250 ml four necked round bottom flask, fitted with a mechanical stirrer and reflux condenser. The reaction mixture was slowly heated to 55-60° C. for complete dissolution of the mass. S-(+)-Phenylglycinol (1.549 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum dried to afford the pure S-(+)-phenylglycinol salt of 3-[4-[2-(1,5-dimethyl-7-oxo-3-propyl-6,7-dihydro-1H-pyrazolo[4,3-d]pyrimidin-6-yl)ethoxy]phenyl]-2-ethoxypropionic acid of the formula as off white hygroscopic solid (weighs about 5.23 g, yield: 80%, purity 99% by HPLC).

IR (KBr) cm⁻¹: 3400-3300 (O—H stretch), 3150 (C—H aromatic), 2950 (—C—H aliphatic), 1689 (—COO/—CONH stretch), 1573 (—CONH amide-II band).

¹H NMR (200 MHz, DMSO-d₆) δ: 7.40 (m, 5H), 7.13 (d, J=8.3 Hz, 2H), 6.78 (d, J=8.3 Hz, 2H), 4.48 (t, J=4.6 Hz, 2H), 4.35-4.15 (m, 5H), 4.10-3.97 (m, 1H), 4.00 (d, 2H), 3.60-3.50 (m, 1H), 3.56-3.38 (m, 2H), 3.12-2.90 (m, 2H), 2.83 (t, J=7.8 Hz, 2H), 2.76 (s, 3H), 1.89-1.71 (m, 2H), 1.16 (t, J=7.0 Hz, 3H), 0.98 (t, J=7.3 Hz, 3H).

Mass m/z: 443 (M⁺+1).

### EXAMPLE 24

### 3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid aminoguanidine hydrogen carbonate salt

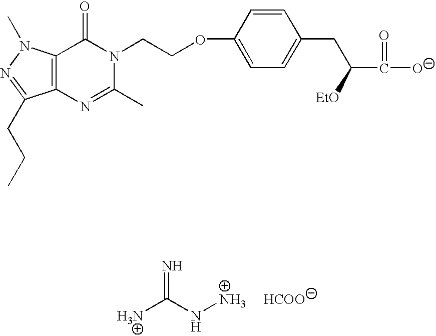

3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid (5.0 g), isopropanol (50 ml) were added to 250 ml four necked round bottom flask, fitted with a mechanical stirrer and reflux condenser. The reaction mixture was slowly heated to 55-60° C. for complete dissolution of the mass. 1.53 g of Aminoguanidine hydrogen carbonate was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum-dried to afford the pure aminoguanidine salt of 3-[4-[2-(1,5-dimethyl-7-oxo-3-propyl-6,7-dihydro-1H-pyrazolo[4,3-d]pyrimidin-6-yl)ethoxy]phenyl]-2-ethoxypropionic acid as off white hygroscope solid, (weighs about 5.5 g, yield: 85%, mp: 110-112° C., purity 99% by HPLC).

IR (KBr) cm⁻¹: 3450-3350 (—NH stretch), 3150 (C—H aromatic), 2956 (—C—H aliphatic), 1684 (—COO/—CONH stretch), 1572 (—CONH amide-II band).

¹H NMR (200 MHz, DMSO-d₆) δ: 7.13 (d, J=8.3 Hz, 2H), 6.78 (d, J=8.3 Hz, 2H), 4.48 (t, J=4.6 Hz, 2H), 4.35-4.15 (m, 5H), 4.10-3.97 (m, 1H), 3.56-3.38 (m, 2H), 3.12-2.90 (m, 2H), 2.83 (t, J=7.8 Hz, 2H), 2.76 (s, 3H), 1.89-1.71 (m, 2H), 1.16 (t, J=7.0 Hz, 3H), 0.98 (t, J=7.3 Hz, 3H).

Mass m/z: 443 (M⁺+1).

### EXAMPLE 25

### 3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid tromethamine salt

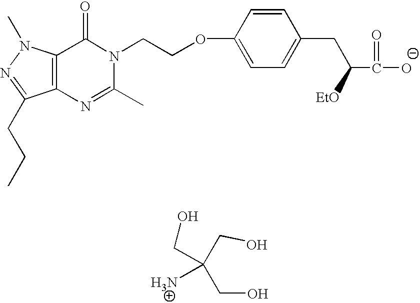

3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid (5.0 g), isopropanol (50 ml) were added to 250 ml four necked round bottom flask, fitted with a mechanical stirrer and reflux condenser. The reaction mixture was slowly heated to 55-60° C. for complete dissolution of the mass. Tromethamine (1.36 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum-dried to afford the pure tromethamine salt of 3-[4-[2-(1,5-dimethyl-7-oxo-3-propyl-6,7-dihydro-1H-pyrazolo[4,3-d]pyrimidin-6-yl)ethoxy]phenyl]-2-ethoxypropionic acid as off white hygroscopic solid (weighs about 5.1 g, yield: 80%, purity 99% by HPLC).

IR (KBr) cm⁻¹: 3300-3250 (O—H stretch), 3120 (C—H aromatic), 2950 (—C—H aliphatic), 1689 (—COO/—CONH stretch), 1574 (—CONH, amide-II band).

¹H NMR (200 MHz, DMSO-d₆) δ: 7.13 (d, J=8.3 Hz, 2H), 6.78 (d, J=8.3 Hz, 2H), 4.48 (t, J=4.6 Hz, 2H), 4.35-4.15 (m, 5H), 4.10-3.97 (m, 1H),4.00 (s, 6H), 3.56-3.38 (m, 2H), 3.12-2.90 (m, 2H), 2.83 (t, J=7.8 Hz, 2H), 2.76 (s, 3H), 1.89-1.71 (m, 2H), 1.16 (t, J=7.0 Hz, 3H), 0.98 (t, J=7.3 Hz, 3H).

Mass m/z: 443 (M⁺+1).

### EXAMPLE 26

### 3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid dicyclohexylamine salt

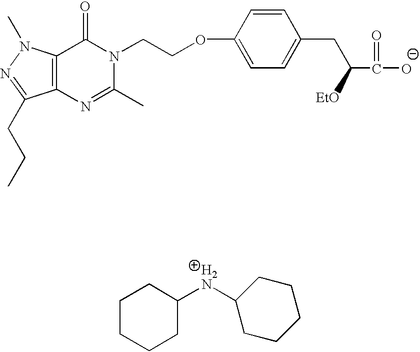

(−)-3-[4-[2-(1,5-Dimethyl)]-7-oxo-3-propyl-6,7-dihydro-1H-pyrazolo[4,3-d]pyrimidin-6-yl)ethoxy]phenyl]-2-ethoxypropionic acid (5.0 g), isopropanol (50 ml) were added to 250 ml four necked round bottom flask, fitted with a mechanical stirrer and reflux condenser. The reaction mixture was slowly heated to 55-60° C. for complete dissolution of the mass. Dicyclohexylamine (2.04 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum dried to afford the pure dicyclohexylamine salt of 3-[4-[2-(1,5-dimethyl-7-oxo-3-propyl-6,7-dihydro-1H-pyrazolo[4,3-d]pyrimidin-6-yl)ethoxy]phenyl]-2-ethoxypropionic acid as off white crystalline solid (weighs about 5.6 g, yield: 80%, mp: 132-134° C., purity 99% by HPLC).

IR (KBr) cm⁻¹: 3150 (C—H aromatic), 2950 (—C—H aliphatic), 1690 (—COO/—CONH stretch), 1574 (—CONH amide-II band).

¹H NMR (200 MHz, DMSO-d₆) δ: 7.13 (d, J=8.3 Hz, 2H), 6.78 (d, J=8.3 Hz, 2H), 4.48 (t, J=4.6 Hz, 2H), 4.35-4.15 (m, 5H), 4.10-3.97 (m, 1H), 3.56-3.38 (m, 2H), 3.12-2.90 (m, 2H), 2.83 (t, J=7.8 Hz, 2H), 2.76 (s, 3H), 2.60-1.00 (m, 22H), 1.89-1.71 (m, 2H), 1.16 (t, J=7.0 Hz, 3H), 0.98 (t, J=7.3 Hz, 3H).

Mass m/z: 443 (M⁺+1), Anal. Calcd: C₃₅H₅₃N₅O₅; % C 67.41; % H: 8.5; % N 11.23; Found % C 67.25; % H 8.4; % N 11.15.

### EXAMPLE 27

### 3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid N-octylglucamine salt

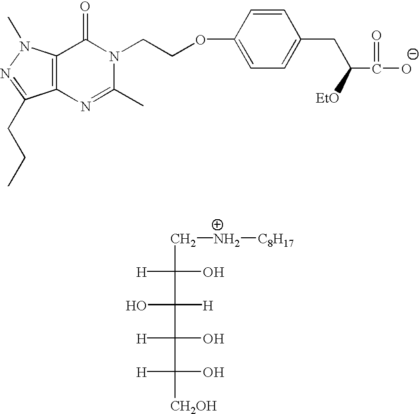

3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid (5.0 g), isopropanol (50 ml) were added to 250 ml four necked round bottom flask, fitted with a mechanical stirrer and reflux condenser. The reaction mixture was slowly heated to 55-60° C. for complete dissolution of the mass. N-octylglucamine (3.31 g) was added to the reaction mixture at 60° C. in about 10 minutes under stirring. Maintained gentle reflux of the reaction mixture for 12-14 h and monitored the progress of the reaction. The reaction mixture was cooled to RT and stirred for 2-3 h at room temperature. The precipitated product was filtered, vacuum dried to afford the pure N-octylglucamine salt of 3-[4-[2-(1,5-dimethyl-7-oxo-3-propyl-6,7-dihydro-1H-pyrazolo[4,3-d]pyrimidin-6-yl)ethoxy]phenyl]-2-ethoxypropionic acid as off white hygroscopic solid (weighs about 6.6 g, yield: 80%, purity 99% by HPLC).

IR (KBr) cm⁻¹: 3350-3300 (O—H stretch), 3150 (C—H aromatic), 2930 (—C—H aliphatic), 1689 (—COO/—CONH stretch), 1576 (—CONH amide-II band).

¹H NMR (200 MHz, DMSO-d₆) δ: 7.13 (d, J=8.3 Hz, 2H), 6.78 (d, J=8.3 Hz, 2H), 4.48 (t, J=4.6 Hz, 2H), 4.35-4.15 (m, 5H), 4.20-3.01 (m, 8H), 4.01 (m, 1H), 4.10-3.97 (m, 1H), 3.56-3.38 (m, 2H), 3.12-2.90 (m, 2H), 2.83 (t, J=7.8 Hz, 2H), 2.76 (s, 3H), 1.89-1.71 (m, 2H), 1.42-1.00 (m, 16H), 1.16 (t, J=7.0 Hz, 3H), 0.98 (t, J=7.3 Hz, 3H).

Mass m/z: 443 (M⁺+1).

### EXAMPLE 28

### 3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid N-methylglucamine salt

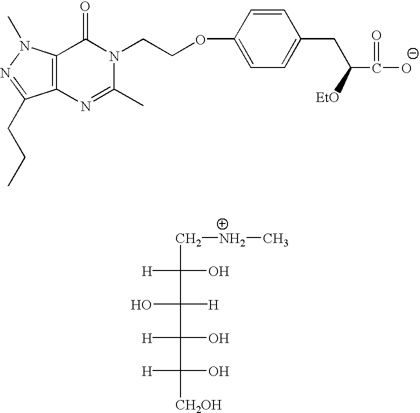

3-[4-[2-(1,5-Dimethyl-7-oxo-3-propyl-6,7-dihydro- 1H-pyrazolo[4,3-d]pyrimidin-6-yl)ethoxy]phenyl]-2-ethoxypropionic acid (5.0 g), isopropanol (50 ml) were added to 250 ml four necked round bottom flask, fitted with a mechanical stirrer and reflux condenser. The reaction mixture was slowly heated to 55-60° C. for complete dissolution of the mass. N-methylglucamine (2.2 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum-dried to afford the pure, hygroscopic N-methylglucamine salt of 3-[4-[2-(1,5-dimethyl-7-oxo-3-propyl-6,7-dihydro-1H-pyrazolo[4,3-d]pyrimidin-6-yl)ethoxy]phenyl]-2-ethoxypropionic acid as off-white crystalline solid (weighs about 6.1 g, yield: 85 %, mp 115° C., purity 99% by HPLC).

IR (KBr) cm⁻¹: 3400-3200 (—OH stretch), 3150 (C—H aromatic), 2950 (—C—H aliphatic), 1680 (—COO/—CONH stretch), 1574 (—CONH amide-II band).

¹H NMR (200 MHz, DMSO-d₆) δ: 7.13 (d, J=8.3 Hz, 2H), 6.78 (d, J=8.3 Hz, 2H), 4.48 (t, J=4.6 Hz, 2H), 4.35-4.15 (m, 5H), 4.10-3.97 (m, 1H), 4.00-3.26 (m, 8H), 3.56-3.38 (m, 2H), 3.12-2.90 (m, 2H), 2.83 (t, J=7. Hz, 2H), 2.76 (s, 3H), 2.40 (s, 3H), 1.89-1.71 (m, 2H), 1.16 (t, J=7.0 Hz, 3H), 0.98 (t, J=7.3 Hz, 3H).

Mass m/z: 443 (M⁺+1), Anal. Calcd: C₃₀H₄₇N₅O₁₀; % C 56.5; % H: 7.37; % N 10.98; Found % C 56.35; % H 7.25; % N 10.8.

### EXAMPLE 29

### 3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid metformin salt

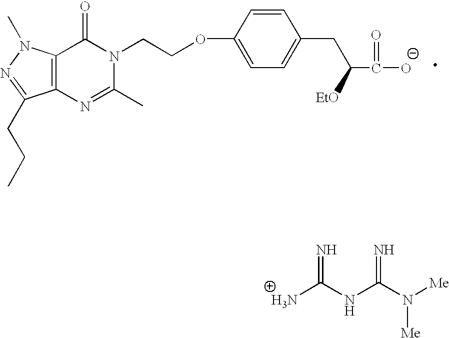

3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid (5.0 g), isopropanol (50 ml) were added to 250 ml four necked round bottom flask, fitted with a mechanical stirrer and reflux condenser. The reaction mixture was slowly heated to 55-60° C. for complete dissolution of the mass. Metformin free base (1.45 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum dried to afford the pure metformin salt of 3-[4-[2-(1,5-dimethyl-7-oxo-3-propyl-6,7-dihydro-1H-pyrazolo[4,3-d]pyrimidin-6-yl)ethoxy]phenyl]-2-ethoxypropionic acid as off white hygroscopic solid (weighs about 5.16 g, yield: 80%, purity 99% by HPLC).

IR (KBr) cm⁻¹: 3400-3300 (N—H stretch), 3190 (C—H aromatic), 2968 (—C—H aliphatic), 1687 (—COO/—CONH stretch), 1573 (—CONH amide-II band).

¹H NMR (200 MHz, DMSO-d₆) δ: 7.13 (d, J=8.3 Hz, 2H), 6.78 (d, J=8.3 Hz, 2H), 4.48 (t, J=4.6 Hz, 2H), 4.35-4.15 (m, 5H), 4.10-3.97 (m, 1H),3.56-3.38 (m, 2H), 3.12-2.90 (m, 2H), 2.90 (s, 6H), 2.83 (t, J=7.8 Hz, 2H), 2.76 (s, 3H), 1.89-1.71 (m, 2H), 1.16 (t, J=7.0 Hz, 3H), 0.98 (t, J=7.3 Hz, 3H).

Mass m/z: 443 (M⁺+1).

### EXAMPLE 30

### 3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid lysine salt

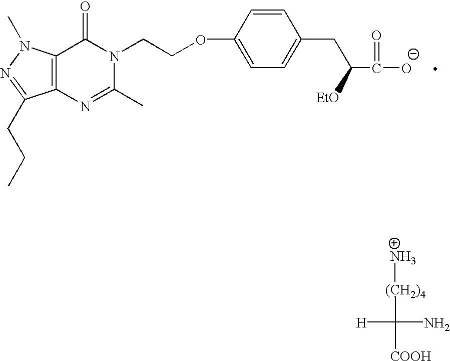

3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid (5.0 g), isopropanol (50 ml) were added to 250 ml four necked round bottom flask, fitted with a mechanical stirrer and reflux condenser. The reaction mixture was slowly heated to 55-60° C. for complete dissolution of the mass. Lysine monohydrate free base (1.65 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum dried to afford the pure lysine salt of 3-[4-[2-(1,5-dimethyl-7-oxo-3-propyl-6,7-dihydro-1H-pyrazolo[4,3-d]pyrimidin-6-yl)ethoxy]phenyl]-2-ethoxypropionic acid as off white crystalline solid (weighs about 5.98 g, yield: 90%, mp: 158-160° C., purity 99% by HPLC).

IR (KBr) cm⁻¹: 3400-3300 (N—H stretch), 3150 (C—H aromatic), 2950 (—C—H aliphatic), 1694 (—COO/—CONH stretch), 1573 (—CONH amide-II band).

¹H NMR (200 MHz, DMSO-d₆) δ: 7.13 (d, J=8.3 Hz, 2H), 6.78 (d, J=8.3 Hz, 2H), 4.48 (t, J=4.6 Hz, 2H), 4.35-4.15 (m, 5H), 4.20 (t, 1H), 4.1-3.97 (m, 1H), 3.56-3.38 (m, 2H), 3.12-2.90 (m, 2H), 3.01 (t, 2H), 2.83 (t, J=7.8 Hz, 2H), 2.76 (s, 3H), 2.20-1.5 (m, 6H), 1.89-1.71 (m, 2H), 1.16 (t, J=7.0 Hz, 3H), 0.98 (t, J=7.3 Hz, 3H).

Mass m/z: 443 (M⁺+1), Anal. Calcd: C₂₉H₄₄N₆O₇; % C 59.18; % H: 7.46; % N 14.78; Found % C 59.0; % H 7.29; % N 14.59.

(−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid was prepared according to the procedure described in our copending U.S. patent application Ser. No. 09/507,373.

[2S,N(1S)]-2-Ethoxy-3-[4-[2-(5-ethyl-1-methyl-7-oxo-3-propyl-6,7-dihydro-1H-pyrazolo[4,3-d]pyrimidin-6-y]ethoxy]phenyl]-N-(2-hydroxy-1-phenylethyl)propanamide was taken in a mixture of dioxane, water and 1M sulfuric acid at room temperature and stirred under reflux for 34 h. Water and dioxane were removed under vacuum. The residue was taken in water and extracted with ethyl acetate and the ethyl acetate layer was washed with water, dried Na₂SO₄) and evaporated to dryness to yield the crude compound. The crude compound was purified by column chromatography using 50% ethyl acetate in pet. ether as an eluent to afford (−)-3-[4-[2-(1-methyl-5-ethyl-7-oxo-3-propyl-6,7-dihydro-1H-pyrazolo[4,3-d]pyrimidin-6-yl)ethoxy]phenyl]-2-ethoxypropionic acid.

However, any other procedure for preparing (−)-3-[4-[2-(1-methyl-5-ethyl-7-oxo-3-propyl-6,7-dihydro-1H-pyrazolo[4,3-d]pyrimidin-6-yl)ethoxy]phenyl]-2-ethoxypropionic acid can be used. (±)3-[4-[2-(1-methyl-5-ethyl-7-oxo-3-propyl-6,7-dihydro-1H-pyrazolo[4,3-d]pyrimidin-6-yl)ethoxy]phenyl]-2-ethoxypropionic acid and (+)-3-[4-[2-(1-methyl-5-ethyl-7-oxo-3-propyl-6,7-dihydro-1H-pyrazolo[4,3-d]pyrimidin-6-yl)ethoxy]phenyl]-2-ethoxypropionic acid can be prepared by a similar procedure described above or any procedure for making these compounds can be used.

### EXAMPLE 31

### (−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid magnesium salt

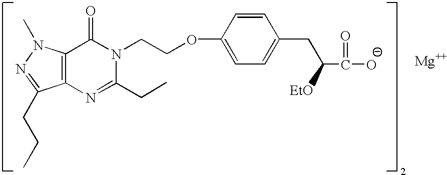

(−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid (5.0 g), methanol (50 ml) were added to 250 ml four necked round bottom flask, fitted with a mechanical stirrer and reflux condenser. The reaction mixture was slowly heated to 55-60° C. for complete dissolution of the mass and magnesium hydroxide (0.29 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum dried to afford the pure magnesium salt of (−)-3-[4-[2-(1-methyl-5-ethyl-7-oxo-3-propyl-6,7-dihydro-1H-pyrazolo[4,3-d]pyrimidin-6-yl)ethoxy]phenyl]-2-ethoxypropionic acid as off white crystalline solid, (weighs about 4.0 g, yield: 80%, purity 99% by HPLC).

IR (KBr) cm⁻¹: 3120 (C—H aromatic), 2970 (—C—H aliphatic), 1688 (—COO/—CONH stretch), 1576 (amide-II band).

¹H NMR (200 MHz, DMSO-d₆) δ: 7.13 (d, J=8.4 Hz, 2H), 6.77 (d, J=8.6 Hz, 2H), 4.50 (t, J=5.0 Hz, 2H), 4.29-4.22 (m, 5H), 4.01 (t, J=3.7 Hz, 1H), 3.69-3.35 (m, 2H), 3.10-2.94 (m, 4H), 2.84 (t, J=7.5 Hz, 2H), 1.90-1.78 (m, 2H), 1.30 (t, J=7.0 Hz, 3H), 1.16 (t, J=7.4 Hz, 3H), 0.97 (t, J=7.4 Hz, 3H).

Mass m/z: 457 (M⁺+1), Anal. Calcd: C₄₈H₆₂N₈O₁₀Mg; % C 61.67; % H: 6.63; % N 11.99; Found % C 61.58; % H 6.55; % N 11.84.

### EXAMPLE 32

### (−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid potassium salt

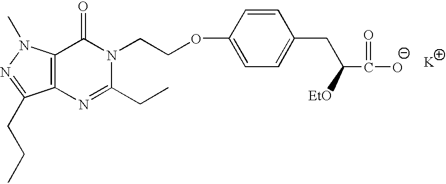

(−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid (5.0 g), isopropanol (50 ml) were added to 250 ml four necked round bottom flask, fitted with a mechanical stirrer and reflux condenser. The reaction mixture was slowly heated to 55-60° C. for complete dissolution of the mass. Potassium hydroxide (0.61 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum dried to afford the pure potassium salt of (−)-3-[4-[2-(1-methyl-5-ethyl-7-oxo-3-propyl-6,7-dihydro-1H-pyrazolo[4,3-d]pyrimidin-6-yl)ethoxy]phenyl]-2-ethoxypropionic acid as off white amorphous solid, (weighs about 4.35 g, yield: 80%, mp: 164° C., purity 99% by HPLC).

IR (KBr) cm⁻¹: 3120 (C—H aromatic), 2970 (—C—H aliphatic), 1681 (—COO/—CONH stretch), 1576 (amide-II band).

¹H NMR (200 MHz, DMSO-d₆) δ: 7.13 (d, J=8.4 Hz, 2H), 6.77 (d, J=8.6 Hz, 2H), 4.50 (t, J=5.0 Hz, 2H), 4.29-4.22 (m, 5H), 4.01 (t, J=3.7 Hz, 1H), 3.69-3.35 (m, 2H), 3.10-2.94 (m, 4H), 2.84 (t, J=7.5 Hz, 2H), 1.90-1.78 (m, 2H), 1.38 (t, J=7.3 Hz, 3H), 1.16 (t, J=7.0 Hz, 3H), 0.97 (t, J=7.4 Hz, 3H).

Mass spectrum shows m/z: 457 (M⁺+1), Anal. Calcd: C₂₄H₃₁N₄O₅K; % C 58.29; % H: 6.27; % N 11.33; Found % C 58.1; % H 6.15; % N 11.20.

### EXAMPLE 33

### (−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid calcium salt

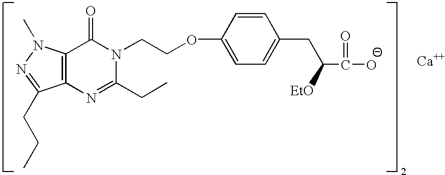

(−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid (5.0 g), methanol (50 ml) were added to 250 ml four necked round bottom flask, fitted with a mechanical stirrer and reflux condenser. The reaction mixture was slowly heated to 55-60° C. for complete dissolution of the mass. Calcium hydroxide (0.81 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum dried to afford the pure calcium salt of (−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid as off white solid, (weighs about 4.4 g, yield: 85%, mp: >260° C., purity 99% by HPLC).

IR (KBr) cm⁻¹: 3120 (C—H aromatic), 2970 (—C—H aliphatic), 1681 (—COO/—CONH stretch), 1576 (—CONH stretch).

¹H NMR (200 MHz, DMSO-d₆) δ: 7.13 (d, J=8.4 Hz, 2H), 6.77 (d, J=8.6 Hz, 2H), 4.50 (t, J=5.0 Hz, 2H), 4.29-4.22 (m, 5H), 4.01 (t, J=3.7 Hz, 1H), 3.69-3.35 (m, 2H), 3.1-2.94 (m, 4H), 2.84 (t, J=7.3 Hz, 3H), 1.90-1.78 (m, 2H), 1.38 (t, J=7.3 Hz, 3H), 1.16 (t, J=7.0 Hz, 3H), 0.97 (t, J=7.4 Hz, 3H).

Mass m/z: 457 (M⁺+1), Anal. Calcd: C₄₈H₆₂N₈O₁₀Ca; % C 60.63; % H: 6.52; % N 11.78; Found % C 60.51; % H 6.4; % N 11.60.

### EXAMPLE 34

### (−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid lithium salt

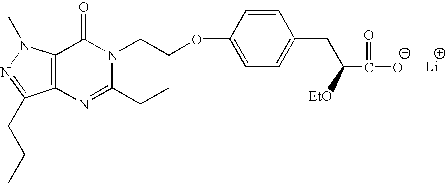

(−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid (5.0 g), methanol (50 ml) were added to 250 ml four necked round bottom flask, fitted with a mechanical stirrer and reflux condenser. The reaction mixture was slowly heated to 55-60° C. for complete dissolution of the mass. Lithium hydroxide (0.44 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum dried to afford the pure lithium salt of (−)-3-[4-[2-(1-methyl-5-ethyl-7-oxo-3-propyl-6,7-dihydro-1H-pyrazolo[4,3-d]pyrimidin-6-yl)ethoxy]phenyl]-2-ethoxypropionic acid as off white amorphous solid, (weighs about 4.55 g, yield: 90%, mp: 215-218° C., purity 99% by HPLC).

IR (KBr) cm⁻¹: 3120 (C—H aromatic), 2957 (—C—H aliphatic), 1686 (—COO/—CONH stretch), 1570 (amide-II band).

¹H NMR (200 MHz, DMSO-d₆) δ: 7.13 (d, J=8.4 Hz, 2H), 6.77 (d, J=8.6 Hz, 2H), 4.50 (t, J=5.0 Hz, 2H), 4.29-4.22 (m, 5H), 4.01 (t, J=3.7 Hz, 1H), 3.69-3.35 (m, 2H), 3.1-2.94 (m, 4H), 2.84 (t, J=7.5 Hz, 2H), 1.90-1.78 (m, 2H), 1.38 (t, J=7.3 Hz, 3H), 1.16 (t, J=7.0 Hz, 2H), 0.97 (t, J=7.4 Hz, 3H).

Mass m/z: 457 (M⁺+1), Anal. Calcd: C₂₄H₃₁N₄O₅Li; % C 62.33; % H: 6.70; % N 12.12; Found % C 62.1; % H 6.58; % N 12.10.

### EXAMPLE 35

### (−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid sodium salt

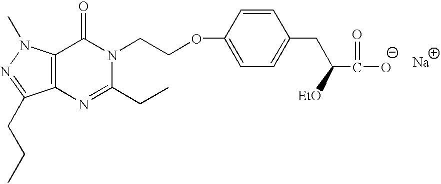

(−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid (5.0 g), methanol (50 ml) were added to 250 ml four necked round bottom flask, fitted with a mechanical stirrer and reflux condenser. The reaction mixture was slowly heated to 55-60° C. for complete dissolution of the mass. Sodium hydroxide (0.43 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum dried to afford the pure sodium salt of (−)-3-[4-[2-(1-methyl-5-ethyl-7-oxo-3-propyl-6,7-dihydro-1H-pyrazolo[4,3-d]pyrimidin-6-yl)ethoxy]phenyl]-2-ethoxypropionic acid as off white amorphous solid, (weighs about 4.25 g, yield: 80%, mp: 112-114° C., purity 99% by HPLC).

IR (KBr) cm⁻¹: 3120 (C—H aromatic), 2970 (—C—H aliphatic), 1680 (—COO/—CONH stretch), 1570 (amide-II band).

¹H NMR (200 MHz, DMSO-d₆) δ: 7.13 (d, J=8.4 Hz, 2H), 6.77 (d, J=8.6 Hz, 2H), 4.50 (t, J=5.0 Hz, 2H), 4.29-4.22 (m, 5H), 4.01 (t, J=3.7 Hz, 1H), 3.69-3.35 (m, 2H), 3.10-2.94 (m, 4H), 2.84 (t, J=7.5 Hz, 2H), 1.90-1.78 (m, 2H), 1.16 (t, J=7.0 Hz, 3H), 1.38 (t, J=7.3 Hz, 3H), 0.97 (t, J=7.4 Hz, 3H).

Mass m/z: 457 (M⁺+1), Anal. Calcd: C₂₄H₃₁N₄O₅Na; % C 60.25; % H: 6.48; % N 11.71; Found % C 60.15; % H 6.31; % N 11.58.

### EXAMPLE 36

### (−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid arginine salt

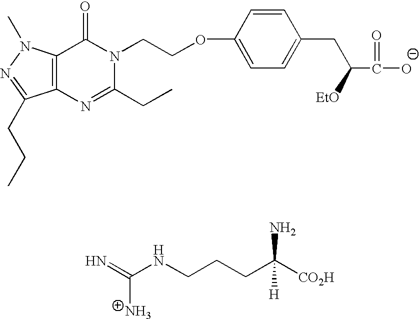

(−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid (5.0 g), isopropanol (50 ml) were added to 250 ml four necked round bottom flask, fitted with a mechanical stirrer and reflux condenser. The reaction mixture was slowly heated to 55-60° C. for complete dissolution of the mass. L-Arginine (1.9 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum dried to afford the pure arginine salt of (−)-3-[4-[2-(1-methyl-5-ethyl-7-oxo-3-propyl-6,7-dihydro-1H-pyrazolo[4,3-d]pyrimidin-6-yl)ethoxy]phenyl]-2-ethoxypropionic acid as off white amorphous solid, (weighs about 5.5 g, yield: 80%, mp: 235° C., purity 99% by HPLC).

IR (KBr) cm⁻¹: 3360 (—NH stretch), 3120 (C—H aromatic), 2957 (—C—H aliphatic), 1688 (—COO/—CONH stretch), 1573 (amide-II band).

¹H NMR (200 MHz, DMSO-d₆) δ: 7.13 (d, J=8.4 Hz, 2H), 6.77 (d, J=8.6 Hz, 2H), 4.50 (t, J=5.0 Hz, 2H), 4.29-4.22 (m, 5H), 4.01 (t, J=3.7 Hz, 1H), 3.80 (m, 2H), 3.69-3.35 (m, 2H), 3.20 (m, 3H), 3.10-2.94 (m, 4H), 2.84 (t, J=7.5 Hz, 2H), 1.90-1.78 (m, 2H), 1.60-2.00 (m, 3H), 1.38 (t, J=7.3 Hz, 3H), 1.16 (t, J=7.0 Hz, 3H), 0.97 (t, J=7.4 Hz, 3H).

Mass m/z: 457 (M⁺+1), Anal. Calcd: C₃₀H₄₆N₈O₇; % C 57.14; % H: 7.3; % N 17.75; Found % C 57; % H 7.15; % N 17.58.

### EXAMPLE 37

### (−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid R-(+) methyl benzylamine salt

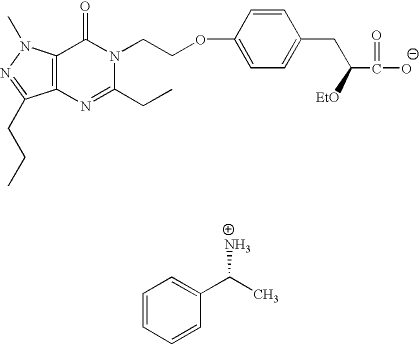

(−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid (5.0 g), isopropanol (50 ml) were added to 250 ml four necked round bottom flask, fitted with a mechanical stirrer and reflux condenser. The reaction mixture was slowly heated to 55-60° C. for complete dissolution of the mass. R-(+) methyl benzylamine (1.32 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum dried to afford the pure, hygroscopic (−)-3-[4-[2-(1-methyl-5-ethyl-7-oxo-3-propyl-6,7-dihydro-1H-pyrazolo[4,3-d]pyrimidin-6-yl)ethoxy]phenyl]-2-ethoxypropionic acid R-(+) methyl benzylamine salt as off white solid, (weighs about 4.4 g, yield: 70%, mp: 164-166° C., purity 99% by HPLC).

IR (KBr) cm⁻¹: 3120 (C—H aromatic), 2970 (—C—H aliphatic), 1689 (—COO/—CONH stretch), 1573 (amide-II band).

¹H NMR (200 MHz, DMSO-d₆) δ: 7.20-7.40 (m, 5H), 7.13 (d, J=8.4 Hz, 2H), 6.77 (d, J=8.6 Hz, 2H), 4.50 (t, J=5.0 Hz, 2H), 4.29-4.22 (m, 5H), 4.10 (m, 1H), 4.01 (t, J=3.0 Hz, 1H), 3.69-3.35 (m, 2H), 3.10-2.94 (m, 4H), 2.84 (t, J=7.5 Hz, 2H), 1.90-1.78 (m, 2H), 1.40 (d, 3H), 1.16 (t, J=7.0 Hz, 3H), 1.38 (t, J=7.3 Hz, 3H), 0.97 (t, J=7.4 Hz, 3H).

Mass m/z: 457 (M⁺+1).

### EXAMPLE-38

### (−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid S-(+)-phenylglycinol salt

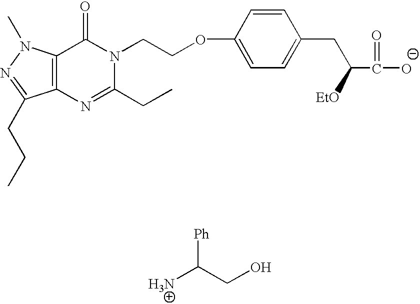

(−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid (5.0 g), isopropanol (50 ml) were added to 250 ml four necked round bottom flask, fitted with a mechanical stirrer and reflux condenser. The reaction mixture was slowly heated to 55-60° C. for complete dissolution of the mass. S-(+)-Phenylglycinol (1.5 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum dried to afford the pure S-(+)-phenylglycinol salt of (−)-3-[4-[2-(1-methyl-5-ethyl-7-oxo-3-propyl-6,7-dihydro-1H-pyrazolo[4,3-d]pyrimidin-6-yl)ethoxy]phenyl]-2-ethoxypropionic acid as off white crystalline solid, (weighs about 5.5 g, yield: 85%, mp: 88-90° C., purity 99% by HPLC).

IR (KBr) cm⁻¹: 3150 (C—H aromatic), 2950 (—C—H aliphatic), 1686 (—COO/—CONH stretch), 1570 (amide-II band).

The ¹H NMR (200 MHz, DMSO-d₆) δ: 7.40 (m, 5H), 7.13 (d, J=8.4 Hz, 2H), 6.77 (d, J=8.6 Hz, 2H), 4.50 (t, J=5.0 Hz, 2H), 4.29-4.22 (m, 5H), 4.10 (m, 1H), 4.01 (t, J=3.70 Hz, 1H), 4.00 (d, 2H), 3.60-3.80 (m, 1H), 3.69-3.35 (m, 2H), 3.10-2.94 (m, 4H), 2.84 (t, J=7.5 Hz, 2H), 1.90-1.78 (m, 2H), 1.38 (t, J=7.3 Hz, 3H), 1.16 (t, J=7.0 Hz, 3H), 0.97 (t, J=7.4 Hz, 3H).

Mass m/z: 457 (M⁺+1), Anal. Calcd: C₃₂H₄₃N₅O₆; % C 64.59; % H: 7.15; % N 11.80; Found % C 64.59; % H 7.15; % N 11.66.

### EXAMPLE 39

### (−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid aminoguanidine hydrogen carbonate salt

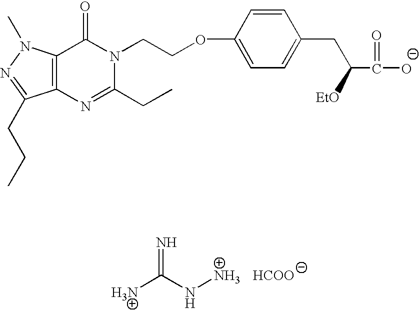

(−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid (5.0 g), isopropanol (50 ml) were added to 250 ml four necked round bottom flask, fitted with a mechanical stirrer and reflux condenser. The reaction mixture was slowly heated to 55-60° C. for complete dissolution of the mass. Aminoguanidine hydrogen carbonate (1.49 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum dried to afford the pure aminoguanidine hydrogen carbonate salt of (−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid as off white crystalline solid, (weighs about 5.2 g, yield: 80%, mp: 130° C., purity 99% by HPLC).

IR (KBr) cm⁻¹: 3450-3350 (—NH stretch), 3120 (C—H aromatic), 2970 (—C—H aliphatic), 1676 (—COO/—CONH stretch), 1573 (amide-II band).

¹H NMR (200 MHz, DMSO-d₆) δ: 7.13 (d, J=8.4 Hz, 2H), 6.77 (d, J=8.6 Hz, 2H), 4.50 (t, J=5.0 Hz, 2H), 4.29-4.22 (m, 5H), 4.01 (t, J=3.70 Hz, 1H), 3.69-3.35 (m, 2H), 3.10-2.94 (m, 4H), 2.84 (t, J=7.5 Hz, 2H), 1.90-1.78 (m, 2H), 1.38 (t, J=7.3 Hz, 3H), 1.16 (t, J=7.0 Hz, 2H), 0.97 (t, J=7.4 Hz, 3H).

Mass m/z: 457 (M⁺+1), Anal. Calcd: C₂₆H₄₀N₈O₇; % C 54.16; % H: 6.94; % N 19.44; Found % C 54.00; % H 6.78; % N 19.32.

### EXAMPLE 40

### (−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid tromethamine salt

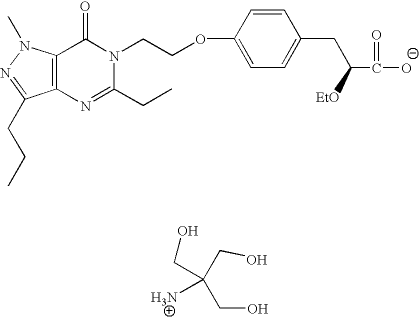

(−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid (5.0 g), isopropanol (50 ml) were added to 250 ml four necked round bottom flask, fitted with a mechanical stirrer and reflux condenser. The reaction mixture was slowly heated to 55-60° C. for complete dissolution of the mass. Tromethamine (1.32 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum dried to afford the pure tromethamine salt of (−)-3-[4-[2-(1-methyl-5-ethyl-7-oxo-3-propyl-6,7-dihydro-1H-pyrazolo[4,3-d]pyrimidin-6-yl)ethoxy]phenyl]-2-ethoxypropionic acid as off white crystalline solid, (weighs about 5.18 g, yield: 82%, mp: 116-118° C., purity 99% by HPLC).

IR (KBr) cm⁻¹: 3300-3250 (—OH stretch), 3120 (C—H aromatic), 2935 (—C—H aliphatic), 1693 (—COO/—CONH stretch), 1575 (amide-II band).

¹H NMR (200 MHz, DMSO-d₆) δ: 7.13 (d, J=8.4 Hz, 2H), 6.77 (d, J=8.1 Hz, 2H), 4.50 (t, J=5.0 Hz, 2H), 4.29-4.22 (m, 5H), 4.01 (t, J=3.70 Hz, 1H), 4.00 (s, 6H), 3.69-3.35 (m, 2H), 3.10-2.94 (m, 4H), 2.84 (t, J=7.5 Hz, 2H), 1.90-1.78 (m, 2H), 1.38 (t, J=7.3 Hz, 3H), 1.16 (t, J=7.0 Hz, 3H), 0.97 (t, J=7.4 Hz, 3H).

Mass m/z: 457 (M⁺+1), Anal. Calcd: C₂₈H₄₃N₃O₈; % C 61.20; % H: 7.83; % N 7.65; Found % C 61.05; % H 7.70; % N 7.50.

### EXAMPLE 41

### (−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid dicyclohexylamine salt

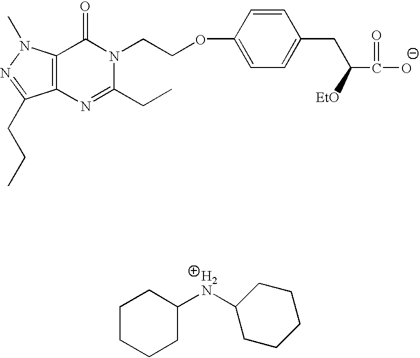

(−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid (5.0 g), isopropanol (50 ml) were added to 250 ml four necked round bottom flask, fitted with a mechanical stirrer and reflux condenser. The reaction mixture was slowly heated to 55-60° C. for complete dissolution of the mass. Dicyclohexylamine (1.98 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum dried to afford the pure dicyclohexylamine salt of (−)-3-[4-[2-(1-methyl-5-ethyl-7-oxo-3-propyl-6,7-dihydro-1H-pyrazolo[4,3-d]pyrimidin-6-yl)ethoxy]phenyl]-2-ethoxypropionic acid as off white crystalline solid, (weighs about 5.51 g, yield: 79%, mp: 138-140° C., purity 99% by HPLC.

IR (KBr) cm⁻¹: 3150 (C—H aromatic), 2933 (—C—H aliphatic), 1682 (—COO/—CONH stretch), 1571 (amide-II band).

¹H NMR (200 MHz, DMSO-d₆) δ: 7.13 (d, J=8.4 Hz, 2H), 6.77 (d, J=8.6 Hz, 2H), 4.50 (t, J=5.0 Hz, 2H), 4.29-4.22 (m, 5H), 4.01 (t, J=3.70 Hz, 1H), 3.69-3.35 (m, 2H), 3.10-2.94 (m, 4H), 2.84 (t, J=7.5 Hz, 2H), 2.60-1.00 (m, 22H), 1.90-1.78 (m, 2H), 1.38 (t, J=7.3 Hz, 3H), 1.16 (t, J=7.0 Hz, 3H), 0.97 (t, J=7.4 Hz, 3H). Mass m/z: 457 (M⁺+1), Anal. Calcd: C₃₆H₅₅N₅O₅; % C 67.81; % H: 8.63; % N 10.98; Found % C 67.65; % H 8.55; % N 10.75.

### EXAMPLE 42

### (−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid N-octylglucamine salt

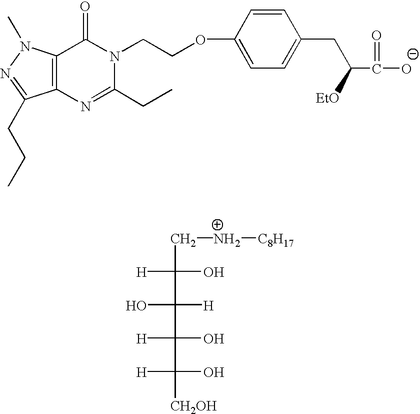

(−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid (5.0 g), isopropanol (50 ml) were added to 250 ml four necked round bottom flask, fitted with a mechanical stirrer and reflux condenser. The reaction mixture was slowly heated to 55-60° C. for complete dissolution of the mass. N-octylglucamine (3.2 g) was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum dried to afford the pure, hygroscopic N-octylglucamine salt of (−)-3-[4-[2-(1-methyl-5-ethyl-7-oxo-3-propyl-6,7-dihydro-1H-pyrazolo[4,3-d]pyrimidin-6-yl)ethoxy]phenyl]-2-ethoxypropionic acid as off white hygroscopic solid, (weighs about 6.48 g, yield: 79%, purity 99% by HPLC.

IR (KBr) cm⁻¹: 3350 (—OH stretch), 3150 (C—H aromatic), 2929 (—C—H aliphatic), 1689 (—COO/—CONH stretch), 1575 (amide-II band).

¹H NMR (200 MHz, DMSO-d₆) δ: 7.13 (d, J=8.4 Hz, 2H), 6.77 (d, J=8.6 Hz, 2H), 4.50 (t, J=5.0 Hz, 2H), 4.29-4.22 (m, 5H), 4.01 (t, J=3.70 Hz, 1H),4.20-3.06 (m, 8H), 3.69-3.35 (m, 2H), 3.10-2.94 (m, 4H), 2.84 (t, J=7.5 Hz, 2H), 1.90-1.78 (m, 2H), 1.40-1.00 (m, 16H), 1.38 (t, J=7.3 Hz, 3H), 1.16 (t, J=7.0 Hz, 3H), 0.97 (t, J=7.4 Hz, 3H).

Mass m/z: 457 (M⁺+1).

### EXAMPLE 43

### (−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid N-methylglucamine salt

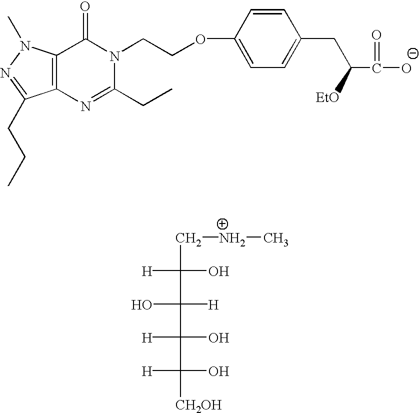

(−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid (5.0 g), isopropanol (50 ml) were added to 250 ml four necked round bottom flask, fitted with a mechanical stirrer and reflux condenser. The reaction mixture was slowly heated to 55-60° C. for complete dissolution of the mass. N-methylglucamine (2.1 g) of was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum dried to afford the pure, hygroscopic N-methylglucamine salt of (−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid as off crystalline hygroscopic solid, (weighs about 5.7 g, yield: 80%, purity 99% by HPLC).

IR (KBr) cm⁻¹: 3400-3300 (—OH stretch), 3150 (C—H aromatic), 2935 (—C—H aliphatic), 1676 (—COO/—CONH stretch), 1576 (amide-II band).

¹H NMR (200 MHz, DMSO-d₆) δ: 7.13 (d, J=8.4 Hz, 2H), 6.77 (d, J=8.6 Hz, 2H), 4.50 (t, J=5.0 Hz, 2H), 4.29-4.22 (m, 5H), 4.01 (t, J=3.70 Hz, 1H),4.00-3.26 (m, 8H), 3.69-3.35 (m, 2H), 3.10-2.94 (m, 4H), 2.84 (t, J=7.5 Hz, 2H), 2.40 (s, 3H), 1.90-1.78 (m, 2H), 1.38 (t, J=7.3 Hz, 3H), 1.16 (t, J=7.0 Hz, 3H), 0.97 (t, J=7.4 Hz, 3H).

Mass m/z: 457 (M⁺+1).

### EXAMPLE 44

### (−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid metformin salt

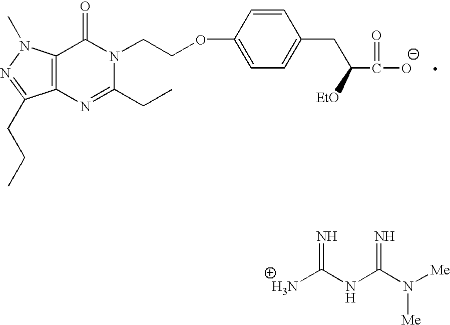

(−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid (5.0 g), isopropanol (50 ml) were added to 250 ml four necked round bottom flask, fitted with a mechanical stirrer and reflux condenser. The reaction mixture was slowly heated to 55-60° C. for complete dissolution of the mass. Metformin (1.41 g) as free base was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum dried to afford the pure, crystalline metformin salt of (−)-3-[4-[2-(1-methyl-5-ethyl-7-oxo-3-propyl-6,7-dihydro-1H-pyrazolo[4,3-d]pyrimidin-6-yl)ethoxy]phenyl]-2-ethoxypropionic acid as off white crystalline solid, (weighs about 5.45 g, yield: 85%, mp: 118° C., purity 99% by HPLC).

IR (KBr) cm⁻¹: 3350 (—OH stretch), 3150 (C—H aromatic), 2950 (—C—H aliphatic), 1677 (—COO/—CONH stretch), 1574 (amide-II band).

¹H NMR (200 MHz, DMSO-d₆) δ: 7.13 (d, J=8.4 Hz, 2H), 6.77 (d, J=8.6 Hz, 2H), 4.50 (t, J=5.0 Hz, 2H), 4.29-4.22 (m, 5H), 4.01 (t, J=3.70 Hz, 1H), 3.69-3.35 (m, 2H), 3.10-2.94 (m, 4H), 2.90 (s, 6H), 2.84 (t, J=7.5 Hz, 2H), 1.90-1.78 (m, 2H), 1.38 (t, J=7.3 Hz, 3H), 1.16 (t, J=7.0 Hz, 3H), 0.97 (t, J=7.4 Hz, 3H).

Mass m/z: 457 (M⁺+1). Anal. Calcd: C₂₈H₄₃N₉O₅; % C 57.43; % H: 7.35; % N 21.53; Found % C 57.29; % H 7.25; % N 21.40.

### EXAMPLE -45

### (−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid Lysine salt

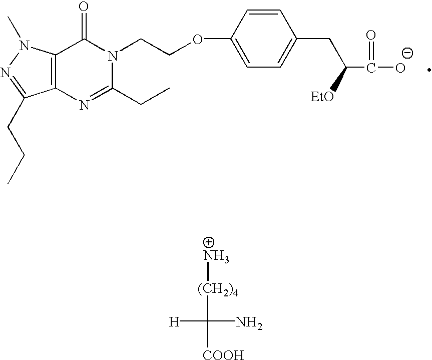

(−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid (5.0 g), isopropanol (50 ml) were added to 250 ml four necked round bottom flask, fitted with a mechanical stirrer and reflux condenser. The reaction mixture was slowly heated to 55-60° C. for complete dissolution of the mass. Lysine monohydrate (1.79 g) free base was added to the reaction mixture at 60° C. in about 10 minutes under stirring. Maintained gentle reflux of the reaction mixture for 12-14 hr and monitored the progress of the reaction. The reaction mixture was cooled to RT and stirred for 2-3 h at room temperature. The precipitated product was filtered, vacuum-dried to afford the pure lysine salt of (−)-3-[4-[2-(1-methyl-5-ethyl-7-oxo-3-propyl-6,7-dihydro-1H-pyrazolo[4,3-d]pyrimidin-6-yl)ethoxy]phenyl]-2-ethoxypropionic acid as off white amorphous solid, weighs about 5.77 g, yield: 85%, mp: 162-164° C., purity 99% by HPLC).

IR (KBr) cm⁻¹: 3120 (C—H aromatic), 2965 (—C—H aliphatic), 1691 (—COO/—CONH stretch), 1573 (amide-II band).

¹H NMR (200 MHz, DMSO-d₆) δ: 7.13 (d, J=8.4 Hz, 2H), 6.77 (d, J=8.6 Hz, 2H), 4.50 (t, J=5.0 Hz, 2H), 4.29-4.22 (m, 5H), 4.20 (t, 1H), 4.01 (t, J=3.70 Hz, 1H), 3.69-3.35 (m, 2H), 3.10 (t, 2H), 3.10-2.94 (m, 4H), 2.90 (s, 6H), 2.84 (t, J=7.5 Hz, 2H), 2.11-1.5 (m, 6H), 1.90-1.78 (m, 2H), 1.38 (t, J=7.3 Hz, 3H), 1.16 (t, J=7.0 Hz, 3H), 0.97 (t, J=7.4 Hz, 3H).

Mass m/z; 457 (M⁺+1). Anal. Calcd: C₃₀H₄₈N₆O₈; % C 58.06; % H: 7.74; % N 13.54; Found % C 57.9; % H 7.55; % N 13.38.

(−)2-Ethoxy-3-[4-[2-[2-ethyl-6-oxo-4-phenyl-1,6-dihydropyrimidin-1-yl]ethoxy]phenyl]propanoic acid was prepared according to the procedure described in our copending U.S. patent application Ser. No. 09/507,371:

A solution of [2S, N(1S)]-2-ethoxy-3-[4-[2-[2-ethyl-6-oxo-4-phenyl-1,6-dihydropyrimidin-1-yl]ethoxy]phenyl]-N-(2-hydroxy-1-phenylethyl) propanamide (295 mg, 0.53 mmol) in a mixture of 1M sulfuric acid (7.7 mL) and dioxane/water (1:1, 14 mL) was heated at 90° C. for 48 h and the pH of the mixture was adjusted to 4 by the addition of aqueous sodium bicarbonate solution. The mixture was extracted with ethyl acetate and the combined ethyl acetate layers were washed with water, brine, dried (Na₂SO₄) and evaporated to yield (−)2-ethoxy-3-[4-[2-[2-ethyl-6-oxo-4-phenyl-1,6-dihydropyrimidin-1-yl]ethoxy]phenyl]propanoic acid as a colorless solid.

However, any other procedure for preparing (−)2-ethoxy-3-[4-[2-[2-ethyl-6-oxo-4-phenyl-1,6-dihydropyrimidin-1-yl]ethoxy]phenyl]propanoic acid can be used. (±)3-[4-[2-(1-methyl-5-ethyl-7-oxo-3-propyl-6,7-dihydro-1H-pyrazolo[4,3-d]pyrimidin-6-yl) ethoxy]phenyl]-2-ethoxypropionic acid and (+)-3-[4-[2-(1-methyl-5-ethyl-7-oxo-3-propyl-6,7-dihydro-1H-pyrazolo[4,3-d]pyrimidin-6-yl)ethoxy]phenyl]-2-ethoxypropionic acid can be prepared by a similar procedure described above or any other procedure for making these compounds can be used.

### EXAMPLE 46

### (−)2-Ethoxy-3-[4-[2-[2-ethyl-6-oxo-4-phenyl-1,6-dihydropyrimidin-1-yl]ethoxy]phenyl]propanoic acid potassium salt

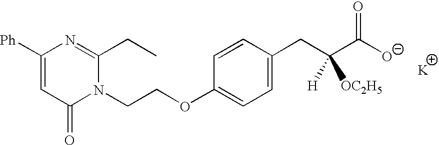

A mixture of (−)2-ethoxy-3-[4-[2-[2-ethyl-6-oxo-4-phenyl-1,6-dihydropyrimidin-1-yl]ethoxy]phenyl]propanoic acid (5.0 g) and isopropanol (75 ml) was refluxed to obtain clear solution. Aqueous potassium hydroxide solution (0.63 g in 2 ml water) was added slowly at reflux temperature under stirring and continued stirring for 2-3 h. Then the reaction mass was cooled to room temperature and continued stirring for further 12 h. The precipitated solid was filtered and dried to yield the title compound, (weights about 4.5 g, mp (DSC) 240.9° C.).

IR (KBr) cm⁻¹: 3439.8, 2976.2, 2934.3, 2877.4, 1667.1, 1602.7, 1551.4, 1509.9, 1448.7, 1407.3, 1363.2, 1241.8, 1178.2, 1108.7, 957.9, 898.3, 864.2, 815.7, 781.9, 696.6, 643.6.

p-XRD: 5.56, 7.46, 8.30, 14.36, 14.72, 15.74, 16.84, 17.06, 17.88, 18.40, 18.68, 19.44, 24.30, 24.42, 24.92 (2θ).

### EXAMPLE 47

### (−)2-Ethoxy-3-[4-[2-[2-ethyl-6-oxo-4-phenyl-1,6-dihydropyrimidin-1-yl]ethoxy]phenyl]propanoic acid sodium salt

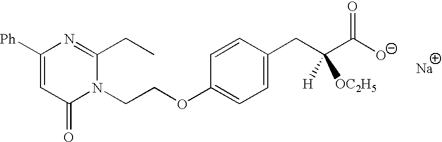

A mixture of (−)2-ethoxy-3-[4-[2-[2-ethyl-6-oxo-4-phenyl-1,6-dihydropyrimidin-1-yl]ethoxy]phenyl]propanoic acid (5.0 g) and isopropanol (75 ml) was refluxed to obtain clear solution. Sodium methoxide (0.615 g) was added at reflux temperature under stirring and continued stirring for 5-6 h. Then the reaction mass was cooled to room temperature, filtered and dried to yield the title compound (weights about 4.6 g, mp (DSC) 251.19° C.).

IR (KBr) cm⁻¹: 3437.2, 2977.7, 2879.1, 1667.7, 1605.4, 1552.5, 1509.7, 1449.0,, 1407.6, 1362.9, 1282.8, 1242.0, 1178.2, 1108.9, 1047.4, 958.6, 898.9, 863.4, 815.4, 781.3, 737.5, 696.1, 644.6, 519.0.

p-XRD: 5.52, 7.38, 8.24, 14.42, 14.68, 17.0, 17.88, 19.46, 19.80, 23.38, 24.26, 24.44, 26.40 (2θ).

### EXAMPLE 48

### (−)2-Ethoxy-3-[4-[2-[2-ethyl-6-oxo-4-phenyl-1,6-dihydropyrimidin-1-yl]ethoxy]phenyl]propanoic acid L-arginine salt

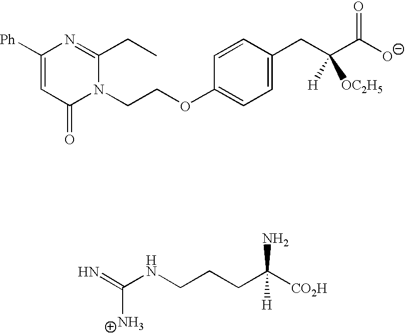

A mixture of (−)2-ethoxy-3-[4-[2-[2-ethyl-6-oxo-4-phenyl-1,6-dihydropyrimidin-1-yl]ethoxy]phenyl]propanoic acid (10 g) and isopropanol (200 ml) was refluxed to obtain clear solution. Aqueous L arginine solution (3.99 g in 6 ml water) was added slowly at reflux temperature under stirring and continued stirring for 2-3 h. Then the reaction mass was cooled to room temperature and continued stirring for further 12 h. The precipitated solid was filtered and dried to yield the title compound (weights about 8.5 g, mp (DSC) 187° C.).

IR (KBr) cm⁻¹: 3358.9, 3064.4, 2974.3, 1665.5, 1591.0, 1544.9, 1511.2, 1448.9, 1406.3, 1323.7, 1240.1, 1178.9, 1111.2, 1046.2, 956.8, 896.7, 780.5, 697.3, 542.0.

p-XRD: 3.68, 7.34, 11.34, 12.74, 14.18, 14.86, 15.12, 16.64, 19.48, 20.0, 20.54, 20.86, 22.70, 30.02 (2θ).

### EXAMPLE 49

### (−)2-Ethoxy-3-[4-[2-[2-ethyl-6-oxo-4-phenyl-1,6-dihydropyrimidin-1-yl]ethoxy]phenyl]propanoic acid L-lysine salt

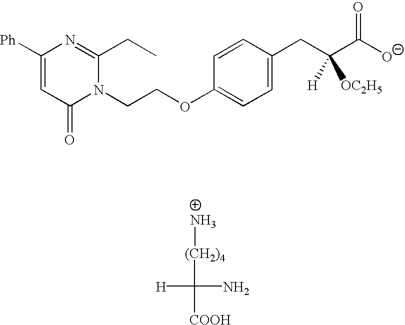

A mixture of (−)2-ethoxy-3-[4-[2-[2-ethyl-6-oxo-4-phenyl-1,6-dihydropyrimidin-1-yl]ethoxy]phenyl]propanoic acid (5.0 g) and isopropanol (75 ml) was refluxed to obtain clear solution. Aqueous L lysine solution (1.88 g in 3 ml water) was added slowly at reflux temperature under stirring and continued stirring for 12-15 h. Then the reaction mass was cooled to room temperature and the precipitated solid was filtered and dried to yield the title compound (weights about 4.3 g, mp (DSC) 114.93° C.).

IR (KBr) cm⁻¹: 3413.9, 2934.8, 1663.6, 1571.6, 15457, 1511.8, 1449.0, 1405.5, 1323.2, 1241.8, 1179.3, 1112.1, 1046.5, 956.0,898.9, 853.9, 782.5, 736.6, 698.9, 644.7, 557.8.

p-XRD: 4.42, 7.58, 7.84, 7.98, 11.64, 11.96, 13.62, 15.76, 16.14, 16.36, 16.54, 17.84, 19.32, 19.46 (2θ).

### EXAMPLE 50

### (−)2-Ethoxy-3-[4-[2-[2-ethyl-6-oxo-4-phenyl-1,6-dihydropyrimidin-1-yl]ethoxy]phenyl]propanoic acid t-butyl amine salt

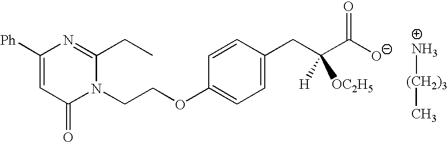

A mixture of (−)2-ethoxy-3-[4-[2-[2-ethyl-6-oxo-4-phenyl-1,6-dihydropyrimidin-1-yl]ethoxy]phenyl]propanoic acid (2.0 g) and isopropanol (15 ml) was heated to reflux to get the clear solution. T-Butylamine (0.35 g) was added slowly dropwise and continued reflux for 2-3 hrs. Then the reaction mass was cooled to room temperature and continued stirring for further 2 h. Then slowly reaction mass cooled to 15° C., continued stirring for overnight. Filtered the compound at 10-15° C., washed with isopropanol (5 ml) dried under high vacuum at 50-60° C. over a period of 8-10 hr to yield the title compound (weights about 2.0 g).

DSC: 90.89-105.46 (Endo), 118.30 (Exo), 158.37 (Endo)

p-XRD: 4.98, 6.84, 8.52, 10.14, 13.34, 14.64, 15.76, 17.32, 18.16, 19.54, 20.62, 22.20, 23.10, 24.76, 25.60, 27.36, 31.54, 32.46 Cm-¹.

IR: 3428, 2977, 1676, 1545, 1512, 1470, 1448, 1402, 1319, 1238, 1113, 1047, 900, 854, 781, 699 Cm-¹.

The compounds of the present invention lowered random blood sugar level, triglyceride, total cholesterol, LDL, VLDL and increased HDL. This was demonstrated by in vitro as well as in vivo animal experiments.

Demonstration of Efficacy of Compounds

A) In vitro

a) Determination of hPPARα Activity

Ligand binding domain of hPPARα was fused to DNA binding domain of Yeast transcription factor GAL4 in eucaryotic expression vector. Using superfect (Qiagen, Germany) as transfecting reagent HEK-293 cells were transfected with this plasmid and a reporter plasmid harboring the luciferase gene driven by a GAL4 specific promoter. Compound was added at different concentrations after 42 hrs of transfection and incubated overnight. Luciferase activity as a function of compound binding/activation capacity of PPARα was measured using Packard Luclite kit (Packard, USA) in Top Count (Ivan Sadowski, Brendan Bell, Peter Broag and Melvyn Hollis. Gene. 1992. 118: 137-141; Superfect Transfection Reagent Handbook. February 1997. Qiagen, Germany).

b) Determination of hPPARγ Activity

Ligand binding domain of hPPARγ1 was fused to DNA binding domain of Yeast transcription factor GAL4 in eucaryotic expression vector. Using lipofectamine (Gibco BRL, USA) as transfecting reagent HEK-293 cells were transfected with this plasmid and a reporter plasmid harboring the luciferase gene driven by a GAL4 specific promoter. Compound was added at 1 μM concentration after 48 hrs of transfection and incubated overnight. Luciferase activity as a function of drug binding/activation capacity of PPARγ1 was measured using Packard Luclite kit (Packard, USA) in Packard Top Count (Ivan Sadowski, Brendan Bell, Peter Broag and Melvyn Hollis. Gene. 1992. 118: 137-141; Guide to Eukaryotic Transfections with Cationic Lipid Reagents. Life Technologies, GIBCO BRL, USA).

| Example No. | Concentration | PPARα | PPARγ | Concentration |
|---|---|---|---|---|
| 11 | 50 μM | 13.8 | 1 μM | 27.0 |
| 16 | 50 μM | 3.4 | 1 μM | 13.5 |
| 20 | 50 μM | 3.5 | 1 μM | 13.0 |
| 23 | 50 μM | 3.4 | 1 μM | 11.5 |
| 36 | 50 μM | 3.3 | 1 μM | 11.4 |
| 38 | 50 μM | 3.3 | 1 μM | 12.0 |
| 41 | 50 μM | 3.1 | 1 μM | 13.0 |
| 44 | 50 μM | 2.5 | 1 μM | 12.0 |

c) Determination of HMG CoA Reductase Inhibition Activity

Liver microsome bound reductase is prepared from 2% cholestyramine fed rats at mid-dark cycle. Spectrophotometric assays are carried out in 100 mM KH₂PO₄, 4 mM DTT, 0.2 mM NADPH0.3 mM HMG CoA and 125 μg of liver microsomal enzyme. Total reaction mixture volume is kept as 1 ml. Reaction is started by addition of HMG CoA. Reaction mixture is incubated at 37° C. for 30 min and decrease in absorbance at 340 nm is recorded. Reaction mixture without substrate is used as blank (Goldstein, J. L and Brown, M. S. Progress in understanding the LDL receptor and HMG CoA reductase, two membrain proteins that regulate the plasma cholesterol. J. Lipid Res. 1984, 25: 1450-1461). The test compounds will inhibit the HMG CoA reductase enzyme.

In vivo

a) Efficacy in Genetic Models

Mutation in colonies of laboratory animals and different sensitivities to dietary regimens have made the development of animal models with non-insulin dependent diabetes and hyperlipidemia associated with obesity and insulin resistance possible. Genetic models such as db/db and ob/ob (Diabetes, (1982) 31(1): 1-6) mice and zucker fa/fa rats have been developed by the various laboratories for understanding the pathophysiology of disease and testing the efficacy of new antidiabetic compounds (Diabetes, (1983) 32: 830-838; Annu. Rep. Sankyo Res. Lab. (1994). 46: 1-57). The homozygous animals, C57 BL/KsJ-db/db mice developed by Jackson Laboratory, US, are obese, hyperglycemic, hyperinsulinemic and insulin resistant (J. Clin. Invest., (1990) 85:962-967), whereas heterozygous are lean and normoglycemic. In db/db model, mouse progressively develops insulinopenia with age, a feature commonly observed in late stages of human type II diabetes when blood sugar levels are insufficiently controlled. The state of pancreas and its course vary according to the models. Since this model resembles that of type II diabetes mellitus, the compounds of the present invention were tested for blood sugar and triglycerides lowering activities.

Male C57BL/KsJ-db/db mice of 8 to 14 weeks age, having body weight range of 35 to 60 grams, bred at Dr. Reddy's Research Foundation (DRF) animal house, were used in the experiment. The mice were provided with standard feed (National Institute of Nutrition (NIN), Hyderabad, India) and acidified water, ad libitum. The animals having more than 350 mg/dl blood sugar were used for testing. The number of animals in each group was 4.

Test compounds were suspended on 0.25% carboxymethyl cellulose and administered to test group at a dose of 0.1 mg to 30 mg/kg through oral gavage daily for 6 days. The control group received vehicle (dose 10 ml/kg). On 6th day the blood samples were collected one hour after administration of test compounds/vehicle for assessing the biological activity.

The random blood sugar and triglyceride levels were measured by collecting blood (100 μl) through orbital sinus, using heparinised capillary in tubes containing EDTA which was centrifuged to obtain plasma. The plasma glucose and triglyceride levels were measured spectrometrically, by glucose oxidase and glycerol-3-PO₄ oxidase/peroxidase enzyme (Dr. Reddy's Lab. Diagnostic Division Kits, Hyderabad, India) methods respectively.

The blood sugar and triglycerides lowering activities of the test compound was calculated according to the formula.

No adverse effects were observed for any of the mentioned compounds of invention in the above test.

| Example No. | Dose (mg/kg) | Reduction in Blood Glucose Level (%) |
|---|---|---|
| Example 11 | 0.1 | 61 |

The ob/ob mice are obtained at 5 weeks of age from Bomholtgard, Denmark and are used at 8 weeks of age. Zucker fa/fa fatty rats are obtained from IffaCredo, France at 10 weeks of age and are used at 13 weeks of age. The animals are maintained under 12 hour light and dark cycle at 25+1° C. Animals are given standard laboratory chow (NIN Hyderabad, India) and water, ad libitum (Fujiwara, T., Yoshioka, S., Yoshioka, T., Ushiyama, I and Horikoshi, H. Characterization of new oral antidiabetic agent CS-045. Studies in KK and ob/ob mice and Zucker fatty rats. Diabetes. 1988. 37: 1549-1558).

The test compounds will be administered at 0.1 to 30 mg/kg/day dose for 9 days. The control animals receives the vehicle (0.25% carboxymethylcellulose, dose 10 ml/kg) through oral gavage.

The blood samples can be collected in fed state 1 hour after drug administration on 0 and 9 day of treatment. The blood can be collected from the retro-orbital sinus through heparinised capillary in EDTA containing tubes. After centrifugation, plasma sample will be separated for triglyceride, glucose, free fatty acid, total cholesterol and insulin estimations. Measurement of plasma triglyceride, glucose, total cholesterol can be done using commercial kits (Dr. Reddy's Laboratory, Diagnostic Division, India). The plasma free fatty acid will be measured using a commercial kit from Boehringer Mannheim, Germany. The plasma insulin can be measured using a RIA kit (BARC India). The reduction of various parameters examined will be calculated according to the formula given below.

In ob/ob mice oral glucose tolerance test is performed after 9 days treatment. Mice are fasted for 5 hrs and challenged with 3 gm/kg of glucose orally. The blood samples are collected at 0, 15, 30, 60 and 120 min for estimation of plasma glucose levels.

b) Plasma Triglyceride and Cholesterol Lowering Activity in Hypercholesterolemic Rat Models

Male Sprague Dawley rats (NIN stock) are bred in DRF animal house. Animals are maintained under 12 hour light and dark cycle at 25±1° C. Rats of 180-200 gram body weight range were used for the experiment. Animals are made hypercholesterolemic by feeding 2% cholesterol and 1% sodium cholate mixed with standard laboratory chow [National Institute of Nutrition (NIN), Hyderabad, India] for 6 days. Throughout the experimental period the animals are maintained on the same diet (Petit, D., Bonnefis, M. T., Rey, C and Infante, R. Effects of ciprofibrate on liver lipids and lipoprotein synthesis in normo- and hyperlipidemic rats. Atherosclerosis. 1988. 74: 215-225).

The test compounds can be administered orally at a dose 0.1 to 30 mg/kg/day for 3 days. Control group is treated with vehicle alone (0.25% Carboxymethylcellulose; dose 10 ml/kg).

The blood samples can be collected in fed state 1 hour after drug administration on 0 and 3 day of compound treatment. The blood can be collected from the retro-orbital sinus through heparinised capillary in EDTA containing tubes. After centrifugation, plasma sample will be separated for total cholesterol, HDL and triglyceride estimations. Measurement of plasma triglyceride, total cholesterol and HDL are done using commercial kits (Dr. Reddy's Laboratory, Diagnostic Division, India). LDL and VLDL cholesterol can be calculated from the data obtained for total cholesterol, HDL and triglyceride. The reduction of various parameters examined are calculated according to the formula given below.

c) Plasma Triglyceride and Total Cholesterol Lowering Activity in Swiss Albino Mice and Guinea Pigs

Male Swiss albino mice (SAM) and male Guinea pigs were obtained from NIN and housed in DRF animal house. All these animals were maintained under 12 hour light and dark cycle at 25±1° C. Animals were given standard laboratory chow (NIN Hyderabad, India) and water, ad libitum. SAM of 20-25 g body weight range and Guinea pigs of 500-700 g body weight range were used (Oliver, P., Plancke, M. O., Marzin, D., Clavey, V., Sauzieres, J and Fruchart, J. C. Effects of fenofibrate, gemfibrozil and nicotinic acid on plasma lipoprotein levels in normal and hyperlipidemic mice. Atherosclerosis. 1988. 70: 107-114).

The test compounds were administered orally to Swiss albino mice at 0.3 to 30 mg/kg/day dose for 6 days. Control mice were treated with vehicle (0.25% Carboxymethylcellulose; dose 10 ml/kg). The test compounds were administered orally to Guinea pigs at 0.3 to 30 mg/kg/day dose for 6 days. Control animals were treated with vehicle (0.25% Carboxymethylcellulose; dose 5 ml/kg).

The blood samples were collected in fed state 1 hour after drug administration on 0 and 6 day of treatment. The blood was collected from the retro-orbital sinus through heparinised capillary in EDTA containing tubes. After centrifugation, plasma sample was separated for triglyceride and total cholesterol (Wieland, O. Methods of Enzymatic analysis. Bergermeyer, H. O., Ed., 1963. 211-214; Trinder, P. Ann. Clin. Biochem. 1969. 6: 24-27). Measurement of plasma triglyceride, total cholesterol and HDL were done using commercial kits (Dr. Reddy's Diagnostic Division, Hyderabad, India).

| Example No. | Dose (mg/kg) | Triglyceride Lowering (%) |
|---|---|---|
| 1 | 3 | 87 |
| 3 | 3 | 81 |
| 11 | 0.3 | 58 |
| 12 | 0.3 | 37 |
| 31 | 3 | 84 |
| 44 | 3 | 80 |
| 45 | 3 | 81 |

d) Body Weight Reducing Effect in Cholesterol Fed Hamsters

Male Syrian Hamsters are procured from NIN Hyderabad, India. Animals are housed at DRF animal house under 12 hour light and dark cycle at 25±1° C. with free access to food and water. Animals are maintained with 1% cholesterol containing standard laboratory chow (NIN) from the day of treatment.

The test compounds can be administered orally at 1 to 30 mg/kg/day dose for 15 days. Control group animals are treated with vehicle (Mill Q water, dose 10 ml/kg/day). Body weights are measured on every 3^{rd} day.

Formulae for calculation:

1. Percent reduction in Blood sugar/triglycerides/total cholesterol were calculated according to the formula:

$$Percent reduction ( \% ) = [ 1 - \frac{TT / OT}{TC / OC} ] \times 100$$

OC=Zero day control group value

OT=Zero day treated group value

TC=Test day control group value

TT=Test day treated group value

2. LDL and VLDL cholesterol levels were calculated according to the formula:

$$LDL cholesterol in mg / d 1 = [ Total cholesterol - HDL cholesterol - \frac{Triglyceride}{5} ] mg / d 1$$

VLDL cholesterol in mg/dl=[Total cholesterol−HDL cholesterol−LDL cholesterol] mg/dl.

Single Dose Oral Pharmacokinetic Studies

Male Wistar rats (220-250 gm) were used in the experiments. The animals were maintained under standard laboratory conditions and had free access to feed and water ad libitum. Before experimentation animals were fasted overnight (˜15 h) during which they had free access to water ad libitum.

An amount equivalent to 30 mg of drug was weighed accurately and transferred into a clean mortar and triturated to obtain a fine powder. To this 0.5 ml of 0.25% sodium carboxy methyl cellulose (sodium CMC) was added to obtain a paste. To the obtained paste remaining 2.5 ml of sodium CMC was added to make up the volume to 3 ml. Based on the animal weight appropriate volume (body weight×3) of the prepared suspension was administered through oral gavage.

After dosing, at designated time points (0.5, 1, 2, 3, 5, 8, 12 and 24 h) 200 μl of blood was collected from retro orbital plexus into 0.5 ml eppendorff tubes containing EDTA (10 μl of 200 mg/ml solution in Milli Q water). Blood was centrifuged at 12,800 rpm for 5 min and obtained plasma and stored at −20° C. till further analysis.

100μl plasma was transferred into a clean and dry centrifuge tube. To this internal standard (10 μl of 100 μg/ml) was added and extracted with 2 ml of extraction recovery solvent. The contents were vortexed for 2 min, followed by centrifugation for 10 min at 2800 rpm. Clear organic layer (2×0.75 ml) was separated and dried under nitrogen gas at 50° C. The residue was reconstituted with 150 μl of mobile phase and vortexed for 20 sec, from this 50 μl was injected onto HPLC column.

Pharmacokinetic parameters were calculated by non-compartmental model analysis. The peak plasma concentration (Cₘₐₓ) and the corresponding time (Tₘₐₓ)were directly obtained from the raw data. The area under the plasma concentration versus time curve up to the last quantifiable time point, AUC₍₀₋ₜ₎ was obtained by the linear and log-linear trapezoidal summation. The AUC₍₀₋ₜ₎ extrapolated to infinity (i.e., AUC_{(0-∞)}) by adding the quotient of Cₗₐₛₜ/Kₑₗ, where Cₗₐₛₜ represents the last measurable time concentration and Kₑₗ represents the apparent terminal rate constant. Kₑₗ was calculated by the linear regression of the log-transformed concentrations of the drug in the terminal phase. The half-life of the terminal elimination phase was obtained using the relationship t_{½}=0.693/Kₑₗ.

| Example | AUC _{(0-∞)} | AUC ₍₀₋ₜ₎ | Cₘₐₓ | | | |
|---|---|---|---|---|---|---|
| No. | (μg · hr/ml | (μg · hr/ml | (μg/ml) | Tₘₐₓ (h) | Kₑₗ (h⁻¹) | t_{½}(h) |
| 4 | 38.36 ± | 40.55 ± | 38.67 ± | 0.50 ± | 0.31 ± | 1.97 ± |
| | 6.76 | 6.93 | 10.76 | 0.00 | 0.15 | 0.46 |
| 7 | 51.43 ± | 51.65 ± | 40.25 ± | 0.90 ± | 0.38 ± | 1.91 ± |
| | 16.24 | 16.34 | 16.93 | 0.22 | 0.09 | 0.41 |
| 10 | 74.07 ± | 75.89 ± | 54.21 ± | 0.50 ± | 0.34 ± | 2.16 ± |
| | 21.26 | 21.03 | 19.66 | 0.00 | 0.08 | 0.58 |
| 31 | 65.93 ± | 64.70 ± | 19.15 ± | 2.75 ± | 0.34 ± | 2.24 ± |
| | 15.54 | 15.82 | 5.01 | 1.50 | 0.12 | 0.87 |
| 40 | 78.55 ± | 77.80 ± | 34.19 ± | 0.75 ± | 0.60 ± | 1.20 ± |
| | 19.49 | 19.27 | 10.03 | 0.29 | 0.13 | 0.25 |
| 45 | 101.29 ± | 99.33 ± | 24.22 ± | 2.38 ± | 0.42 ± | 1.72 ± |
| | 14.76 | 15.42 | 9.94 | 2.06 | 0.09 | 0.38 |
| 47 | 83.28 ± | 82.25 ± | 39.54 ± | 0.50 ± | 0.34 ± | 2.07 ± |
| | 10.45 | 10.61 | 10.43 | 0.00 | 0.04 | 0.22 |
| 46 | 62.91 ± | 61.25 ± | 17.69 ± | 1.33 ± | 0.32 ± | 2.19 ± |
| | 8.78 | 8.86 | 5.74 | 0.75 | 0.03 | 0.20 |
| 48 | 92.57 ± | 90.12 ± | 50.44 ± | 0.50 ± | 0.29 ± | 2.18 ± |
| | 27.28 | 28.59 | 11.12 | 0.00 | 0.11 | 0.61 |

1. A pharmaceutically acceptable salt of formula (I) 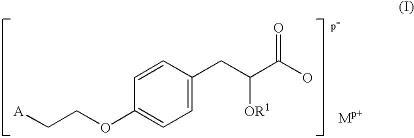
its derivatives, its analogs, its tautomeric forms, its stereoisomers, its polymorphs, or its solvates wherein R¹ represents hydrogen, alkyl or aryl group; M represents a counter ion or a moiety which forms a pharmaceutically acceptable salt; p is an integer ranging from 1 to 2; A represents a cyclic structure given below: 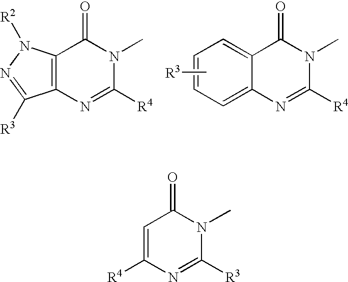
wherein R² and R³ are the same or different and independently represent hydrogen, halogen, hydroxy, nitro, cyano, alkyl or alkoxy group; R⁴ represents hydrogen, halogen, hydroxy, nitro, cyano, azido, formyl or unsubstituted or substituted groups selected from alkyl, cycloalkyl, alkoxy, aryl, heterocyclyl, heteroaryl, amino, monoalkylamino, dialkylamino or alkoxyalkyl groups.

2. A compound according to claim 1, wherein M represents a counter ion or a moiety selected from sodium, Mg, calcium, potassium, Li, glucamine, N-methyl glucamine, N-octyl glucamine, dicyclohexylamine, t-butyl amine, methyl benzylamine, tris(hydroxymethyl)amino methane (tromethamine), phenyl glycinol, lysine, arginine, metformin, aminoguanidine, aminoguanidine hydrogen carbonate, imidazole, piperazine, dimethyl piperazine, pyrrolidine, benzylamine, phenyl glycine methyl ester, phenylalanine benzyl ester or morpholine.

3. A process for the preparation of a pharmaceutically acceptable salt of the formula (I) its derivatives, its analogs, its tautomeric forms, or its stereoisomers 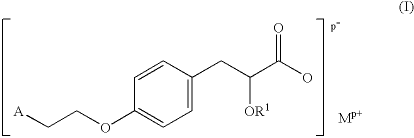
which comprises: reacting a compound of the formula (III) 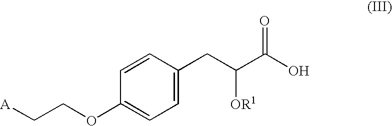
wherein R¹ represents hydrogen, alkyl or aryl group; A represents a cyclic structure given below: 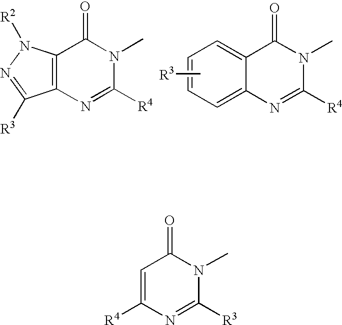
where R² and R³ are the same or different and represent hydrogen, halogen, hydroxy, nitro, cyano, alkyl, or alkoxy group; R⁴ represents hydrogen, halogen, hydroxy, nitro, cyano, azido, formyl or unsubstituted or substituted groups selected from alkyl, cycloalkyl, alkoxy, aryl, heterocyclyl, heteroaryl, amino, monoalkylamino, dialkylamino or alkoxyalkyl group, with a stoichiometric amount of a base in the presence of a solvent.

4. The process as claimed in claim 3, wherein the base used is selected from sodium hydroxide, sodium methoxide, potassium hydroxide, calcium hydroxide, lithium hydroxide, magnesium hydroxide, glucamine, N-methylglucamine, N-octylglucamine, dicyclohexylamine, t-butylamine, methyl benzylamine, tris(hydroxymethyl)aminomethane, phenyl glycinol, lysine, arginine, metformin, aminoguanidine, aminoguanidine hydrogen carbonate, imidazole, piperazine, dimethyl piperazine, pyrrolidine, benzylamine, phenyl glycine methyl ester, phenylalanine benzyl ester or morpholine.

5. The process as claimed in claim 3, wherein the reaction is effected in the presence of solvent selected from alcohols, ketones, ethers, DMF, DMSO, xylene, toluene, ethyl acetate or a mixture thereof.

6. The process as claimed in claim 4, wherein the reaction is effected in the presence of solvent selected from alcohols, ketones, ethers, DMF, DMSO, xylene, toluene, ethyl acetate or a mixture thereof.

7. The process as claimed in claim 3, wherein the reaction is carried out at a temperature in the range of −10° C. to the boiling point of the solvent employed for a period in the range of 10 minutes to 30 hours.

8. The process as claimed in claim 4, wherein the reaction is carried out at a temperature in the range of −10° C. to the boiling point of the solvent employed for a period in the range of 10 minutes to 30 hours.

9. The process as claimed in claim 5, wherein the reaction is carried out at a temperature in the range of −10° C. to the boiling point of the solvent employed for a period in the range of 10 minutes to 30 hours.

10. The process as claimed in claim 6, wherein the reaction is carried out at a temperature in the range of −10° C. to the boiling point of the solvent employed for a period in the range of 10 minutes to 30 hours.

11. A pharmaceutically acceptable salt according to claim 1, which is selected from:
(±)3-[4-[2-(2-Ethyl-4-oxo-3,4-dihydroquinazolin-3-yl)ethoxy]phenyl]-2-ethoxypropanoic acid phenyl glycinol salt;
(+)3-[4-[2-(2-Ethyl-4-oxo-3,4-dihydroquinazolin-3-yl)ethoxy]phenyl]-2-ethoxypropanoic acid phenyl glycinol salt;
(−)3-[4-[2-(2-Ethyl-4-oxo-3,4-dihydroquinazolin-3-yl)ethoxy]phenyl]-2-ethoxypropanoic acid phenyl glycinol salt;
(±)3-[4-[2-(2-Ethyl-4-oxo-3,4-dihydroquinazolin-3-yl)ethoxy]phenyl]-2-ethoxypropanoic acid methyl benzylamine salt;
(+)3-[4-[2-(2-Ethyl-4-oxo-3,4-dihydroquinazolin-3-yl)ethoxy]phenyl]-2-ethoxypropanoic acid methyl benzylamine salt;
(−)3-[4-[2-(2-Ethyl-4-oxo-3,4-dihydroquinazolin-3-yl)ethoxy]phenyl]-2-ethoxypropanoic acid methyl benzylamine salt;
(±)3-[4-[2-(2-Ethyl-4-oxo-3,4-dihydroquinazolin-3-yl)ethoxy]phenyl]-2-ethoxypropanoic acid dicyclohexylamine salt;
(+)3-[4-[2-(2-Ethyl-4-oxo-3,4-dihydroquinazolin-3-yl)ethoxy]phenyl]-2-ethoxypropanoic acid dicyclohexylamine salt;
(−)3-[4-[2-(2-Ethyl-4-oxo-3,4-dihydroquinazolin-3-yl)ethoxy]phenyl]-2-ethoxypropanoic acid dicyclohexylamine salt;
(±)3-[4-[2-(2-Ethyl-4-oxo-3,4-dihydroquinazolin-3-yl)ethoxy]phenyl]-2-ethoxypropanoic acid lysine salt;
(+)3-[4-[2-(2-Ethyl-4-oxo-3,4-dihydroquinazolin-3-yl)ethoxy]phenyl]-2-ethoxypropanoic acid lysine salt;
(−)3-[4-[2-(2-Ethyl-4-oxo-3,4-dihydroquinazolin-3-yl)ethoxy]phenyl]-2-ethoxypropanoic acid lysine salt;
(±)3-[4-[2-(2-Ethyl-4-oxo-3,4-dihydroquinazolin-3-yl)ethoxy]phenyl]-2-ethoxypropanoic acid tris (hydroxymethyl)amino methane salt;
(+)3-[4-[2-(2-Ethyl-4-oxo-3,4-dihydroquinazolin-3-yl)ethoxy]phenyl]-2-ethoxypropanoic acid tris (hydroxymethyl)amino methane salt;
(−)3-[4-[2-(2-Ethyl-4-oxo-3,4-dihydroquinazolin-3-yl)ethoxy]phenyl]-2-ethoxypropanoic acid tris (hydroxymethyl)amino methane salt;
(±)3-[4-[2-(2-Ethyl-4-oxo-3,4-dihydroquinazolin-3-yl)ethoxy]phenyl]-2-ethoxypropanoic acid N-octyl glucamine salt;
(+)3-[4-[2-(2-Ethyl-4-oxo-3,4-dihydroquinazolin-3-yl)ethoxy]phenyl]-2-ethoxypropanoic acid N-octyl glucamine salt;
(−)3-[4-[2-(2-Ethyl-4-oxo-3,4-dihydroquinazolin-3-yl)ethoxy]phenyl]-2-ethoxypropanoic acid N-octyl glucamine salt;
(±)3-[4-[2-(2-Ethyl-4-oxo-3,4-dihydroquinazolin-3-yl)ethoxy]phenyl]-2-ethoxypropanoic acid N-methyl glucamine salt;
(+)3-[4-[2-(2-Ethyl-4-oxo-3,4-dihydroquinazolin-3-yl)ethoxy]phenyl]-2-ethoxypropanoic acid N-methyl glucamine salt;
(−)3-[4-[2-(2-Ethyl-4-oxo-3,4-dihydroquinazolin-3-yl)ethoxy]phenyl]-2-ethoxypropanoic acid N-methyl glucamine salt;
(±)3-[4-[2-(2-Ethyl-4-oxo-3,4-dihydroquinazolin-3-yl) ethoxy]phenyl]-2-ethoxypropanoic acid amino guanidine hydrogen carbonate salt;
(+)3-[4-[2-(2-Ethyl-4-oxo-3,4-dihydroquinazolin-3-yl)ethoxy]phenyl]-2-ethoxypropanoic acid amino guanidine hydrogen carbonate salt;
(−)3-[4-[2-(2-Ethyl-4-oxo-3,4-dihydroquinazolin-3-l)ethoxy]phenyl]-2-ethoxypropanoic acid amino guanidine hydrogen carbonate salt;
(±)3-[4-[2-(2-Ethyl-4-oxo-3,4-dihydroquinazolin-3-yl)ethoxy]phenyl]-2-ethoxypropanoic acid lithium salt;
(+)3-[4-[2-(2-Ethyl-4-oxo-3,4-dihydroquinazolin-3-yl)ethoxy]phenyl]-2-ethoxypropanoic acid lithium salt;
(−)3-[4-[2-(2-Ethyl-4-oxo-3,4-dihydroquinazolin-3-yl)ethoxy]phenyl]-2-ethoxypropanoic acid lithium salt;
(±)3-[4-[2-(2-Ethyl-4-oxo-3,4-dihydroquinazolin-3-yl)ethoxy]phenyl]-2-ethoxypropanoic acid arginine salt;
(+)3-[4-[2-(2-Ethyl-4-oxo-3,4-dihydroquinazolin-3-yl)ethoxy]phenyl]-2-ethoxypropanoic acid arginine salt;
(−)3-[4-[2-(2-Ethyl-4-oxo-3,4-dihydroquinazolin-3-yl)ethoxy]phenyl]-2-ethoxypropanoic acid arginine salt;
(±)3-[4-[2-(2-Ethyl-4-oxo-3,4-dihydroquinazolin-3-yl)ethoxy]phenyl]-2-ethoxypropanoic acid metformin salt;
(+)3-[4-[2-(2-Ethyl-4-oxo-3,4-dihydroquinazolin-3-yl)ethoxy]phenyl]-2-ethoxypropanoic acid metformin salt;
(−)3-[4-[2-(2-Ethyl-4-oxo-3,4-dihydroquinazolin-3-yl)ethoxy]phenyl]-2-ethoxypropanoic acid metformin salt;
(±)3-[4-[2-(2-Ethyl-4-oxo-3,4-dihydroquinazolin-3-yl)ethoxy]phenyl]-2-ethoxypropanoic acid imidazole salt;
(+)3-[4-[2-(2-Ethyl-4-oxo-3,4-dihydroquinazolin-3-yl) ethoxy]phenyl]-2-ethoxypropanoic acid imidazole salt;
(−)3-[4-[2-(2-Ethyl-4-oxo-3,4-dihydroquinazolin-3-yl)ethoxy]phenyl]-2-ethoxypropanoic acid imidazole salt;
(±)3-[4-[2-(2-Ethyl-4-oxo-3,4-dihydroquinazolin-3-yl)ethoxy]phenyl]-2-ethoxypropanoic acid magnesium salt;
(+)3-[4-[2-(2-Ethyl-4-oxo-3,4-dihydroquinazolin-3-yl)ethoxy]phenyl]-2-ethoxypropanoic acid magnesium salt;
(−)3-[4-[2-(2-Ethyl-4-oxo-3,4-dihydroquinazolin-3-yl)ethoxy]phenyl]-2-ethoxypropanoic acid magnesium salt;
(±)3-[4-[2-(2-Morpholinyl-4-oxo-3,4-dihydroquinazolin-3-yl)ethoxy]phenyl]-2-ethoxypropanoic acid magnesium salt;
(+)3-[4-[2-(2-Morpholinyl-4-oxo-3,4-dihydroquinazolin-3-yl)ethoxy]phenyl]-2-ethoxypropanoic acid magnesium salt;
(−)3-[4-[2-(2-Morpholinyl-4-oxo-3,4-dihydroquinazolin-3-yl)ethoxy]phenyl]-2-ethoxypropanoic acid magnesium salt;
(±)3-[4-[2-(2-Piperidinyl-4-oxo-3,4-dihydroquinazolin-3-yl)ethoxy]phenyl]-2-ethoxypropanoic acid magnesium salt;
(+)3-[4-[2-(2-Piperidinyl-4-oxo-3,4-dihydroquinazolin-3-yl)ethoxy]phenyl]-2-ethoxypropanoic acid magnesium salt;
(−)3-[4-[2-(2-Piperidinyl-4-oxo-3,4-dihydroquinazolin-3-yl)ethoxy]phenyl]-2-ethoxypropanoic acid magnesium salt;
(±)3-[4-[2-(2-Azido-4-oxo-3,4-dihydroquinazolin-3-yl)ethoxy]phenyl]-2-ethoxypropanoic acid magnesium salt;
(+)3-[4-[2-(2-Azido-4-oxo-3,4-dihydroquinazolin-3-yl)ethoxy]phenyl]-2-ethoxypropanoic acid magnesium salt;
(−)3-[4-[2-(2-Azido-4-oxo-3,4-dihydroquinazolin-3-yl)ethoxy]phenyl]-2-ethoxypropanoic acid magnesium salt;
(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid magnesium salt;
(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid magnesium salt;
(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid magnesium salt;
(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid magnesium salt;
(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid magnesium salt;
(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid magnesium salt;
(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid potassium salt;
(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid potassium salt;
(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid potassium salt;
(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid potassium salt;
(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid potassium salt;
(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid potassium salt;
(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid calcium salt;
(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid calcium salt;
(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid calcium salt;
(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid calcium salt;
(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid calcium salt;
(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid calcium salt;
(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid lithium salt;
(+)3-[4-[2-(-Methyl-5-ethyl-7-oxo-3-propyl-6,7-dihydro-1H-pyrazolo[4,3-d]pyrimidin-6-yl)ethoxy]phenyl]-2-ethoxypropionic acid lithium salt;
(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid lithium salt;
(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid lithium salt;
(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid lithium salt;
(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid lithium salt;
(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid sodium salt;
(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid sodium salt;
(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid sodium salt;
(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid sodium salt;
(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid sodium salt;
(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid sodium salt;
(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid arginine salt;
(+)3-[4-[2-(-Methyl-5-ethyl-7-oxo-3-propyl-6,7-dihydro-1H-pyrazolo[4,3-d]pyrimidin-6-yl)ethoxy]phenyl]-2-ethoxypropionic acid arginine salt;
(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid arginine salt;
(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid arginine salt;
(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid arginine salt;
(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid arginine salt;
(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid methyl benzylamine salt;
(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid methyl benzylamine salt;
(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid methyl benzylamine salt;
(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid methyl benzylamine salt;
(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid methyl benzylamine salt;
(−)3-[4-[2-(1,5-Dimethyl-7-oxo-3-propyl-6,7-dihydro-1H-[4,3-d]pyrimidin-6-yl)ethoxy]phenyl]-2-ethoxypropionic acid methyl benzylamine salt;
(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid S-(+)-phenylglycinol salt;
(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid S-(+)-phenylglycinol salt;
(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid S-(+)-phenylglycinol salt;
(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid S-(+)-phenylglycinol salt;
(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid S-(+)-phenylglycinol salt;
(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid S-(+)-phenylglycinol salt;
(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid aminoguanidine salt;
(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid aminoguanidine salt;
(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid aminoguanidine salt;
(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid aminoguanidine salt;
(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid aminoguanidine salt;
(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid aminoguanidine salt;
(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid tromethamine salt;
(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid tromethamine salt;
(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid tromethamine salt;
(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid tromethamine salt;
(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid tromethamine salt;
(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid tromethamine salt;
(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid dicyclohexylamine salt;
(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid dicyclohexylamine salt;
(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid dicyclohexylamine salt;
(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid dicyclohexylamine salt;
(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid dicyclohexylamine salt;
(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid dicyclohexylamine salt;
(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid N-octylglucamine salt;
(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid N-octylglucamine salt;
(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid N-octylglucamine salt;
(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid N-octylglucamine salt;
(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid N-octylglucamine salt;
(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid N-octylglucamine salt;
(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid N-methylglucamine salt;
(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid N-methylglucamine salt;
(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid N-methylglucamine salt;
(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid N-methylglucamine salt;
(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid N-methylglucamine salt;
(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid N-methylglucamine salt;
(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid metformin salt;
(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid metformin salt;
(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid metformin salt;
(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid metformin salt;
(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid metformin salt;
(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid metformin salt;
(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid lysine salt;
(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid lysine salt;
(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid lysine salt;
(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid lysine salt;
(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid lysine salt;
(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid lysine salt;
(±)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid t-butylamine salt;
(+)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid t-butylamine salt;
(−)3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid t-butylamine salt;
(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid t-butylamine salt;
(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid t-butylamine salt;
(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid t-butylamine salt;
(±)2-Ethoxy-3-[4-[2-[2-ethyl-6-oxo-4-phenyl-1,6-dihydropyrimidin-1-yl]ethoxy]phenyl]propanoic acid potassium salt;
(+)2-Ethoxy-3-[4-[2-[2-ethyl-6-oxo-4-phenyl-1,6-dihydropyrimidin-1-yl]ethoxy]phenyl]propanoic acid potassium salt;
(−)2-Ethoxy-3-[4-[2-[2-ethyl-6-oxo-4-phenyl-1,6-dihydropyrimidin-1-yl]ethoxy]phenyl]propanoic acid potassium salt;
(±)2-Ethoxy-3-[4-[2-[2-ethyl-6-oxo-4-phenyl-1,6-dihydropyrimidin-1-yl]ethoxy]phenyl]propanoic acid magnesium salt;
(+)2-Ethoxy-3-[4-[2-[2-ethyl-6-oxo-4-phenyl-1,6-dihydropyrimidin-1-yl]ethoxy]phenyl]propanoic acid magnesium salt;
(−)2-Ethoxy-3-[4-[2-[2-ethyl-6-oxo-4-phenyl-1,6-dihydropyrimidin-1-yl]ethoxy]phenyl]propanoic acid magnesium salt;
(±)2-Ethoxy-3-[4-[2-[2-ethyl-6-oxo-4-phenyl-1,6-dihydropyrimidin-1-yl]ethoxy]phenyl]propanoic acid phenyl glycinol salt;
(+)2-Ethoxy-3-[4-[2-[2-ethyl-6-oxo-4-phenyl-1,6-dihydropyrimidin-1-yl]ethoxy]phenyl]propanoic acid phenyl glycinol salt;
(−)2-Ethoxy-3-[4-[2-[2-ethyl-6-oxo-4-phenyl-1,6-dihydropyrimidin-1-yl]ethoxy]phenyl]propanoic acid phenyl glycinol salt;
(±)2-Ethoxy-3-[4-[2-[2-ethyl-6-oxo-4-phenyl-1,6-dihydropyrimidin-1-yl]ethoxy]phenyl]propanoic acid methyl benzylamine salt;
(+)2-Ethoxy-3-[4-[2-[2-ethyl-6-oxo-4-phenyl-1,6-dihydropyrimidin-1-yl]ethoxy]phenyl]propanoic acid methyl benzyl amine salt;
(−)2-Ethoxy-3-[4-[2-[2-ethyl-6-oxo-4-phenyl-1,6-dihydropyrimidin-1-yl]ethoxy]phenyl]propanoic acid methyl benzylamine salt;
(±)2-Ethoxy-3-[4-[2-[2-ethyl-6-oxo-4-phenyl-1,6-dihydropyrimidin-1-yl]ethoxy]phenyl]propanoic acid sodium salt;
(+)2-Ethoxy-3-[4-[2-[2-ethyl-6-oxo-4-phenyl-1,6-dihydropyrimidin-1-yl]ethoxy]phenyl]propanoic acid sodium salt;
(−)2-Ethoxy-3-[4-[2-[2-ethyl-6-oxo-4-phenyl-1,6-dihydropyrimidin-1-yl]ethoxy]phenyl]propanoic acid sodium salt;
(±)2-Ethoxy-3-[4-[2-[2-ethyl-6-oxo-4-phenyl-1,6-dihydropyrimidin-1-yl]ethoxy]phenyl]propanoic acid L-lysine salt;
(+)2-Ethoxy-3-[4-[2-[2-ethyl-6-oxo-4-phenyl-1,6-dihydropyrimidin-1-yl]ethoxy]phenyl]propanoic acid L-lysine salt;
(−)2-Ethoxy-3-[4-[2-[2-ethyl-6-oxo-4-phenyl-1,6-dihydropyrimidin-1-yl]ethoxy]phenyl]propanoic acid L-lysine salt;
(±)2-Ethoxy-3-[4-[2-[2-ethyl-6-oxo-4-phenyl-1,6-dihydropyrimidin-1-yl]ethoxy]phenyl]propanoic acid t-butylamine salt;
(+)2-Ethoxy-3-[4-[2-[2-ethyl-6-oxo-4-phenyl-1,6-dihydropyrimidin-1-yl]ethoxy]phenyl]propanoic acid t-butylamine salt;
(−)2-Ethoxy-3-[4-[2-[2-ethyl-6-oxo-4-phenyl-1,6-dihydropyrimidin-1-yl]ethoxy]phenyl]propanoic acid t-butylamine salt;
(±)2-Ethoxy-3-[4-[2-[2-ethyl-6-oxo-4-phenyl-1,6-dihydropyrimidin-1-yl]ethoxy]phenyl]propanoic acid N-methyl glucamine salt;
(+)2-Ethoxy-3-[4-[2-[2-ethyl-6-oxo-4-phenyl-1,6-dihydropyrimidin-1-yl]ethoxy]phenyl]propanoic acid N-methyl glucamine salt;
(−)2-Ethoxy-3-[4-[2-[2-ethyl-6-oxo-4-phenyl-1,6-dihydropyrimidin-1-yl]ethoxy]phenyl]propanoic acid N-methyl glucamine salt;
(±)2-Ethoxy-3-[4-[2-[2-ethyl-6-oxo-4-phenyl-1,6-dihydropyrimidin-1-yl]ethoxy]phenyl]propanoic acid N-octyl glucamine salt;
(+)2-Ethoxy-3-[4-[2-[2-ethyl-6-oxo-4-phenyl-1,6-dihydropyrimidin-1-yl]ethoxy]phenyl]propanoic acid N-octyl glucamine salt;
(−)2-Ethoxy-3-[4-[2-[2-ethyl-6-oxo-4-phenyl-1,6-dihydropyrimidin-1-yl]ethoxy]phenyl]propanoic acid N-octyl glucamine salt;
(±)2-Ethoxy-3-[4-[2-[2-ethyl-6-oxo-4-phenyl-1,6-dihydropyrimidin-1-yl]ethoxy]phenyl]propanoic acid tris (hydroxymethyl) amino methane salt;
(+)2-Ethoxy-3-[4-[2-[2-ethyl-6-oxo-4-phenyl-1,6-dihydropyrimidin-1-yl]ethoxy]phenyl]propanoic acid tris (hydroxymethyl) amino methane salt;
(−)2-Ethoxy-3-[4-[2-[2-ethyl-6-oxo-4-phenyl-1,6-dihydropyrimidin-1-yl]ethoxy]phenyl]propanoic acid tris (hydroxymethyl) amino methane salt;
(±)2-Ethoxy-3-[4-[2-[2-ethyl-6-oxo-4-phenyl-1,6-dihydropyrimidin-1-yl]ethoxy]phenyl]propanoic acid lithium salt;
(+)2-Ethoxy-3-[4-[2-[2-ethyl-6-oxo-4-phenyl-1,6-dihydropyrimidin-1-yl]ethoxy]phenyl]propanoic acid lithium salt;
(−)2-Ethoxy-3-[4-[2-[2-ethyl-6-oxo-4-phenyl-1,6-dihydropyrimidin-1-yl]ethoxy]phenyl]propanoic acid lithium salt;
(±)2-Ethoxy-3-[4-[2-[2-ethyl-6-oxo-4-phenyl-1,6-dihydropyrimidin-1-yl]ethoxy]phenyl]propanoic acid calcium salt;
(+)2-Ethoxy-3-[4-[2-[2-ethyl-6-oxo-4-phenyl-1,6-dihydropyrimidin-1-yl]ethoxy]phenyl]propanoic acid calcium salt;
(−)2-Ethoxy-3-[4-[2-[2-ethyl-6-oxo-4-phenyl-1,6-dihydropyrimidin-1-yl]ethoxy]phenyl]propanoic acid calcium salt;
(±)2-Ethoxy-3-[4-[2-[2-ethyl-6-oxo-4-phenyl-1,6-dihydropyrimidin-1-yl]ethoxy]phenyl]propanoic acid L-arginine salt;
(+)2-Ethoxy-3-[4-[2-[2-ethyl-6-oxo-4-phenyl-1,6-dihydropyrimidin-1-yl]ethoxy]phenyl]propanoic acid L-arginine salt;
(−)2-Ethoxy-3-[4-[2-[2-ethyl-6-oxo-4-phenyl-1,6-dihydropyrimidin-1-yl]ethoxy]phenyl]propanoic acid L-arginine salt;
(±)2-Ethoxy-3-[4-[2-[2-ethyl-6-oxo-4-phenyl-1,6-dihydropyrimidin-1-yl]ethoxy]phenyl]propanoic acid metformin salt;
(+)2-Ethoxy-3-[4-[2-[2-ethyl-6-oxo-4-phenyl-1,6-dihydropyrimidin-1-yl]ethoxy]phenyl]propanoic acid metformin salt;
(−)2-Ethoxy-3-[4-[2-[2-ethyl-6-oxo-4-phenyl-1,6-dihydropyrimidin-1-yl]ethoxy]phenyl]propanoic acid metformin salt;
(±)2-Ethoxy-3-[4-[2-[2-ethyl-6-oxo-4-phenyl-1,6-dihydropyrimidin-1-yl]ethoxy]phenyl]propanoic acid dicyclohexylamine salt;
(+)2-Ethoxy-3-[4-[2-[2-ethyl-6-oxo-4-phenyl-1,6-dihydropyrimidin-1-yl]ethoxy]phenyl]propanoic acid dicyclohexylamine salt;
(−)2-Ethoxy-3-[4-[2-[2-ethyl-6-oxo-4-phenyl-1,6-dihydropyrimidin-1-yl]ethoxy]phenyl]propanoic acid dicyclohexylamine salt;
(±)2-Ethoxy-3-[4-[2-[2-ethyl-6-oxo-4-phenyl-1,6-dihydropyrimidin-1-yl]ethoxy]phenyl]propanoic acid aminoguanidine salt;
(+)2-Ethoxy-3-[4-[2-[2-ethyl-6-oxo-4-phenyl-1,6-dihydropyrimidin-1-yl]ethoxy]phenyl]propanoic acid aminoguanidine salt;
(−)2-Ethoxy-3-[4-[2-[2-ethyl-6-oxo-4-phenyl-1,6-dihydropyrimidin-1-yl]ethoxy]phenyl]propanoic acid aminoguanidine salt;
(±)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid aminoguanidine hydrogen carbonate salt;
(+)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid aminoguanidine hydrogen carbonate salt;
(−)3-[4-[2-(1,5-Dimethyl-7-oxo-3-propyl-6,7-dihydro-1H-pyrazolo[4,3-d]pyrimidin-6-yl)ethoxy]phenyl]-2-ethoxypropionic acid aminoguanidine hydrogen carbonate salt;
(±)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid aminoguanidine hydrogen carbonate salt;
(+)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid aminoguanidine hydrogen carbonate salt; or
(−)-3-[4-[2-(1-Methyl-5-ethyl-7-oxo-3-propyl-6,7-dihydro-1H-pyrazolo[4,3-d]pyrimidin-6-yl)ethoxy]phenyl]-2-ethoxypropionic acid aminoguanidine hydrogen carbonate salt.

12. A pharmaceutical composition which comprises a compound of formula (I) 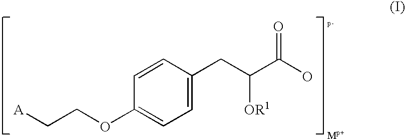
as defined in claim 1 and a pharmaceutically acceptable carrier, diluent, excipient or solvate.

13. A pharmaceutical composition which comprises a compound as claimed in claim 11 and a pharmaceutically acceptable carrier, diluent, excipient or solvate.

14. A composition which comprises a compound of formula (I) as defined in claim 1 and an HMG CoA reductase inhibitor, fibrate, nicotinic acid, cholestyramine, cholestipol, probucol or a mixture thereof and a pharmaceutically acceptable carrier, diluent, excipient or solvate.

15. A composition which comprises a compound as claimed in claim 11, and an HMG CoA reductase inhibitor, fibrate, nicotinic acid, cholestyramine, cholestipol, probucol or a mixture thereof and a pharmaceutically acceptable carrier, diluent, excipient or solvate.

16. A pharmaceutical composition as claimed in claim 12, in the form of a tablet, capsule, powder, syrup, solution or suspension.

17. A pharmaceutical composition as claimed in claim 13, in the form of a tablet, capsule, powder, syrup, solution or suspension.

18. A pharmaceutical composition as claimed in claim 14, in the form of a tablet, capsule, powder, syrup, solution or suspension.

19. A pharmaceutical composition as claimed in claim 15, in the form of a tablet, capsule, powder, syrup, solution or suspension.

20. A pharmaceutical composition as claimed in claim 12, for the treatment of type II diabetes, impaired glucose intolerance, leptin resistance, atherosclerosis, hyperlipidemia, disorders related to Syndrome X selected from hypertension, obesity, insulin resistance, coronary artery disease or other cardiovascular disorders; renal diseases selected from glomerulonephritis, glomerulosclerosis, nephrotic syndrome, hypertensive nephrosclerosis, or nephropathy; retinopathy, disorders related to endothelial cell activation, psoriasis, polycystic ovarian syndrome (PCOS), dementia, diabetic complications, eating disorders, osteoporosis, inflammatory bowel diseases, myotonic dystrophy, pancreatitis, retinopathy, arteriosclerosis, xanthoma or cancer.

21. A pharmaceutical composition as claimed in claim 13, for the treatment of type II diabetes, impaired glucose intolerance, leptin resistance, atherosclerosis, hyperlipidemia, disorders related to Syndrome X selected from hypertension, obesity, insulin resistance, coronary artery disease or other cardiovascular disorders; renal diseases selected from glomerulonephritis, glomerulosclerosis, nephrotic syndrome, hypertensive nephrosclerosis, or nephropathy; or retinopathy; disorders related to endothelial cell activation, psoriasis, polycystic ovarian syndrome (PCOS), dementia, diabetic complications, eating disorders, osteoporosis, inflammatory bowel diseases, myotonic dystrophy, pancreatitis, retinopathy, arteriosclerosis, xanthoma or cancer.

22. A pharmaceutical composition as claimed in claim 14, for the treatment of type II diabetes, impaired glucose intolerance, leptin resistance, atherosclerosis, hyperlipidemia, disorders related to Syndrome X selected from hypertension, obesity, insulin resistance, coronary artery disease or other cardiovascular disorders; renal diseases selected from glomerulonephritis, glomerulosclerosis, nephrotic syndrome, hypertensive nephrosclerosis, or nephropathy; retinopathy, disorders related to endothelial cell activation, psoriasis, polycystic ovarian syndrome (PCOS), dementia, diabetic complications, eating disorders, osteoporosis, inflammatory bowel diseases, myotonic dystrophy, pancreatitis, retinopathy, arteriosclerosis, xanthoma or cancer.

23. A pharmaceutical composition as claimed in claim 15, for the treatment of type II diabetes, impaired glucose intolerance, leptin resistance, atherosclerosis, hyperlipidemia, disorders related to Syndrome X selected from hypertension, obesity, insulin resistance, coronary artery disease or other cardiovascular disorders; renal diseases selected from glomerulonephritis, glomerulosclerosis, nephrotic syndrome, hypertensive nephrosclerosis, or nephropathy; retinopathy, disorders related to endothelial cell activation, psoriasis, polycystic ovarian syndrome (PCOS), dementia, diabetic complications, eating disorders, osteoporosis, inflammatory bowel diseases, myotonic dystrophy, pancreatitis, retinopathy, arteriosclerosis, xanthoma or cancer.

24. A method of treating hyperlipidemia, hypercholesteremia, hyperglycemia, osteoporosis, obesity, impaired glucose tolerance, atherosclerosis, leptin resistance, insulin resistance or diseases in which insulin resistance is the underlying pathophysiological mechanism comprising administering a compound of formula (I) as defined in claim 1 to a patient in need thereof.

25. A method of treating hyperlipidemia, hypercholesteremia, hyperglycemia, osteoporosis, obesity, impaired glucose tolerance, atherosclerosis, leptin resistance, insulin resistance or diseases in which insulin resistance is the underlying pathophysiological mechanism comprising administering a compound as defined in claim 11 to a patient in need thereof.

26. A method of treating hyperlipidemia, hypercholesteremia, hyperglycemia, osteoporosis, obesity, impaired glucose tolerance, atherosclerosis, leptin resistance, insulin resistance or diseases in which insulin resistance is the underlying pathophysiological mechanism comprising administering a pharmaceutical composition as defined in claim 12 to a patient in need thereof.

27. A method of treating hyperlipidemia, hypercholesteremia, hyperglycemia, osteoporosis, obesity, impaired glucose tolerance, atherosclerosis, leptin resistance, insulin resistance or diseases in which insulin resistance is the underlying pathophysiological mechanism comprising administering a pharmaceutical composition as defined in claim 13 to a patient in need thereof.

28. A method of treating hyperlipidemia, hypercholesteremia, hyperglycemia, osteoporosis, obesity, impaired glucose tolerance, atherosclerosis, leptin resistance, insulin resistance or diseases in which insulin resistance is the underlying pathophysiological mechanism comprising administering a composition as defined in claim 14 to a patient in need thereof.

29. A method of treating hyperlipidemia, hypercholesteremia, hyperglycemia, osteoporosis, obesity, impaired glucose tolerance, atherosclerosis, leptin resistance, insulin resistance or diseases in which insulin resistance is the underlying pathophysiological mechanism comprising administering a composition as defined in claim 15 to a patient in need thereof.

30. A method according to claim 24, wherein the disease is type II diabetes, impaired glucose tolerance, disorders related to Syndrome X selected from hypertension, obesity, insulin resistance, atherosclerosis, coronary artery disease or other cardiovascular disorders; renal diseases selected from glomerulonephritis, glomerulosclerosis, nephrotic syndrome, hypertensive nephrosclerosis, or nephropathy; retinopathy, disorders to related endothelial cell activation, psoriasis, polycystic ovarian syndrome (PCOS), dementia, diabetic complications, inflammatory bowel diseases, myotonic dystrophy, pancreatitis, arteriosclerosis, xanthoma, eating disorders, cancer, osteoporosis or inflammation.

31. A method according to claim 25, wherein the disease is type II diabetes, impaired glucose tolerance, disorders related to Syndrome X selected from hypertension, obesity, insulin resistance, atherosclerosis, coronary artery disease or other cardiovascular disorders; renal diseases selected from glomerulonephritis, glomerulosclerosis, nephrotic syndrome, hypertensive nephrosclerosis, or nephropathy; retinopathy, disorders to related endothelial cell activation, psoriasis, polycystic ovarian syndrome (PCOS), dementia, diabetic complications, inflammatory bowel diseases, myotonic dystrophy, pancreatitis, arteriosclerosis, xanthoma, eating disorders, cancer, osteoporosis or inflammation.

32. A method according to claim 26, wherein the disease is type II diabetes, impaired glucose tolerance, disorders related to Syndrome X selected from hypertension, obesity, insulin resistance, atherosclerosis, coronary artery disease or other cardiovascular disorders; renal diseases selected from glomerulonephritis, glomerulosclerosis, nephrotic syndrome, hypertensive nephrosclerosis, or nephropathy; retinopathy, disorders to related endothelial cell activation, psoriasis, polycystic ovarian syndrome (PCOS), dementia, diabetic complications, inflammatory bowel diseases, myotonic dystrophy, pancreatitis, arteriosclerosis, xanthoma, eating disorders, cancer, osteoporosis or inflammation.

33. A method according to claim 27, wherein the disease is type II diabetes, impaired glucose tolerance, disorders related to Syndrome X selected from hypertension, obesity, insulin resistance, atherosclerosis, coronary artery disease or other cardiovascular disorders; renal diseases selected from glomerulonephritis, glomerulosclerosis, nephrotic syndrome, hypertensive nephrosclerosis, or nephropathy; retinopathy, disorders to related endothelial cell activation, psoriasis, polycystic ovarian syndrome (PCOS), dementia, diabetic complications, inflammatory bowel diseases, myotonic dystrophy, pancreatitis, arteriosclerosis, xanthoma, eating disorders, cancer, osteoporosis or inflammation.

34. A method according to claim 28, wherein the disease is type II diabetes, impaired glucose tolerance, disorders related to Syndrome X selected from hypertension, obesity, insulin resistance, atherosclerosis, coronary artery disease or other cardiovascular disorders; renal diseases selected from glomerulonephritis, glomerulosclerosis, nephrotic syndrome, hypertensive nephrosclerosis, or nephropathy; retinopathy, disorders to related endothelial cell activation, psoriasis, polycystic ovarian syndrome (PCOS), dementia, diabetic complications, inflammatory bowel diseases, myotonic dystrophy, pancreatitis, arteriosclerosis, xanthoma, eating disorders, cancer,osteoporosis or inflammation.

35. A method according to claim 29, wherein the disease is type II diabetes, impaired glucose tolerance, disorders related to Syndrome X selected from hypertension, obesity, insulin resistance, atherosclerosis, coronary artery disease or other cardiovascular disorders; renal diseases selected from glomerulonephritis, glomerulosclerosis, nephrotic syndrome, hypertensive nephrosclerosis, or nephropathy; retinopathy, disorders to related endothelial cell activation, psoriasis, polycystic ovarian syndrome (PCOS), dementia, diabetic complications, inflammatory bowel diseases, myotonic dystrophy, pancreatitis, arteriosclerosis, xanthoma, eating disorders, cancer, osteoporosis or inflammation.

36. A method for the treatment of disorders related to Syndrome X, which comprises administering an agonist of PPARα and/or PPARγ of formula (I) as claimed in claim 1 to a patient in need thereof.

37. A method for the treatment of disorders related to Syndrome X, which comprises administering an agonist of PPARα and/or PPARγ as claimed in claim 11 to a patient in need thereof.

38. A method for the treatment of disorders related to Syndrome X, which comprises administering a pharmaceutical composition according to claim 12 comprising an agonist of PPARα and/or PPARγ to a patient in need thereof.

39. A method for the treatment of disorders related to Syndrome X, which comprises administering a pharmaceutical composition according to claim 13 comprising an agonist of PPARα and/or PPARγ to a patient in need thereof

40. A method for the treatment of disorders related to Syndrome X, which comprises administering a pharmaceutical composition according to claim 14 comprising an agonist of PPARα and/or PPARγ to a patient in need thereof.

41. A method for the treatment of disorders related to Syndrome X, which comprises administering a pharmaceutical composition according to claim 15 comprising an agonist of PPARα and/or PPARγ to a patient in need thereof.

42. A method of reducing total cholesterol, body weight, blood plasma glucose, triglycerides, LDL, VLDL or free fatty acids or increasing HDL in the plasma comprising administering a compound of formula (I), as defined in claim 1 to a patient in need thereof.

43. A method of reducing total cholesterol, body weight, blood plasma glucose, triglycerides, LDL, VLDL or free fatty acids or increasing HDL in the plasma comprising administering a compound as claimed in claim 11 to a patient in need thereof.

44. A method of reducing total cholesterol, body weight, blood plasma glucose, triglycerides, LDL, VLDL or free fatty acids or increasing HDL in the plasma comprising administering a pharmaceutical composition according to claim 12 to a patient in need thereof.

45. A method of reducing total cholesterol, body weight, blood plasma glucose, triglycerides, LDL, VLDL or free fatty acids or increasing HDL in the plasma comprising administering a pharmaceutical composition according to claim 13 to a patient in need thereof.

46. A method of reducing total cholesterol, body weight, blood plasma glucose, triglycerides, LDL, VLDL or free fatty acids or increasing HDL in the plasma comprising administering a pharmaceutical composition according to claim 14 to a patient in need thereof.

47. A method of reducing total cholesterol, body weight, blood plasma glucose, triglycerides, LDL, VLDL or free fatty acids or increasing HDL in the plasma comprising administering a pharmaceutical composition according to claim 15 to a patient in need thereof.

48. A method of treating hyperlipemia, hypercholesteremia, hyperglycemia, osteoporosis, obesity, impaired glucose tolerance, atherosclerosis, leptin resistance, insulin resistance, or diseases in which insulin resistance is the underlying pathophysiological mechanism comprising administering to a patient in need thereof an effective amount of a compound of formula (1) as defined in claim 1 in combination/concomittant with a HMG CoA reductase inhibitor, fibrate, nicotinic acid, cholestyramine, colestipol or probucol or a mixture thereof within such a period so as to act synergistically.

49. A method of treating hyperlipemia, hypercholesteremia, hyperglycemia, osteoporosis, obesity, impaired glucose tolerance, atherosclerosis, leptin resistance, insulin resistance, or diseases in which insulin resistance is the underlying pathophysiological mechanism comprising administering to a patient in need thereof an effective amount of a compound as claimed in claim 11 in combination/concomittant with a HMG CoA reductase inhibitor, fibrate, nicotinic acid, cholestyramine, colestipol or probucol or a mixture thereof within such a period so as to act synergistically.

50. A method of treating hyperlipemia, hypercholesteremia, hyperglycemia, osteoporosis, obesity, impaired glucose tolerance, atherosclerosis, leptin resistance, insulin resistance, or diseases in which insulin resistance is the underlying pathophysiological mechanism comprising administering to a patient in need thereof an effective amount of a pharmaceutical composition according to claim 12 in combination/concomittant with a HMG CoA reductase inhibitor, fibrate, nicotinic acid, cholestyramine, colestipol or probucol or a mixture thereof within such a period so as to act synergistically.

51. A method of treating hyperlipemia, hypercholesteremia, hyperglycemia, osteoporosis, obesity, impaired glucose tolerance, atherosclerosis, leptin resistance, insulin resistance, or diseases in which insulin resistance is the underlying pathophysiological mechanism comprising administering to a patient in need thereof an effective amount of a pharmaceutical composition according to claim 13 in combination/concomittant with a HMG CoA reductase inhibitor, fibrate, nicotinic acid, cholestyramine, colestipol or probucol or a mixture thereof within such a period so as to act synergistically.

52. A method according to claim 48, wherein the disease is type II diabetes, impaired glucose tolerance, disorders related to Syndrome X selected from hypertension, obesity, insulin resistance, atherosclerosis, coronary artery disease or other cardiovascular disorders; renal diseases selected from glomerulonephritis, glomerulosclerosis, nephrotic syndrome, hypertensive nephrosclerosis, or nephropathy; retinopathy, disorders to related endothelial cell activation, psoriasis, polycystic ovarian syndrome (PCOS), dementia, diabetic complications, inflammatory bowel diseases, myotonic dystrophy, pancreatitis, arteriosclerosis, xanthoma, eating disorders, cancer, osteoporosis or inflammation.

53. A method according to claim 49, wherein the disease is type II diabetes, impaired glucose tolerance, disorders related to Syndrome X selected from hypertension, obesity, insulin resistance, atherosclerosis, coronary artery disease or other cardiovascular disorders; renal diseases selected from glomerulonephritis, glomerulosclerosis, nephrotic syndrome, hypertensive nephrosclerosis, or nephropathy; retinopathy, disorders to related endothelial cell activation, psoriasis, polycystic ovarian syndrome (PCOS), dementia, diabetic complications, inflammatory bowel diseases, myotonic dystrophy, pancreatitis, arteriosclerosis, xanthoma, eating disorders, cancer, osteoporosis or inflammation.

54. A method according to claim 50, wherein the disease is type II diabetes, impaired glucose tolerance, disorders related to Syndrome X selected from hypertension, obesity, insulin resistance, atherosclerosis, coronary artery disease or other cardiovascular disorders; renal diseases selected from glomerulonephritis, glomerulosclerosis, nephrotic syndrome, hypertensive nephrosclerosis, or nephropathy; retinopathy, disorders to related endothelial cell activation, psoriasis, polycystic ovarian syndrome (PCOS), dementia, diabetic complications, inflammatory bowel diseases, myotonic dystrophy, pancreatitis, arteriosclerosis, xanthoma, eating disorders, cancer, osteoporosis or inflammation.

55. A method according to claim 51, wherein the disease is type II diabetes, impaired glucose tolerance, disorders related to Syndrome X selected from hypertension, obesity, insulin resistance, atherosclerosis, coronary artery disease or other cardiovascular disorders; renal diseases selected from glomerulonephritis, glomerulosclerosis, nephrotic syndrome, hypertensive nephrosclerosis, or nephropathy; retinopathy, disorders to related endothelial cell activation, psoriasis, polycystic ovarian syndrome (PCOS), dementia, diabetic complications, inflammatory bowel diseases, myotonic dystrophy, pancreatitis, arteriosclerosis, xanthoma, eating disorders, cancer, osteoporosis or inflammation.

56. A method for the treatment of disorders related to Syndrome X, which comprises administering to a patient in need thereof an agonist of PPARα and/or PPARγ of formula (I) as claimed in claim 1 in combination/concomittant with a HMG CoA reductase inhibitor, fibrate, nicotinic acid, cholestyramine, colestipol or probucol or a mixture thereof within such a period as to act synergistically.

57. A method for the treatment of disorders related to Syndrome X, which comprises administering to a patient in need thereof an agonist of PPARα and/or PPARγ as claimed in claim 11 in combination/concomittant with a HMG CoA reductase inhibitor, fibrate, nicotinic acid, cholestyramine, colestipol or probucol or a mixture thereof within such a period as to act synergistically.

58. A method for the treatment of disorders related to Syndrome X, which comprises administering to a patient in need thereof a pharmaceutical composition according to claim 12, comprising an agonist of PPARα and/or PPARγ in combination/concomittant with a HMG CoA reductase inhibitor, fibrate, nicotinic acid, cholestyramine, colestipol or probucol or a mixture thereof within such a period as to act synergistically.

59. A method for the treatment of disorders related to Syndrome X, which comprises administering to a patient in need thereof a composition according to claim 13, comprising an agonist of PPARα and/or PPARγ and a HMG CoA reductase inhibitor, fibrate, nicotinic acid, cholestyramine, colestipol or probucol or a mixture thereof within such a period as to act synergistically.

60. A method of reducing plasma glucose, triglycerides, total cholesterol, LDL, VLDL or free fatty acids or increasing HDL in the plasma, which comprises administering a compound of formula (I) claimed in claim 1, in combination/concomittant with a HMG CoA reductase inhibitor, fibrate, nicotinic acid, cholestyramine, colestipol or probucol or a mixture thereof which may be administered together or within such a period as to act synergistically together to a patient in need thereof.

61. A method of reducing plasma glucose, triglycerides, total cholesterol, LDL, VLDL or free fatty acids or increasing HDL in the plasma, which comprises administering a compound as claimed in claim 11, in combination/concomittant with a HMG CoA reductase inhibitor, fibrate, nicotinic acid, cholestyramine, colestipol or probucol or a mixture thereof which may be administered together or within such a period as to act synergistically together to a patient in need thereof.

62. A method of reducing plasma glucose, triglycerides, total cholesterol, LDL, VLDL or free fatty acids or increasing HDL in the plasma, which comprises administering a pharmaceutical composition according to claim 12, in combination/concomittant with a HMG CoA reductase inhibitor, fibrate, nicotinic acid, cholestyramine, colestipol or probucol or a mixture thereof which may be administered together or within such a period as to act synergistically together to a patient in need thereof.

63. A method of reducing plasma glucose, triglycerides, total cholesterol, LDL, VLDL or free fatty acids or increasing HDL in the plasma, which comprises administering a pharmaceutical composition according to claim 13, in combination/concomittant with a HMG CoA reductase inhibitor, fibrate, nicotinic acid, cholestyramine, colestipol or probucol or a mixture thereof which may be administered together or within such a period as to act synergistically together to a patient in need thereof.

64. The process as claimed in claim 5, wherein the alcohol is selected from a group consisting of ethanol, methanol, isopropanol and butanol; ketone is selected from a group consisting of acetone, diethyl ketone, and methyl ethyl ketone; and ether is selected from a group consisting of diethyl ether, ether, tetrahydrofuran, dioxane, and dibutyl ether.

65. The process as claimed in claim 6, wherein the alcohol is selected from a group consisting of ethanol, methanol, isopropanol and butanol; ketone is selected from a group consisting of acetone, diethyl ketone, and methyl ethyl ketone; and ether is selected from a group consisting of diethyl ether, ether, tetrahydrofuran, dioxane, and dibutyl ether.
